(19) United States
(12) Patent Application Publication
(10) Pub. No.: US 2005/0002917 A1
(43) Pub. Date: Jan. 6, 2005

(54) Regulation of Acheron expression

(76) Inventors: Lawrence M. Schwartz, Pelham, MA (US); Zhaohui Wang, Newton, MA (US); Christos Valavanis, Athens (GR)

Correspondence Address: FISH & RICHARDSON PC, 225 FRANKLIN ST, BOSTON, MA, 02110, US

(21) Appl. No.: 10/841,798

(22) Filed: May 7, 2004

Related U.S. Application Data
(60) Provisional application No. 60/468,708, filed on May 7, 2003.

(51) Int. Cl. G01N 33/53; G01N 33/567; C12P 21/04; A61K 48/00

(52) U.S. Cl. 424/93.21; 435/455; 435/366; 435/7.2

(57) ABSTRACT

The invention relates to novel apoptosis-associated nucleic acids and polypeptides and methods for use thereof, including methods of treatment of disorders associated with aberrant cellular proliferation, differentiation, or degeneration. Included are methods of enhancing the success of cell transplantation and cell-based genetic therapy procedures.

### CLAIM OF PRIORITY

This application claims priority under 35 USC § 119(e) to U.S. Patent Application Ser. No. 60/468,708, filed on May 7, 2003, the entire contents of which are incorporated herein by reference.

### FEDERALLY SPONSORED RESEARCH OR DEVELOPMENT

This invention was made in part with Government support under Grant No. GM40458 awarded by the National Institutes of Health. The Government has certain rights in the invention.

### TECHNICAL FIELD

This invention relates to regulation of Acheron expression.

### BACKGROUND

A general trend in vertebrate organogenesis is that many more cells are produced than will ultimately be required. Cell-cell interactions allow cells to determine if they are valuable members of the developmental community or surplus individuals that are not needed for tissue formation. This latter population fails to activate the appropriate survival programs and instead undergoes apoptosis. This game of cellular musical chairs serves to remove potentially deleterious mitotically-competent cells that pose a risk of transformation, e.g., cancerous or pre-cancerous cells. While the molecular machinery that mediates the execution phase of apoptosis has been studied, much less is know about the signal transduction pathways that activate this program in a lineage-specific manner.

### SUMMARY

The present invention is based, in part, on the discovery of a novel death-associated gene, initially cloned from the tobacco hawk moth Manduca sexta, termed Acheron, after the name of the river that leads to the realm of the dead in ancient Greek mythology.

In one aspect, the invention provides isolated engineered cells having an altered level of Acheron activity, e.g., reduced or increased Acheron activity. The cells can be any type of cell, including myoblasts, neural stem cells, and hematopoietic stem cells. In some embodiments, the cells include an exogenous gene. The cells can have permanently or transiently altered, e.g., reduced or increased Acheron activity, e.g., cells expressing or treated with an Acheron inhibitor. The invention additionally provides methods for preparing such cells.

As used herein, an Acheron inhibitor reduces Acheron expression or activity. Exemplary Acheron inhibitors include an Acheron-specific antibody, an antisense nucleic acid complementary to an Acheron nucleic acid, a small inhibitory RNA that cleaves an Acheron mRNA, a ribozyme that cleaves an Acheron nucleic acid, and a dominant negative Acheron polypeptide. In some embodiments, the Acheron inhibitor is a CASK-C dominant negative. In some embodiments, the invention includes compositions including one or more inhibitors of Acheron activity, and a pharmaceutically acceptable carrier.

In another aspect, the invention provides methods for preparing a cells for implantation into a recipient. The method includes contacting the cell with an Acheron inhibitor in an amount effective to reduce Acheron expression or activity within the cell.

The invention further provides kits comprising an Acheron inhibitor, and instructions for use in a method of preparing cells for transplantation.

The invention also provides methods for identifying candidate compounds for the treatment of disorders associated with aberrant apoptosis or cellular differentiation, e.g., as described herein. The method includes providing an Acheron nucleic acid molecule or polypeptide; contacting the Acheron nucleic acid molecule or polypeptide with a test compound under conditions in which the nucleic acids expression or polypeptide activity can be determined; and evaluating any effect of the test compound on the expression of the Acheron nucleic acid or an activity of the Acheron polypeptide. A test compound that modulates the expression of the Acheron nucleic acid or an activity of the Acheron polypeptide is a candidate compound for the treatment of a disorder associated with apoptosis or cellular differentiation. In some embodiments, the Acheron nucleic acid molecule or polypeptide is in a cell.

In some embodiments, the method also includes selecting a candidate compound that increases expression of the Acheron nucleic acid or the activity of the Acheron polypeptide; and evaluating the candidate compound in a mammal having a disorder associated with aberrant cellular proliferation.

In some embodiments, the method also includes selecting a compound that decreases the expression of the Acheron nucleic acid or the activity of the Acheron polypeptide; and evaluating the compound in a mammal having a disorder associated with aberrant cellular degeneration, e.g., muscular dystrophy.

In some embodiments, the mammal is a human subject in a clinical trial.

In another aspect, the invention provides isolated nucleic acid molecules including:
(a) isolated nucleic acid molecules encode Acheron polypeptides of 5 to 490 contiguous amino acids within SEQ ID NO:4, wherein the polypeptides have a measurable affect on apoptosis or cellular differentiation that is at least 25% of the measured affect of the full-length Acheron polypeptide, and
(b) isolated nucleic acid molecules that encode dominant negative Acheron polypeptides of 5 to 457 contiguous amino acids within amino acid locations 34-491 of SEQ ID NO:4.

The invention also includes vectors including the nucleic acid molecules described herein, and, in some cases, also including a nucleic acid sequence encoding a heterologous polypeptide, and host cells that contain the nucleic acid molecules described herein, e.g., mammalian host cells, e.g., human or non-human mammalian host cells.

The invention also includes isolated polypeptides including:
(a) an Acheron polypeptide comprising a sequence of 5 to 490 contiguous amino acids within SEQ ID NO:4, wherein the polypeptide has a measurable affect on apoptosis or cellular differentiation that is at least 25% of the measured affect of the full-length Acheron polypeptide; and
(b) a dominant negative Acheron polypeptide comprising a sequence of 5 to 457 contiguous amino acids within amino acid locations 34-491 of SEQ ID NO:4.

In some embodiments, the polypeptides also include a heterologous amino acid sequence, e.g., dystrophin. In some embodiments, the polypeptide is an active fragment of the amino acid sequence of SEQ ID NO:4 that retains at least one biological activity of the full length protein, e.g., regulation of apoptosis or differentiation, or binding of parkin, calcium/calmodulin-dependent serine protein kinase C (CASK-C) and/or Ariadne. In some embodiments, the polypeptide is a fragment of the amino acid sequence of SEQ ID NO:4 that acts as a dominant negative, e.g., a fragment lacking the first 33 amino acids but including amino acids 34-491 of SEQ ID NO:4. For example, the polypeptides can be naturally occurring allelic variants of a polypeptide including the amino acid sequence of SEQ ID NO:4, wherein the polypeptide is encoded by a nucleic acid that hybridizes to a nucleic acid molecule including SEQ ID NO:3 or 5, or a complement thereof, under stringent conditions. The invention also includes methods for producing the new polypeptides described herein, e.g., by culturing the host cells described herein under conditions in which the nucleic acid molecule encoding the polypeptide is expressed.

In addition, the invention provides compositions including a nucleic acid or polypeptide described herein. In some embodiments, the compositions also include a physiologically acceptable carrier.

In another aspect, the invention includes isolated antibodies, or antigen-binding portions thereof (e.g., Fv, Fab, or F(ab′)₂) that bind to an Acheron polypeptide. The isolated antibody can be, for example, a monoclonal, polyclonal, or monospecific antibody.

In another aspect, the invention includes methods of treating a subject in need of a cellular implant. The methods include administering to the subject an effective amount of cells having reduced Acheron activity.

The invention further provides methods of treating a subject having a disorder associated with abnormal cellular degeneration. The methods include administering to the subject cells comprising an amount of an Acheron inhibitor effective to reduce Acheron activity in the cells compared to wild type cells. The Acheron inhibitor can be, for example, an Acheron-specific antibody, an antisense nucleic acid complementary to an Acheron nucleic acid, a small inhibitory RNA that cleaves an Acheron mRNA, a ribozyme that cleaves an Acheron nucleic acid, a nucleic acid molecular that encodes a dominant negative Acheron polypeptide, and a dominant negative Acheron polypeptide.

In another aspect, the invention features methods of treating a subject who has a disease characterized by abnormal cellular degeneration, as described herein. The methods include administering an inhibitor of Acheron activity to the subject. In some embodiments, the inhibitor of Acheron activity can include one or more of an antisense nucleic acid, a small interfering nucleic acid, a ribozyme, a dominant negative polypeptide, a kinase inhibitor, or a nucleic acid encoding a dominant negative, e.g., an Acheron dominant negative or a CASK-C dominant negative.

The invention additionally features methods of treating a subject having a disease characterized by aberrant cellular proliferation or differentiation, e.g., as described herein. The methods include administering one or more enhancers of Acheron activity. In some embodiments, the enhancer of Acheron activity includes a nucleic acid molecule or polypeptide described herein.

In another aspect, the invention provides methods for detecting the presence of an Acheron polypeptide as described herein in a sample. The methods include contacting the sample with a compound that selectively binds to the polypeptide; and determining whether the compound binds to the polypeptide in the sample. In some embodiments, the compound that binds to the polypeptide is an antibody. In some embodiments, the polypeptide is Acheron, CASK-C, or Ariadne.

The invention also provides kits including one or more compounds that selectively bind to an Acheron polypeptide or nucleic acid molecule as described herein, and instructions for use.

A method for detecting the presence of an Acheron nucleic acid molecule in a sample. The method includes contacting the sample with a nucleic acid probe or primer that selectively hybridizes to the nucleic acid molecule, and determining whether the nucleic acid probe or primer binds to a nucleic acid molecule in the sample. In some embodiments, the sample comprises mRNA molecules and is contacted with a nucleic acid probe. The invention also includes a kit comprising a compound that selectively hybridizes to a nucleic acid molecule of claim 1 and instructions for use.

The invention additionally provides methods for identifying compounds that bind to a polypeptide described herein, e.g., Acheron. The methods include contacting the polypeptide or a cell expressing the polypeptide, with a test compound; and determining whether the polypeptide binds to the test compound. In some embodiments, the binding of the test compound to the polypeptide is detected by a method selected from the group consisting of detection of binding by directly detecting test compound/polypeptide binding; detection of binding using a competition binding assay; detection of binding using by detecting subcellular localization of Acheron; and detection of binding using an assay for Acheron-mediated apoptosis.

In another aspect, the invention provides methods for modulating the activity of a polypeptide described herein, e.g., Acheron. The method includes contacting the polypeptide, or a cell expressing the polypeptide, with a compound that binds to the polypeptide in a sufficient concentration to modulate the activity of the polypeptide.

The invention also provides methods for identifying compounds that modulate the expression or activity of a polypeptide or nucleic acid described herein. The method includes contacting the polypeptide or nucleic acid with a test compound; and determining an effect of the test compound on the expression or activity of the polypeptide or nucleic acid, to thereby identify a compound that modulates the expression or activity of the polypeptide or nucleic acid.

In another aspect, the invention includes transgenic animals, e.g., animals at least some of whose somatic and germ cells comprise at least one Acheron transgene as described herein.

Also within the invention is the use of Acheron and/or any of the inhibitors of Acheron activity described herein, e.g., an antisense nucleic acid, a small interfering nucleic acid, a ribozyme, an antibody, a dominant negative polypeptide, a kinase inhibitor, or a nucleic acid encoding a dominant negative, in the manufacture of a medicament for the treatment or prevention of disorders associated with aberrant cellular degeneration. The medicament can be used in a method for treating or preventing disorders associated with aberrant cellular degeneration in a patient suffering from or at risk for a disorder associated with aberrant cellular degeneration.

Further, within the invention is the use of Acheron and/or any of the enhancers of Acheron activity described herein, e.g., Acheron nucleic acids or polypeptides or active fragments thereof, in the manufacture of a medicament for the treatment or prevention of disorders associated with aberrant cellular differentiation and/or proliferation. The medicament can be used in a method for treating or preventing disorders associated with aberrant cellular differentiation and/or proliferation in a patient suffering from or at risk for a disorder associated with aberrant cellular differentiation and/or proliferation.

Also within the invention is an Acheron nucleic acid, polypeptide, antibody, antisense nucleic acid, a small interfering nucleic acid, a ribozyme, a dominant negative polypeptide, or a nucleic acid encoding a dominant negative for use in treating disorders associated with aberrant cellular degeneration, differentiation and/or proliferation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is a reproduction of a Northern blot of Manduca sexta intersegrnental muscle (ISM) RNA hybridized with Acheron cDNA. Treatment of day 17 animals with 25 μg of the steroid 20-hydroxyecdysone (20-HE) delays ISM death. d=day of pupal-adult development; hrs=hours after adult emergence.

FIG. 1B is a reproduction of the same Northern blot of Manduca sexta intersegmental muscle (ISM) RNA shown in FIG. 1A, stripped and reprobed with the constitutively expressed ubiquitin-fusion 80 cDNA 23 gene as a loading control.

FIG. 1C is a reproduction of a Northern blot of day 18 moth tissues probed with Acheron cDNA. ISM=intersegmental muscle; FM=flight muscle; FB=fat body; MT=Malpighian tubule; MAC=male accessory gland; OV=ovary.

FIG. 2 is a bar graph showing levels of cell death in control and Acheron over-expressing cells as determined by trypan blue assays cultured in growth medium (GM), or cultured for 1 day or 2 days in differentiation medium.

FIGS. 3A-3C are a series of reproductions of Western blots demonstrating temporal expression patterns of MyoD (top row) and Myf5 (bottom row) proteins in C2C12 cells transfected with empty vector (3A), Acheron (3B) or tAcheron (3C) constructs. Protein samples were collected from cells cultured in GM (G), or 1 day, 2 days or 3 days in DM. 20 μg of proteins from each sample were analyzed for Western blots. Lane A in FIG. 3B was a sample collected from Acheron over-expressing cells after 3 days in DM and floating apoptotic cells were removed with PBS wash.

FIG. 3D is a reproduction of a Western blot showing that ectopic MyoD can be expressed in C2C12 myoblasts expressing truncated Acheron. MHC staining in the accompanying micrographs demonstrates that these cells differentiate into myotubes.

FIGS. 3E and 3F are a pair of photomicrographs of ICC staining of MHC in tAch cells (3E) and tAch-MyoD co-expressing cells (3F) after 3 days in DM. Forced expression of MyoD (F) reinstates the differentiation inhibited by tAch (E).

FIGS. 4A-4D are a series of reproductions of Western blots analyzing the expression of Bcl-2 (top row) and Bax (middle row) proteins in C₂C₁₂ cells transfected with empty vector (4A), Acheron (4B), truncated Acheron (tAch; 4C) or antisense Acheron (4D). Total proteins were collected in GM (G), and in DM for 1, 2, or 3 days. The bottom row shows the same blots reprobed with M56, a subunit of 26S proteosome, as an internal control for protein loading.

FIG. 4E is a bar graph illustrating the results of quantitative analysis of the ratio of Bcl-2/Bax expression.

FIG. 5 is a schematic illustration of a model of the effects of Acheron mis-expression on C₂C₁₂ cells. Under differentiation conditions, Acheron over-expression reduces Myf5 expression, suppresses up-regulation of Bcl-2 and causes apoptosis, although it allows the cells to undergo differentiation to form myotubes. In contrast, the dominant negative Acheron, tAch, results in greatly increased ‘reserve’ cell population and decreased differentiation.

FIG. 6 is a schematic illustration of the putative Acheron protein structure. LA: Lupus antigen; RBD: RNA binding domain; NLS: nuclear localization signal. Acheron proteins are structurally related to La proteins, but define a novel subfamily.

FIG. 7 is a bar graph showing relative Acheron mRNA levels in human fetal and adult tissues and representative tumor cell lines. The histogram was obtained by phosphorimager densitometric analysis of normalized mRNA dot blots.

FIG. 8 is a sequence listing showing the cDNA (SEQ ID NO:1) and deduced amino acid (SEQ ID NO:2) sequences of Manduca Acheron. The nucleotide sequence does not contain the 5′-UTR or the translation initiation codon. The termination signal (TAG) is at site 1186 (boldface underlined) and the 3′UTR consists of 1061 bp with the polyadenylation signal at position 2230 (underlined). The protein sequence is partial and consists of 395 amino acids. It contains the LA domain (boxed), the Acheron motifs (dark shaded) and a putative bipartite nuclear localization signal (light shaded). The RNA binding domain (RBD) is boxed with dotted line. A potential amidation site at position 354 is double underlined.

FIG. 9 is a sequence listing showing the cDNA sequence (SEQ ID NO:3) and deduced amino acid sequence (SEQ ID NO:4) of human Acheron (hAch). The nucleotide sequence contains a presumably truncated 5′-UTR (70 bp), the translation initiation codon (in boldface) within a Kozak consensus sequence (underlined), the termination signal at site 1474 (boldface underlined) and the 3′-UTR with the polyadenylation signal and the poly (A+) site (both underlined). The open reading frame consists of 1473 nucleotides (SEQ ID NO:5) and encodes a protein of 491 amino acids. The La domain is boxed and the La-1, La-2 and La-3 motifs are underlined with dots. The highly conserved Acheron motifs are shaded (SEQ ID NOs:11, 12, and 13). The non-canonical RNA binding domain (SEQ ID NO:14) is boxed with dotted line. The putative nuclear localization signal (SEQ ID NO:16) is underlined with a thick line, the potential nuclear export signal is in boldface italics and underlined within the RBD. The putative amidation site (SEQ ID NO:15) is double underlined. The 3×SP repeats are double boxed. Exon junctions are shown.

FIG. 10 is a sequence alignment of Acheron proteins from human (SEQ ID NO: 4), mouse (SEQ ID NO:7), fly (SEQ ID NO:21) and moth (SEQ ID NO:2). Conserved amino acid residues are presented in black box shading, While conservative amino acid substitutions are depicted in gray box shading. The Acheron motifs I, II, and III are underlined. La motifs I, II, III and RBD (RNA binding domain) are shown. D. melanogaster Acheron protein is N- and C-terminally truncated. Gaps are introduced for optimal alignment.

### DETAILED DESCRIPTION

The present invention is based, in part, on the study of a model system based on the death of the intersegmental muscles of the tobacco hawk moth Manduca sexta, and the discovery of a novel death-associated gene termed Acheron (Ach). Moth (mAch) and human Acheron (hAch) share 31% identity and 40% similarity. Inhibition of Ach activity in a myoblast cell line reduces differentiation and apoptosis, while overexpression of Ach leads to increased levels of apoptosis. Acheron translocates to the nucleus in EGF-sensitive breast cancer cells in response to treatment with a mitogen, e.g., EGF. Furthermore, translocation of Acheron to the nucleus of rhabdomyosarcoma (RMS)-derived cells is associated with increased oncogenicity and metastatic potential. Thus, modulation of hAch activity, e.g., modulation of transcription, translation, post-translational modification, or translocation of Acheron, is useful in methods to increase apoptosis (in neoplastic cells, for example), and in methods to decrease apoptosis (for example, in conditions associated with cellular degeneration, or in cell-transplant procedures, including the transplantation of cells, including cells also expressing other, non-Ach genes for cell-based genetic therapies). Acheron also influences the differentiation of cells, thus making it useful for differentiation of tumor cells. Once cells, including tumor cells, exit the cell cycle and differentiate, their potential to undergo inappropriate mitosis or migration is reduced.

In some aspects, the invention provides methods for using Acheron as a screen for therapeutic agents that will affect apoptosis, e.g., by assaying binding to or effects on Acheron activity. As used herein, an “Acheron activity,” “biological activity of Acheron” or “functional activity of Acheron,” refers to an activity exerted by an Acheron protein, polypeptide, or nucleic acid molecule on, e.g., an Acheron-responsive cell, e.g., a cell expressing Acheron and/or the epidermal growth factor receptor (EGF-R), e.g., a myotube, myoblast, oligodendrocyte or other neural or muscle-derived cell, or tumor cell, or on an Acheron substrate, e.g., a protein substrate, e.g., CASK-C, as determined in vivo or in vitro. As one example, an Acheron activity can be modulation of apoptosis or differentiation. In one embodiment, an Acheron activity is a direct activity, such as an association with an Acheron target molecule. A “target molecule” or “binding partner” is a molecule with which an Acheron protein binds or interacts, e.g., Ariadne, parkin, or CASK-C. An Acheron activity can also be an indirect activity, e.g., a cellular signaling activity mediated by interaction of the Acheron protein with an Acheron binding partner. Thus, a modulator of Acheron activity can affect Acheron transcription, translation, post-translational modification, or translocation.

While the components of the execution phase of apoptosis have been defined, much less is known about the signal transduction pathways that activate this program in a lineage-specific manner. To identify these potential regulatory molecules, molecular techniques were used to screen for death-associated genes from the intersegmental muscles (ISMs) of the hawk moth Manduca sexta. The ISMs are composed of giant (˜5 mm long) fibers that die and disappear during a 30-hour period at the end of metamorphosis in response to endocrine cues.

The ISMs of Manduca become committed to die on day 17 of pupal-adult development and begin to actively die late the next day coincident with the emergence of the adult moth from the overlying pupal cuticle. A day 18 ISM cDNA library was screened for transcripts that were up-regulated in condemned cells. Using a differential cloning strategy, the moth Acheron (Genbank Acc# AF443827; SEQ ID NO:1) gene was identified based on a cDNA isolated in this screen that is dramatically up-regulated coincident with the commitment of the ISMs to die. The amino acid sequence is shown in SEQ ID NO:2.

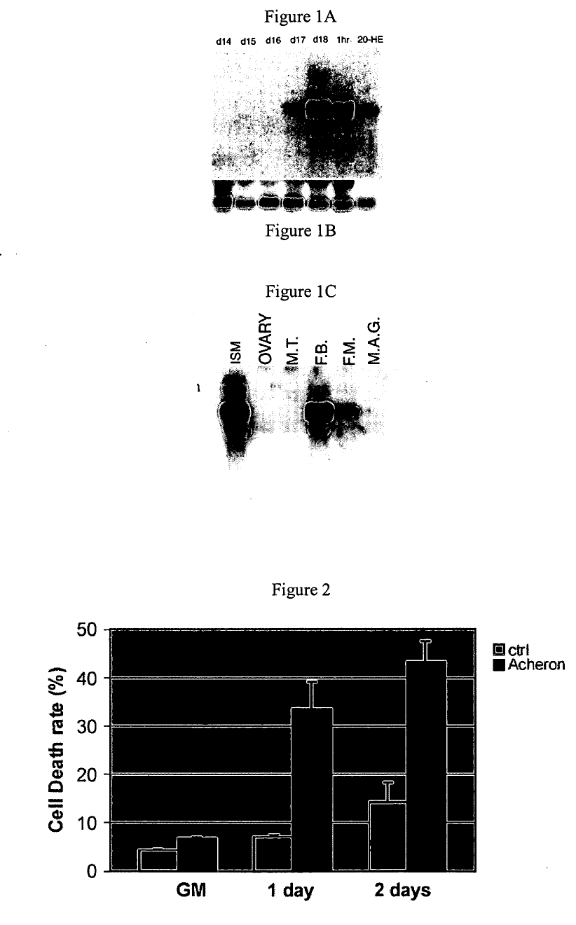

Northern blot analysis demonstrated that Acheron mRNA was undetectable in the ISMs until day 17 and then remained elevated throughout the initiation of death following adult emergence (3 and 5 hours post-emergence; FIG. 1A). Injection of day 17 animals with the insect molting hormone 20-hydroxyecdysone (20-HE), which delays the timing of ISM death, reduced the accumulation of Acheron mRNA (20-HE; FIG. 1A). To insure that elevations in Acheron expression were correlated with the commitment of the ISMs to die rather than just changes in circulating hormones, Acheron mRNA was examined in a variety of day 18 moth tissues including flight muscle, male sexual accessory gland, ovary, Malpighian tubules, and fat body. Acheron mRNA was most abundantly expressed in the ISMs (FIG. 1C; the presence of a low abundance, higher molecular weight transcript in the ISMs may reflect unprocessed message, alternative splicing or incomplete RNA denaturation). Acheron transcripts were also detected in fat body and to a lesser extent in flight muscle, but not in the other tissues examined. Since the ovary is composed predominantly of unfertilized oocytes, Acheron is not likely to be a maternal transcript.

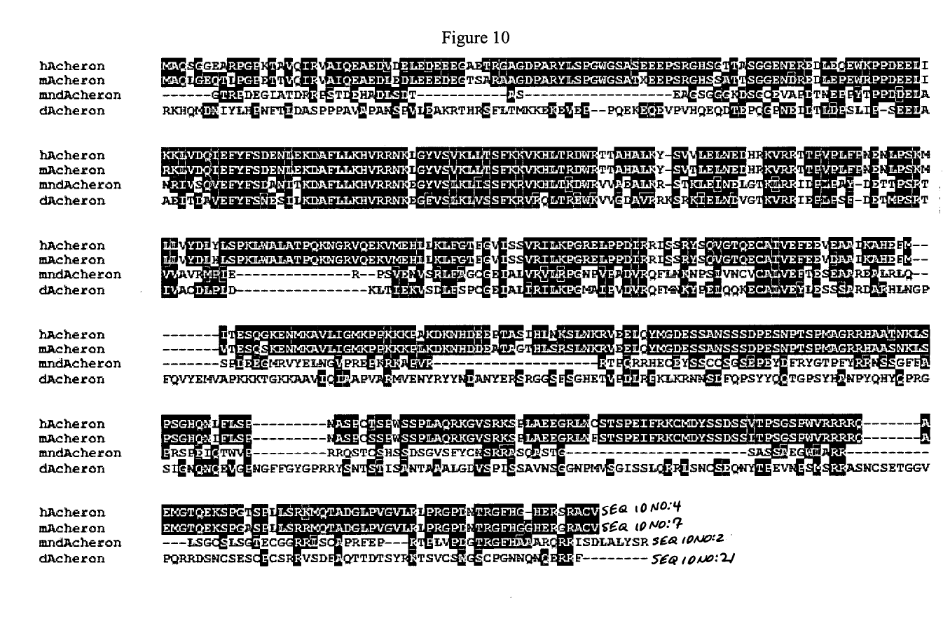

Database analysis revealed a human EST that shares 59% identity and 68% similarity over 86 amino acids with Manduca Acheron. Using the EST as probe, a human hippocampus cDNA library was screened and the human homolog of Acheron was isolated and the 5′ end region containing the translation initiation codon was cloned by inverse RT-RCR. The full-length cDNA sequence (Acc# AF443829; SEQ ID NO:3) has a total length of 2056 bp and encodes a protein of 491 amino acids long with a predicted molecular mass of 55 KDa. Database analysis revealed a Drosophila Acheron homolog (Acc # NP_{—}610964), and a mouse Acheron homolog cDNA clone (Acc#: AK017372; SEQ ID NO:6) isolated from a cDNA library generated from mRNA isolated from the head of 6 day old neonatal mice. Human and mouse Acheron proteins (SEQ ID NOs: 4 and 7, respectively) share 90% identity and 94% similarity overall, and each displays about 31% identity and 40% similarity to Manducan Acheron. The Drosophila homolog shows 31% identity and 46% similarity over 415 amino acids with the human protein. A sequence alignment of the Acheron proteins from human, mouse, fly and moth is shown in FIG. 10; using this alignment, one of skill in the art would be able to determine additional consensus sequences.

A search of the databases with the human Acheron amino acid sequence as a query sequence showed identity to the hypothetical human protein FLJ 1196 (AK002058), encoded by a cDNA isolated from a human placental cDNA library. This sequence contains one minor translational discrepancy at amino acid 103 (Y103C) with human Acheron. Human Acheron is also identical to the hypothetical partial human protein XM_{—}007678 and to the complete human proteins AAH06082.1 (BC006082) and AAH09446.1 (BC009446) isolated from rhabdomyosarcoma cells. There are 5 nucleotide differences between the FLJ 1196 (“FLJ”) and hAcheron as shown in SEQ ID NO:3 and 5 (hAch, Acc#AF443829): 1. FLJ 299t vs. hAch 298c; 2. FLJ 379g vs. hAch 378a; 3. FLJ 734t vs. hAch 733c; 4. FLJ 845c vs hAch 844 t; 5. FLJ 1429g vs. hAch 1428c. The second difference (FLJ 379g vs. hAch 378a) results in a change in the amino acid sequence, residue 103, which is Cys in FLJ, is Tyr in hAch.

Further analysis of human Acheron amino acid sequence revealed the presence of a number of functional domains, referred to herein as “Acheron functional domains. For example, the protein contains an N-terminal highly conserved La (Lupus Antigen) domain (ProDom 004143) spanning a region of 71 amino acids between 99-171 and consisting of the La-1 (99-116 aa, 61% identity to the authentic human La protein), La-2 (125-140 aa, 19% identity) and La-3 (156-171 aa, 50% identity) motifs. Thus, Acheron is highly related to the La (Lupus antigen) protein. La proteins serve a number of roles in cellular function and gene expression; a description of these properties can be found in the following review articles: Wolin and Cedervall, Annu. Rev. Biochem. 71:375-403 (2002); Maraia and Intine, Gene Expr. 10(1-2):41-57 (2001).

From insect to mammals, all Acheron proteins display extreme conservation within the La domain region with 100% identity over 13 amino acids at position 111-123 between La-1 and La-2 motifs, termed “Acheron motif I” (KDAFLLKHVRRNK; SEQ ID NO:11) (FIGS. 6 and 10). Two additional highly conserved motifs within the RNA binding domain were found, termed “Acheron motif II” ([V/I]-R-[V/I]-L-[K/R]-P-G; SEQ ID NO:12) at position 230-236 and “Acheron motif III” (C-A-[I/L]-V-E-[F/Y]; SEQ ID NO:13) at position 258-263.

Based on the properties of La, and the structural similarities between Acheron and the La proteins, it is reasonable to speculate that Acheron could also participate in some or all of the same activities as the La proteins. Therefore, Acheron may participate in the following processes:
1) RNA processing
2) RNA chaperone
3) regulation of viral gene expression
4) regulation of mRNA translation
5) control of RNA stability, including tRNA, rRNA and mRNA
6) RNAi or siRNA function
7) RNA splicing

In addition, human Acheron contains other Acheron functional domains including several putative N-linked glycosylation sites (317-320, 337-340, and 405-408); a putative tyrosine sulfation site at 96-110; putative cAMP-and cGMP-dependent protein kinase phosphorylation sites at 168-171 and 244-247; a number of putative protein kinase C phosphorylation sites at 128-130, 134-136, 194-196, 229-231, 247-249, 358-360, 393-395, and 455-457; putative casein kinase II phosphorylation sites at 4-7, 56-59, 58-61, 72-75, 338-341, 340-343, and 408-411; putative tyrosine kinase phosphorylation sites at 41-49 and 322-329; putative N-myristoylation sites at 68-73, 225-230, 254-259, and 463-468; an imperfect RNA binding domain (RBD; SEQ ID NO:14), also known as RNA recognition motif (RRM), between amino acids 184-296; a putative amidation site (AGRR; SEQ ID NO:15) at amino acid positions 351-354; a number of putative tyrosine and serine/threonine phosphorylation sites; a possible nuclear localization signal (PKKKPAK; SEQ ID NO:16) at amino acid position 297-103; a potential nuclear export signal (LLVYDLYL; SEQ ID NO:17) at position amino acids 186-193; and a 3×SP repeats at position 376-385 found in the transcription factors of the NF-AT family (see FIG. 6).

The genomic structure of the human Acheron gene was determined. The human Acheron gene spans a region of 22,590 bp of the human genome and its coding region is distributed over 3 exons. The 5′ UTR sequence contains 70 bp and no additional sequence for this region is currently present in public databases. Minor nucleotide sequence discrepancies were observed between our sequence and those in the databases, most notably in exon 1. Exon 1 with part of the flanking intron 1 sequence have a high GC content (80%) suggesting a possible role as a CpG regulatory island.

Human EST database analysis using the genomic human Acheron sequence as a query revealed three additional putative exons between exon 1 and exon 2, suggesting the existence of alternatively spliced isoforms.

The chromosomal localization of human Acheron gene was determined by radiation hybrid mapping using the Genebridge 4 panel of 93 radiation rodent hybrid clones of the whole human genome and analyzing the results with the RHMAPPER (version 1.22) program (Whitehead Institute/MIT Center for Genome Research). According to this analysis, the human Acheron gene is located on Chr 15, 1.71 cR distal to Whitehead framework marker WI-6247 with lod >3.0 within the microsatellites interval D15S216-D15S160. This interval is mapped in the q22.3-q23 region of chromosome 15 and is localized within the extended 9 cM interval cen—D15S125-D15S114-qter. Further analysis also narrowed the humanAcheron gene location within the interval D15S197-D15S160, a region less than 1 cM long within the 2cM BBS4 locus, placing it at the same position as the framework marker WI-19667 (STS-T15623), 253.46 cR from the top of chromosome 15 in the GB4 radiation hybrid map. Comparison of the WI-19667 sequence with the human Acheron cDNA showed 100% identity to a region of the 3′-UTR of human Acheron transcript and to the human Acheron PCR product used in the radiation hybrid mapping.

Three common synonymous polymorphisms were found in exon 3: a T>C at residue 661 changing TTC to TTT (Phe221Phe); a T>C at residue 772 changing TGT to TGC (Cys258Cys); and a C>T at residue 1362 changing CTC to CTT (Leu454Leu). A heterozygous nucleotide substitution resulting in a missense change was found in one individual in exon 3, resulting in a His484Asp substitution, which was presumed to be a rare variant, since no change was found in the other allele. SSCP analysis was also performed in normal individuals as a control and samples exhibiting shifts in the SSCP gels were sequenced.

Human Acheron is widely expressed in human adult and fetal tissues, including total fetus 8-9 weeks post-conception (p.c.), fetal heart and lung 19 weeks p.c., fetal liver and spleen 20 weeks p.c. and fetal brain of 20, 24, 26 weeks p.c. Expression has also been found in infant and 15 weeks postnatal brain. In adults, human Acheron transcript expression has been observed in bones, bone marrow stroma cells, kidney, prostate, testis, post-menopausal ovary, uterus, pregnant uterus, placenta, colon, pancreatic islets, and lymph nodes. It is also expressed in the hippocampus and hypothalamus of the brain and in dorsal root ganglia of the peripheral nervous system.

Human Acheron mRNA is also expressed in neoplastic tissues including metastasis-positive ovarian tumors of different types such as mixed Mallerian tumor, papillary serous adenocarcinoma, clear cell and spindle cell carcinoma. Expression has also been observed in skeletal muscle rhabdomyosarcoma, clear cell adenocarcinoma of the kidney, pancreas, mammary gland and colon metastatic adenocarcinomas, primary and metastatic Wilm's tumor, germ cell tumors, lung carcinoid, uterus well-differentiated endometrial adenocarcinoma, uterus leiomyosarcoma, melanoma, nasopharyngeal and adrenal gland cortex carcinoma. Brain tumors, such as anaplastic oligodendroglioma, glioblastoma, and neuroblastoma, are also among the neoplasms that express human Acheron transcript.

Quantitative evaluation of gene expression by SAGE analysis (Velculescu et al., Science 270(5235):484-7 (1995)) revealed high expression in cerebellum, brain white matter, ovary normal surface epithelium, glioblastoma multiforme cell line H566 (telomerase positive), ovary carcinoma pooled cell lines and normal mammary gland epithelial organoids.

The expression patterns of the rat Acheron homologue were evaluated in sagittal sections of an E16 rat embryo and a coronal section through the head of a P1 neonatal rat pup. Clear staining was seen in the embryonic nervous system, and in the cortex, hippocampus, amygdala, and thalamus of the neonatal brain. Expression in the cortex at this stage of embryogenesis indicates a role in neuronal migration and differentiation. This suggests a role for Acheron in neurogenesis and neurodevelopmental defects.

Ectopic expression of hAch in mouse C2C12 myoblasts blocks Myf5 and Bcl-2 expression and greatly reduces the survival of mononucleated reserve cells in differentiation medium. In contrast, dominant-negative or antisense hAch blocks MyoD expression, myotube formation and apoptosis, resulting in almost pure populations of “reserve cells” (see Examples, below), which are mononucleated cells that share many characteristics with skeletal muscle satellite cells, including quiescence, self-renewal, and the ability to generate multinucleated myotubes. Taken together, these data suggest that the phylogenetically-conserved Acheron protein may mediate a key branch point in myogenesis by controlling differentiation and death.

To investigate the mechanisms by which Acheron regulates differentiative decisions, a yeast two-hybrid screen was performed with Acheron as the bait and a mouse embryonic day 17 cDNA library as the prey. About 4.8×10⁶ transformants were screened, out of which two Acheron-binding partners were identified. One is Ariadne, a RING finger protein with structural and functional homology to the parkin protein, which is encoded by a gene that is believed to be responsible for Autosomal Recessive Juvenile Parkinsonism. RING finger proteins function as ubiquitin E3 ligases to target specific substrates for ubiquitin-proteasome-dependent degradation, and thus Ariadne may play a crucial role in regulating levels of Acheron by targeting Acheron for degradation.

A second clone encoded a novel isoform of the calcium/calmodulin-dependent serine protein kinase (CASK) gene family that contains an N-terminal CaM kinase II domain and C-terminal membrane-associated guanylate kinase (MAGUK) domain. CASK is a homolog of the C. elegans lin-2 gene that controls major lineage-specific decisions in worms and mammals. While the novel CASK gene encodes a protein that shares high sequence identity with the two other previously described mammalian CASKs, it represents an independent gene, now named CASK-C (SEQ ID NOs:9 and 10). CASK functions as a transcription factor, but does not have a nuclear localization domain. The Acheron protein does contain this targeting motif and can be driven into the nucleus when cells are treated with growth factors, such as EGF. As one theory, Acheron may act as a shuttle, translocating CASK-C to the nucleus (see Examples 10-13).

The interaction between Acheron and CASK-C is specific and has been confirmed in yeast two hybrid assays by switching the bait and prey sequences, as well as by GST-pull-down assays (see Examples 10-13). These and other assays indicate that the N-terminus of Acheron interacts specifically with the CaM kinase II domain of CASK-C. As one theory, not meant to be limiting, the ability of Acheron to regulate differentiative decisions in myoblasts may be mediated by CASK-C.

Acheron Polynucleotides and Polypeptides

The invention is based, in part, on the discovery and characterization of a gene referred to herein as Acheron (also referred to as “Ach”). The nucleotide sequence of a cDNA encoding the human isoform of Acheron is SEQ ID NO:3, and the deduced amino acid sequence of a human Acheron polypeptide is SEQ ID NO:4. In addition, the nucleotide sequence of the coding region is SEQ ID NO:5.

The human Acheron sequence (SEQ ID NO:3), which is approximately 2056 nucleotides long including untranslated regions, contains a predicted methionine-initiated coding sequence of about 1476 nucleotides, including the termination codon (nucleotides indicated as coding of SEQ ID NO:3; the coding sequence is SEQ ID NO:5). The coding sequence encodes a 491 amino acid protein (SEQ ID NO:4). Structural analysis of Acheron failed to identify any obvious catalytic domains. hAch contains a highly conserved N-terminal La (Lupus antigen) motif (ProDom 004143, amino acids 99-171 of SEQ ID NO:4 in human and mouse), three La-like motifs, an imperfect RNA binding domain, and a putative nuclear localization signal. Database analysis and phylogenetic tree construction revealed that Acheron proteins are highly conserved and structurally related to La proteins, but define a new subfamily.

The Acheron protein, fragments thereof, and derivatives and other variants of the sequence in SEQ ID NO:4 thereof are collectively referred to as “polypeptides or proteins of the invention” or “Acheron polypeptides or proteins.” Nucleic acid molecules encoding such polypeptides or proteins are collectively referred to as “nucleic acids of the invention” or “Acheron nucleic acids.” “Acheron molecules” refers to Acheron nucleic acids and polypeptides.

As used herein, the term “nucleic acid molecule” includes DNA molecules (e.g., a cDNA or genomic DNA) and RNA molecules (e.g., mRNA or siRNA, e.g., dsRNA) and analogs of the DNA or RNA generated, e.g., by the use of nucleotide analogs. The nucleic acid molecule can be single-stranded or double-stranded. In one embodiment, the nucleic acid is double-stranded DNA. The nucleic acid can be complementary to the sequence of SEQ ID NO:3 or 5, e.g., an antisense nucleic acid. Thus the invention includes Acheron nucleic acids including variants, fragments, antisense nucleic acid molecules, ribozymes, small interfering ribonucleic acids (siRNA), and modified acheron nucleic acid molecules.

The term “isolated or purified nucleic acid molecule” includes nucleic acid molecules that are separated from other nucleic acid molecules that are present in the natural source of the nucleic acid. For example, with regards to genomic DNA, the term “isolated” includes nucleic acid molecules that are separated from the chromosome with which the genomic DNA is naturally associated. An “isolated” nucleic acid is typically free of sequences that naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of 5′ and/or 3′ nucleotide sequences that naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term “hybridizes under stringent conditions” describes conditions for hybridization and washing. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. Stringent hybridization conditions are hybridization in 6×SSC at about 45° C., followed by one or more washes in 0.2×SSC, 0.1% SDS at 65° C. Typically, an isolated nucleic acid molecule of the invention that hybridizes under stringent conditions to the sequence of SEQ ID NO:3 or SEQ ID NO:5, corresponds to a naturally-occurring nucleic acid molecule (or the complement thereof).

As used herein, a “naturally-occurring” nucleic acid molecule refers to an RNA or DNA molecule having a nucleotide sequence that occurs in nature (e.g., encodes a natural or wild type protein).

As used herein, the terms “Acheron gene” and “recombinant Acheron gene” refer to nucleic acid molecules that include an open reading frame encoding an Acheron protein, e.g., a mammalian Acheron protein, and can further include non-coding regulatory sequences, and introns.

An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of Acheron protein having less than about 10% (by dry weight), of non-Acheron protein (also referred to herein as a “contaminating protein”), or of chemical precursors or non-Acheron chemicals. When the Acheron protein or fragment thereof is recombinantly produced, it is also typically substantially free of culture medium, i.e., culture medium represents less than about 10% of the volume of the protein preparation. The invention includes isolated or purified preparations of at least 0.01 milligrams in dry weight.

A “non-essential” amino acid residue is a residue that can be altered from the wild-type sequence of Acheron (e.g., the sequence of SEQ ID NO:4) without abolishing or substantially altering a biological activity, whereas an “essential” amino acid residue results in such a change.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an Acheron protein can be replaced with another amino acid residue from the same side chain family. Alternatively, in another embodiment, mutations can be introduced randomly along all or part of an Acheron coding sequence, such as by saturation mutagenesis, and the resultant mutants can be screened for Acheron biological activity to identify mutants that retain activity. Following mutagenesis of SEQ ID NO:3 or SEQ ID NO:5, the encoded protein can be expressed recombinantly and the activity of the protein can be determined.

As used herein, a “biologically active portion” of an Acheron protein includes a fragment of an Acheron protein that has at least one biological activity of the full length protein, e.g., at least 25%, e.g., about 35%, 50%, 65%, 80%, 90%, or 100%, of at least one biological activity of the full length protein. Biologically active portions of an Acheron protein include peptides comprising amino acid sequences sufficiently homologous to or derived from the amino acid sequence of the Acheron protein, e.g., the amino acid sequence shown in SEQ ID NO:4, that include fewer amino acids than the full length Acheron proteins, and exhibit at least one activity of an Acheron protein. Typically, biologically active portions comprise a domain or motif with at least one activity of the Acheron protein, e.g., regulation of apoptosis and/or differentiation. A biologically active portion of an Acheron protein can be a polypeptide that is, for example, 10, 25, 50, 100, 200 or more amino acids in length. Biologically active portions of an Acheron protein can be used as targets for developing agents that modulate an Acheron mediated activity, e.g., regulation of apoptosis or differentiation.

Calculations of homology or sequence identity between sequences (the terms are used interchangeably herein) can be performed using methods known in the art, including as follows:

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a sequence aligned for comparison purposes is at least 60% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. The percent identity between two amino acid sequences can be determined a Blossum 62 scoring matrix with a gap penalty of 12, a gap extend penalty of 4, and a frameshift gap penalty of 5.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to Acheron nucleic acid molecules of the invention. BLAST protein searches can be performed with the XBLAST program, score=50, wordlength=3 to obtain amino acid sequences homologous to Acheron protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See the world wide web at ncbi.nlm.nih.gov.

Acheron polypeptides of the invention have an amino acid sequence substantially identical to the amino acid sequence of SEQ ID NO:4. The term “substantially identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain or common functional activity. For example, amino acid or nucleotide sequences that contain a common structural domain having at least about 95% identity are defined herein as sufficiently or substantially identical.

“Misexpression or aberrant expression,” as used herein, refers to a non-wild type pattern of gene expression, at the RNA or protein level. It includes expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of subcellular localization; a pattern of expression that differs from a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

“Subject,” as used herein, can refer to a mammal, e.g., a human, or to an experimental or animal or disease model. The subject can also be a non-human animal, e.g., a veterinary subject, e.g., horse, cow, pig, goat, cat, dog, mouse, rat or other domestic animal. The subject can also be an insect, e.g., a hawk moth.

A “purified preparation of cells” as used herein refers to, in the case of animal cells, an in vitro preparation of cells and not an entire intact animal. In the case of cultured cells or microbial cells, it consists of a preparation of at least 10% and more typically 50% of the subject cells.

Isolated Nucleic Acid Molecules

In one aspect, the invention provides an isolated or purified nucleic acid molecule that encodes an Acheron polypeptide described herein, e.g., a full length Acheron protein or a fragment thereof, e.g., a biologically active portion of Acheron protein or other functional fragment, e.g., dominant negative fragments. Also included is a nucleic acid fragment suitable for use as a hybridization probe, which can be used, e.g., to identify a nucleic acid molecule encoding an Acheron polypeptide, e.g., an Acheron mRNA, and fragments suitable for use as primers, e.g., PCR primers for the amplification or mutation of nucleic acid molecules.

In one embodiment, an isolated Acheron nucleic acid molecule includes the nucleotide sequence shown in SEQ ID NO:3 or SEQ ID NO:5, or a portion of any of these nucleotide sequences. In one embodiment, the nucleic acid molecule includes sequences encoding the human Acheron protein (i.e., “the coding region” of SEQ ID NO:3, as shown in SEQ ID NO:5), as well as 5′ untranslated sequences. Alternatively, the nucleic acid molecule can include only the coding region of SEQ ID NO:3 (e.g., SEQ ID NO:5) and, e.g., no flanking sequences that normally accompany the subject sequence. In another embodiment, the nucleic acid molecule encodes a sequence corresponding to an N-terminally truncated fragment of the protein including from about amino acid 34 to amino acid 492 of SEQ ID NO:4 (also referred to herein as truncated Acheron, or “tAch”). In another embodiment, an isolated nucleic acid molecule of the invention includes a nucleic acid molecule that is a complement of the nucleotide sequence shown in SEQ ID NO:3 or SEQ ID NO:5, or a portion of any of these nucleotide sequences. In other embodiments, the nucleic acid molecule of the invention is sufficiently complementary to the nucleotide sequence shown in SEQ ID NO:3 or SEQ ID NO:5, such that it can hybridize to the nucleotide sequence shown in SEQ ID NO:3 or 5, thereby forming a stable duplex.

In one embodiment, an isolated Acheron nucleic acid molecule includes a nucleotide sequence that is at least about 95%, 96%, 97%, 98%, 99% or more homologous to the entire length of the nucleotide sequence shown in SEQ ID NO:3 or SEQ ID NO:5, or a portion thereof.

Acheron Nucleic Acid Fragments

The nucleic acid molecules of the invention include portions or fragments of the nucleic acid sequences of SEQ ID NO:3 or 5. For example, such fragments can be used as a probe or primer or to encode a portion of an Acheron protein, e.g., an immunogenic or biologically active portion of an Acheron protein. The nucleotide sequence determined from the cloning of the Acheron gene allows for the generation of probes and primers designed for use in identifying and/or cloning other Acheron family members, or fragments thereof, as well as Acheron homologues, or fragments thereof, from other species.

In another embodiment, a nucleic acid includes a nucleotide sequence that includes part, or all, of the coding region and extends into either (or both) the 5′ or 3′ noncoding region. Other embodiments include a fragment that includes a nucleotide sequence encoding an amino acid fragment described herein. Nucleic acid fragments can encode a specific domain or site described herein or fragments thereof, particularly fragments thereof that are at least 100, 200, 300, or 400 amino acids in length. Fragments also include nucleic acid sequences corresponding to specific amino acid sequences described herein or fragments thereof. For example, a fragment can comprise, e.g., those nucleotides of SEQ ID NO:3 or 5 that encode amino acids 34-491 of human Acheron (SEQ ID NO:4), e.g., an N-terminally truncated form of Acheron that acts as a dominant negative to reduce Acheron activity. Nucleic acid fragments should not to be construed as encompassing those fragments that may have been disclosed prior to the invention.

A nucleic acid fragment can include a sequence corresponding to an Acheron functional domain, region, or functional site described herein. A nucleic acid fragment can also include one or more Acheron functional domain, region, or functional site described herein. Thus, for example, an Acheron nucleic acid fragment can include a sequence corresponding to a La domain.

Acheron probes and primers are provided. Typically a probe/primer is an isolated or purified oligonucleotide. The oligonucleotide typically includes a region of nucleotide sequence that hybridizes under stringent conditions to at least about 7, 12 or 15, 20 or 25, 30, 35, 40, 45, 50, 55, 60, 65, or 75 consecutive nucleotides of a sense or antisense sequence of SEQ ID NO:3 or SEQ ID NO:5, or of a naturally occurring allelic variant or mutant of SEQ ID NO:3 or SEQ ID NO:5.

In one embodiment, the nucleic acid is a probe that is at least 10, 12, or 15, and less than 200, 100, or 50, base pairs in length. It should be identical, or differ by 1, or less than 5 or 10 bases, from a sequence disclosed herein. If alignment is needed for this comparison the sequences should be aligned for maximum homology. “Looped” out sequences from deletions or insertions, or mismatches, are considered differences.

A probe or primer can be derived from the sense or anti-sense strand of a nucleic acid that encodes one or more portions of hAch, e.g., the first (N-terminal) 34 amino acids, one or more of the La domains, or the potential localization or RNA-binding domains.

In another embodiment a set of primers is provided, e.g., primers suitable for use in a PCR, which can be used to amplify a selected region of an Acheron sequence, e.g., a domain, region, site or other sequence described herein. The primers should be at least 10, 12, 15, 20, 25 or 50 base pairs in length and less than 100, or less than 200, base pairs in length. The primers should be identical, or differ by one base from a sequence disclosed herein or from a naturally occurring variant.

A nucleic acid fragment can encode an epitope-bearing region of a polypeptide described herein.

A nucleic acid fragment encoding a “biologically active portion of an Acheron polypeptide” can be prepared by isolating a portion of the nucleotide sequence of SEQ ID NO:3 or 5, which encodes a polypeptide having an Acheron biological activity (e.g., the biological activities of the Acheron proteins as described herein), expressing the encoded portion of the Acheron protein (e.g., by recombinant expression in vitro), and assessing the activity of the encoded portion of the Acheron protein. A nucleic acid fragment encoding a biologically active portion of an Acheron polypeptide can comprise a nucleotide sequence that is greater than 200, 300, 400 or more nucleotides in length.

In some embodiments, a nucleic acid includes a nucleotide sequence that is about 300, 400, 500, 600, 700, 800, 900, 1000, 1100, 1200, 1300 or more nucleotides in length and hybridizes under stringent hybridization conditions to a nucleic acid molecule of SEQ ID NO:3 or SEQ ID NO:5 (or the complement thereof).

Acheron Nucleic Acid Variants

The invention further encompasses nucleic acid molecules that differ from the nucleotide sequence shown in SEQ ID NO:3 or SEQ ID NO:5. Such differences can be due to degeneracy of the genetic code, and result in a nucleic acid that encodes the same Acheron proteins as those encoded by the nucleotide sequence disclosed herein. In one embodiment, an isolated nucleic acid molecule of the invention has a nucleotide sequence encoding a protein having an amino acid sequence that differs by at least 1, but less than 5, 10, 20 or 25 amino acid residues from that shown in SEQ ID NO:4. If alignment is needed for this comparison the sequences should be aligned for maximum homology. “Looped” out sequences from deletions or insertions, or mismatches, are considered differences.

Nucleic acids of the invention can be chosen for having codons that are preferred for a particular expression system. For example, the nucleic acid can be one in which at least one or more codons, typically at least 10% or 20% of the codons, have been altered such that the sequence is optimized for expression in E. Coli, yeast, human, insect, or CHO cells.

Nucleic acid variants can be naturally occurring, such as allelic variants (same locus), homologs (different locus), and orthologs (different organism), or can be non-naturally occurring. Non-naturally occurring variants can be made by mutagenesis techniques, including those applied to polynucleotides, cells, or organisms. The variants can contain nucleotide substitutions, deletions, inversions, and insertions. Variation can occur in either or both the coding and non-coding regions. The variations can produce both conservative and non-conservative amino acid substitutions (as compared in the encoded product).

In one embodiment, the nucleic acid differs from that of SEQ ID NO:1 or 3, or the sequence in ATCC Accession Number AF443829, e.g., by at least one nucleotide but less than 10, 20, 30, or 40 nucleotides; at least one nucleotide but less than 1%, 5%, or 10% of the nucleotides in the subject nucleic acid. If necessary for this analysis the sequences should be aligned for maximum homology. “Looped” out sequences from deletions or insertions, or mismatches, are considered differences.

Orthologs, homologs, and allelic variants can be identified using methods known in the art. These variants comprise a nucleotide sequence encoding a polypeptide that is at least about 65%, about 70-75%, about 80-85%, or at least about 90-95% or more identical to the nucleotide sequence shown in SEQ ID NOs:3 or 5 or a fragment of these sequences. Such nucleic acid molecules can readily be identified as being able to hybridize under stringent conditions, to the nucleotide sequences shown in SEQ ID NO:3 or 5 or a fragment of the sequence or the complement thereof. Nucleic acid molecules corresponding to orthologs, homologs, and allelic variants of the Acheron cDNAs of the invention can further be isolated by mapping to the same chromosome or locus as the Acheron gene. Variants can include those that are correlated with apoptosis or differentiation.

Allelic variants of Acheron, e.g., human Acheron, include both functional and non-functional proteins. Functional allelic variants are naturally occurring amino acid sequence variants of the Acheron protein within a population that have the ability to affect apoptosis or differentiation. Functional allelic variants will typically contain only conservative substitution of one or more amino acids of SEQ ID NO:4, or substitution, deletion or insertion of non-critical residues in non-critical regions of the protein. Non-functional allelic variants are naturally-occurring amino acid sequence variants of the Acheron, e.g., human Acheron, protein within a population that do not have the ability to affect apoptosis or differentiation. Non-functional allelic variants will typically contain a non-conservative substitution, a deletion, or insertion, or premature truncation of the amino acid sequence of SEQ ID NO:4, or a substitution, insertion, or deletion in critical residues or critical regions of the protein. Such non-functional allelic variants may be useful, e.g., as dominant negatives or competitive inhibitors.

Moreover, nucleic acid molecules encoding other Acheron family members and, thus, which have a nucleotide sequence which differs from the Acheron sequences of SEQ ID NO:3 or SEQ ID NO:5 are intended to be within the scope of the invention.

Antisense Nucleic Acid Molecules, Ribozymes, Small Interfering Ribonucleic Acids (siRNA), and Modified Acheron Nucleic Acid Molecules

The invention also includes nucleic acid molecules that can be used to modify, e.g., enhance or inhibit, Acheron expression or activity. These include antisense, siRNA, ribozymes, and other modified nucleic acid molecules such as PNAs. These nucleic acids can be introduced into the cells for expression purposes (e.g., using a vector that expresses an antisense or siRNA that inhibits Acheron expression) or can be used more transiently, e.g., by treating the cells with isolated antisense or RNAi molecules. This has the advantage that the effects of inhibiting Acheron should be transient. Since Acheron inhibits both death and differentiation, this is desirable; as transient inhibition of Acheron activity or expression allows the cells to survive initially and then, over time, acquire the capacity to differentiate or fuse with other cells. Once either of these steps happen, they will activate survival programs and not need the benefits of Acheron. In addition, the cells should be able to undergo cell death as appropriate, alleviating long term concerns about implanting what are essentially immortalized cells into a host.

In another aspect, the invention features an isolated nucleic acid molecule that is an antisense strand of nucleotides that hybridizes to Acheron mRNA. An “antisense” nucleic acid can include a nucleotide sequence that is complementary to a “sense” nucleic acid encoding a protein, e.g., complementary to the coding strand of a double-stranded cDNA molecule or complementary to an mRNA sequence. The antisense nucleic acid can be complementary to an entire Acheron coding strand, or to only a portion thereof (e.g., all or part of the coding region of human Acheron corresponding to SEQ ID NO:5). In another embodiment, the antisense nucleic acid molecule is antisense to all or part of a “noncoding region” of the coding strand of a nucleotide sequence encoding Acheron (e.g., the 5′ and 3′ untranslated regions). Based upon the sequences disclosed herein, one of skill in the art can easily choose and synthesize any of a number of appropriate antisense molecules for use in accordance with the present invention. For example, a “gene walk” comprising a series of oligonucleotides of 15-30 nucleotides spanning the length of a FIAT nucleic acid can be prepared, followed by testing for inhibition of FIAT expression. Optionally, gaps of 5-10 nucleotides can be left between the oligonucleotides to reduce the number of oligonucleotides synthesized and tested.

An antisense nucleic acid can be designed such that it is complementary to the entire coding region of Acheron mRNA, but more typically is an oligonucleotide that is antisense to only a portion of the coding or noncoding region of Acheron mRNA. For example, the antisense oligonucleotide can be complementary to the region surrounding the translation start site of Acheron mRNA, e.g., between the −10 and +10 regions of the target gene nucleotide sequence of interest. The antisense oligonucleotide can correspond to all or part of nucleotides 97-1398 of SEQ ID NO: 5. The antisense oligonucleotide can target the regions of SEQ ID NO:3 or 5 that encode residues 1-33; residues 34-491; or all or part of one or more of the Acheron functional domains. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection). In some embodiments, the antisense nucleic acid is a morpholino oligonucleotide (see, e.g., Heasman, Dev. Biol. 243:209-14 (2002); Iversen, Curr. Opin. Mol. Ther. 3:235-8 (2001); Summerton, Biochim.

Biophys. Acta. 1489:141-58 (1999).

The antisense nucleic acid molecules of the invention are typically administered to a subject (e.g., by direct injection at a tissue site), or generated in situ such that they hybridize with or bind to cellular mRNA and/or genomic DNA encoding an Acheron protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter can be used.

In yet another embodiment, the antisense nucleic acid molecule of the invention is an α-anomeric nucleic acid molecule. An α-anomeric nucleic acid molecule-forms specific double-stranded hybrids with complementary RNA in which, contrary to the usual β-units, the strands run parallel to each other (Gaultier et al. Nucleic Acids. Res. 15:6625-6641 (1987). The antisense nucleic acid molecule can also comprise a 2′-o-methylribonucleotide (Inoue et al. Nucleic Acids Res. 15:6131-6148 (1987) or a chimeric RNA-DNA analogue (Inoue et al. FEBS Lett. 215:327-330 (1987).

In still another embodiment, an antisense nucleic acid of the invention is a ribozyme.

A ribozyme having specificity for an Acheron-encoding nucleic acid can include one or more sequences complementary to the nucleotide sequence of an Acheron cDNA disclosed herein (i.e., SEQ ID NO:3 or SEQ ID NO:5), and a sequence having known catalytic sequence responsible for mRNA cleavage (see U.S. Pat. No. 5,093,246 or Haselhoff and Gerlach, Nature 334:585-591 (1988). For example, a derivative of a Tetrahymena L-19 IVS RNA can be constructed in which the nucleotide sequence of the active site is complementary to the nucleotide sequence to be cleaved in an Acheron-encoding mRNA. See, e.g., Cech et al., U.S. Pat. No. 4,987,071; and Cech et al., U.S. Pat. No. 5,116,742. Alternatively, Acheron mRNA can be used to select a catalytic RNA having a specific ribonuclease activity from a pool of RNA molecules. See, e.g., Bartel and Szostak, Science 261:1411-1418 (1993). For example, a ribozyme can target a region of SEQ ID NO:3 or 5 that encodes one or more of residues 1-33; residues 34-491; or the Acheron functional domains.

Acheron gene expression can be inhibited by targeting nucleotide sequences complementary to the regulatory region of the Acheron (e.g., the Acheron promoter and/or enhancers) to form triple helical structures that prevent transcription of the Acheron gene in target cells. See generally, Helene, Anticancer Drug Des. 6:569-84 (1991); Helene Ann. N.Y. Acad. Sci. 660:27-36 (1992); and Maher Bioassays 14:807-15 (1992). The potential sequences that can be targeted for triple helix formation can be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or colorimetric.

An Acheron nucleic acid molecule can be modified at the base moiety, sugar moiety or phosphate backbone to improve the stability, hybridization, or solubility of the molecule. For example, the deoxyribose phosphate backbone of the nucleic acid molecules can be modified to generate peptide nucleic acids (see Hyrup B. et al. Bioorganic & Medicinal Chemistry 4: 5-23 (1996). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of Acheron nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of Acheron nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe supra).

In other embodiments, the oligonucleotide can include other appended groups such as peptides (e.g., for targeting host cell receptors in vivo), or agents facilitating transport across the cell membrane (see, e.g., Letsinger et al. Proc. Natl. Acad. Sci. USA 86:6553-6556 (1989); Lemaitre et al. Proc. Natl. Acad. Sci. USA 84:648-652 (1987); PCT Publication No. WO88/09810) or the blood-brain barrier (see, e.g., PCT Publication No. WO89/10134). In addition, oligonucleotides can be modified with hybridization-triggered cleavage agents (see, e.g., Krol et al. Bio-Techniques 6:958-976 (1988) or intercalating agents. (see, e.g., Zon, Pharm. Res. 5:539-549 (1988). To this end, the oligonucleotide can be conjugated to another molecule, (e.g., a peptide, hybridization triggered cross-linking agent, transport agent, or hybridization-triggered cleavage agent).

The invention also includes molecular beacon oligonucleotide primer and probe molecules having at least one region that is complementary to an Acheron nucleic acid of the invention, two complementary regions one having a fluorophore and one a quencher such that the molecular beacon is useful for quantitating the presence of the Acheron nucleic acid of the invention in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

RNA Interference

RNAi is a remarkably efficient process whereby double-stranded RNA (dsRNA, also referred to herein as siRNAs for small interfering RNAs or ds-siRNAs, for double-stranded small interfering RNAs) induces the sequence-specific degradation of homologous mRNA in animals and plant cells (Hutvagner and Zamore, Curr. Opin. Genet. Dev.:12, 225-232 (2002); Sharp, Genes Dev., 15:485-490 (2001)). In mammalian cells, RNAi can be triggered by 21-nucleotide (nt) duplexes of small interfering RNA (siRNA) (Chiu et al., Mol. Cell. 10:549-561 (2002); Elbashir et al., Nature 411:494-498 (2001)), or by micro-RNAs (mRNA), functional small-hairpin RNA (shRNA), or other dsRNAs that are expressed in vivo using DNA templates with RNA polymerase III promoters (Zeng et al., Mol. Cell 9:1327-1333 (2002); Paddison et al., Genes Dev. 16:948-958 (2002); Lee et al., Nature Biotechnol. 20:500-505 (2002); Paul et al., Nature Biotechnol. 20:505-508 (2002); Tuschl, T., Nature Biotechnol. 20:440-448 (2002); Yu et al., Proc. Natl. Acad. Sci. USA 99(9):6047-6052 (2002); McManus et al., RNA 8:842-850 (2002); Sui et al., Proc. Natl. Acad. Sci. USA 99(6):5515-5520 (2002).)

Accordingly, the invention includes such molecules that are targeted to an Acheron mRNA.

siRNA Molecules

The nucleic acid molecules or constructs of the invention include dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand. The dsRNA molecules of the invention can be chemically synthesized, or can be transcribed in vitro from a DNA template, or in vivo from, e.g., shRNA. The dsRNA molecules can be designed using any method known in the art, for instance, using the following protocol:
1. Beginning with the AUG start codon, look for AA dinucleotide sequences; each AA and the 3′ adjacent 16 or more nucleotides are potential siRNA targets. siRNAs taken from the 5′ untranslated regions (UTRs) and regions near the start codon (within about 75 bases or so) may be less useful as they may be richer in regulatory protein binding sites, and UTR-binding proteins and/or translation initiation complexes may interfere with binding of the siRNP or RISC endonuclease complex. Thus, in one embodiment, the nucleic acid molecules are selected from a region of the cDNA sequence beginning 50 to 100 nt downstream of the start codon. Further, siRNAs with lower G/C content (35-55%) may be more active than those with G/C content higher than 55%. Thus in one embodiment, the invention includes nucleic acid molecules having 35-55% G/C content. In addition, the strands of the siRNA can be paired in such a way as to have a 3′ overhang of 1 to 4, e.g., 2, nucleotides. Thus in another embodiment, the nucleic acid molecules can have a 3′ overhang of 2 nucleotides, such as TT. The overhanging nucleotides can be either RNA or DNA.
2. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available on the world wide web at ncbi.nlm.nih.gov/BLAST.
3. Select one or more sequences that meet your criteria for evaluation.

Further general information about the design and use of siRNA can be found in “The siRNA User Guide,” available on the world wide web at mpibpc.gwdg.de/abteilungen/1100/105/sirna.html.

Negative control siRNAs should have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

siRNA Delivery for Longer-Term Expression

Synthetic siRNAs can be delivered into cells by cationic liposome transfection and electroporation. These exogenous siRNA show short term, transient persistence of the silencing effect (4˜5 days). Several strategies for expressing siRNA duplexes within cells from recombinant DNA constructs allow longer-term target gene suppression in cells, including mammalian Pol III promoter systems (e.g., HI or U6/snRNA promoter systems (Tuschl (2002), supra) capable of expressing functional double-stranded siRNAs; (Bagella et al., J. Cell. Physiol. 177:206-213 (1998); Lee et al. (2002), supra; Miyagishi et al. Nature Biotechnol. 20(5):497-500 (2002); Paul et al. (2002), supra; Yu et al. (2002), supra; Sui et al. (2002), supra). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by HI or U6 snRNA promoter and expressed in cells, can inhibit target gene expression (Bagella et al. (1998), supra; Lee et al. (2002), supra; Miyagishi et al. (2002), supra; Paul et al. (2002), supra; Yu et al. (2002), supra; Sui et al. (2002) supra). Constructs containing siRNA sequence under the control of T7 promoter also make functional siRNs when cotransfected into the cells with a vector expression T7 RNA polymerase (Jacque (2002), Nature 418(6896):435-8).

Animal cells natively express a range of noncoding RNAs of approximately 22 nucleotides termed micro RNA (mRNAs) and can regulate gene expression at the post transcriptional or translational level during animal development. One common feature of mRNAs is that they are all excised from an approximately 70 nucleotide precursor RNA stem-loop, probably by Dicer, an RNase 111-type enzyme, or a homolog thereof. By substituting the stem sequences of the mRNA precursor with mRNA sequence complementary to the target mRNA, a vector construct that expresses the novel mRNA can be used to produce siRNAs to initiate RNAi against specific mRNA targets in mammalian cells (Zeng (2002), supra). When expressed by DNA vectors containing polymerase III promoters, micro-RNA designed hairpins can silence gene expression (McManus (2002), supra). Viral-mediated delivery mechanisms can also be used to induce specific silencing of targeted genes through expression of siRNA, for example, by generating recombinant adenoviruses harboring siRNA under RNA Pol II promoter transcription control (Xia et al. Nature Biotechnol. 20(10):1006-1010 (2002). Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. Id. In an animal model, whole-embryo electroporation can efficiently deliver synthetic siRNA into post-implantation mouse embryos (Calegari et al., Proc. Natl. Acad. Sci. USA 99(22):14236-40 (2002)). In adult mice, efficient delivery of siRNA can be accomplished by “high-pressure” delivery technique, a rapid injection (within 5 seconds) of a large volume of siRNA containing solution into animal via the tail vein (Liu (1999), supra; McCaffrey, Nature 48(6893):38-9 (2002); Lewis, Nature Genetics 32:107-108 (2002)). Nanoparticles and liposomes can also be used to deliver siRNA into animals.

Uses of Engineered RNA Precursors to Induce RNAI

Engineered RNA precursors, introduced into cells or whole organisms, can lead to the production of a desired siRNA molecule. Such an siRNA molecule will then associate with endogenous protein components of the RNAi pathway to bind to and target a specific mRNA sequence for cleavage and destruction. In this fashion, the mRNA to be targeted by the siRNA generated from the engineered RNA precursor will be depleted from the cell or organism, leading to a decrease in the concentration of the protein encoded by that mRNA in the cell or organism.

Modified Acheron Nucleic Acid Molecules

The nucleic acid compositions of the invention can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al., Drug Deliv. Rev.:47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

The nucleic acid molecules of the present invention can also be labeled using any method known in the art; for instance, the nucleic acid compositions can be labeled with a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include quantum dots, umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ³²P or 3H, inter alia. The labeling can be carried out using methods known in the art, including commercially available kits, e.g., the SILENCER™ siRNA labeling kit (Ambion).

Isolated Acheron Polypeptides

In another aspect, the invention features isolated Acheron polypeptides or fragments thereof for use as immunogens or antigens to raise or test (or more generally to bind) anti-Acheron antibodies. Acheron protein can be isolated from cells or tissue sources using standard protein purification techniques. Acheron protein or fragments thereof can be produced by recombinant DNA techniques or synthesized chemically.

Polypeptides of the invention include those that arise as a result of the existence of multiple genes, alternative transcription events, alternative RNA splicing events, and alternative translational and post-translational events. The polypeptides can be expressed in systems, e.g., cultured cells, that result in substantially the same post-translational modifications present when expressed the polypeptide is expressed in a native cell, or in systems that result in the alteration or omission of post-translational modifications, e.g., glycosylation or cleavage, present when expressed in a native cell.

In one embodiment, an Acheron polypeptide has one or more of the following characteristics:
(i) it has the ability to modulate apoptosis or differentiation;
(ii) it has a molecular weight, e.g., a deduced molecular weight, typically ignoring any contribution of post translational modifications, amino acid composition or other physical characteristic, of SEQ ID NO:4;
(iii) it has an overall sequence similarity of at least 60, 70, 80, 90, or 95%, with a polypeptide of SEQ ID NO:4; and/or
(iv) it comprises one or more of the following: a region of SEQ ID NO:4 corresponding to one or more of the following: residues 1-33; residues 34-491;
(v) it comprises one or more of the Acheron functional domains.

In one embodiment the Acheron protein, or fragment thereof, differs from the corresponding sequence in SEQ ID NO:4. In one embodiment it differs by at least one but by fewer than 15, 10, or 5 amino acid residues. (If this comparison requires alignment the sequences should be aligned for maximum homology. “Looped” out sequences from deletions or insertions, or mismatches, are considered differences.) The differences are, typically, differences or changes at a non essential residue or a conservative substitution. In one embodiment the differences are not in any of: a region of SEQ ID NO:4 corresponding to one or more of the following: residues 1-33; residues 34-491; all or part of one or more of the Acheron functional domains. In another embodiment one or more differences are in one or more of: a region of SEQ ID NO:4 corresponding to one or more of the following: residues 1-33; residues 34-491; all or part of one or more of the Acheron functional domains. In one embodiment, the Acheron protein differs from the sequence in SEQ ID NO:4 at least by lacking the first (N-terminal) 33 amino acids, e.g. an N-terminally truncated form of Acheron.

Other embodiments include a protein that contain one or more changes in amino acid sequence, e.g., a change in an amino acid residue that is not essential for activity. Such Acheron proteins differ in amino acid sequence from SEQ ID NO:4, yet retain biological activity.

In one embodiment, the Acheron protein includes an amino acid sequence that is at least about 95%, 96%, 97%, 98%, 99% or more identical to SEQ ID NO:4.

Acheron Chimeric or Fusion Proteins

In another aspect, the invention provides Acheron chimeric or fusion proteins. As used herein, an Acheron “chimeric protein” or “fusion protein” includes an Acheron polypeptide linked to a non-Acheron polypeptide. A “non-Acheron polypeptide” refers to a polypeptide having an amino acid sequence corresponding to a protein that is not substantially homologous to the Acheron protein, e.g., a protein that is different from the Acheron protein and that is derived from the same or a different organism. The Acheron polypeptide of the fusion protein can correspond to all or a portion e.g., a fragment described herein of an Acheron amino acid sequence. In one embodiment, an Acheron fusion protein includes at least one (or two) biologically active portion of an Acheron protein. The non-Acheron polypeptide can be fused to the N-terminus or C-terminus of the Acheron polypeptide, but is typically fused to the C-terminus.

The fusion protein can include a moiety that has a high affinity for a ligand. For example, the fusion protein can be a GST-Acheron fusion protein in which the Acheron sequences are fused to the C-terminus of the GST sequences. As another example, the fusion protein can be a FLAG®-Acheron fusion protein in which one or more FLAG® sequences (Asp-Tyr-Lys-Asp-Asp-Asp-Asp-Lys; SEQ ID NO:8) are fused to the N-terminus of Acheron. Such fusion proteins can facilitate the purification and/or detection of recombinant Acheron. Alternatively, the fusion protein can be an Acheron protein containing a heterologous signal sequence at its N-terminus. In certain host cells (e.g., mammalian host cells), expression and/or secretion of Acheron can be increased through use of a heterologous signal sequence.

Fusion proteins can include all or a part of a serum protein, e.g., an IgG constant region, or human serum albumin.

The Acheron fusion proteins of the invention can be incorporated into pharmaceutical compositions and administered to a subject in vivo. The Acheron fusion proteins can be used to affect the bioavailability of an Acheron substrate. Acheron fusion proteins may be useful therapeutically for the treatment of disorders caused by, for example, (i) aberrant modification or mutation of a gene encoding an Acheron protein; (ii) mis-regulation of the Acheron gene; and (iii) aberrant post-translational modification of an Acheron protein.

Moreover, the Acheron-fusion proteins of the invention can be used as immunogens to produce anti-Acheron antibodies in a subject, to purify Acheron ligands and in screening assays to identify molecules that inhibit the interaction of Acheron with an Acheron substrate.

Expression vectors are known and commercially available that include nucleic acid sequences that encode a fusion moiety (e.g., a GST polypeptide or FLAG peptide). An Acheron-encoding nucleic acid can be cloned into such an expression vector such that the fusion moiety is linked in-frame to the Acheron protein.

Variants of Acheron Proteins

In another aspect, the invention also features a variant of an Acheron polypeptide, e.g., a polypeptide that functions as an agonist (mimetics) or as an antagonist. Variants of the Acheron proteins can be generated by mutagenesis, e.g., discrete point mutation, the insertion or deletion of sequences or the truncation of an Acheron protein. An agonist variant of the Acheron protein can retain substantially the same, or a subset, of the biological activities of the naturally occurring form of an Acheron protein. An antagonist variant of an Acheron protein can inhibit one or more of the activities of the naturally occurring form of the Acheron protein by, for example, competitively modulating an Acheron-mediated activity of an Acheron protein, e.g., by acting as a dominant negative. Thus, specific biological effects can be elicited by treatment with a variant of limited function.

Variants of an Acheron protein can be identified by screening combinatorial libraries of mutants, e.g., truncation mutants, of an Acheron protein for agonist or antagonist activity.

Libraries of fragments e.g., N terminal, C terminal, or internal fragments, of an Acheron protein coding sequence can be used to generate a variegated population of fragments for screening and subsequent selection of variants of an Acheron protein.

Variants in which one or more cysteine residues are added or deleted or in which a residue that is glycosylated is added or deleted can also be used.

Methods for screening gene products of combinatorial libraries made by point mutations or truncation, and for screening cDNA libraries for gene products having a selected property are known in the art. Recursive ensemble mutagenesis (REM), a new technique that enhances the frequency of functional mutants in the libraries, can be used in combination with the screening assays to identify Acheron variants (Arkin and Yourvan, Proc. Natl. Acad. Sci. USA 89:7811-7815 (1992); Delgrave et al., Protein Engineering 6:327-331 (1993).

Cell based assays can be exploited to analyze a variegated Acheron library. For example, a library of expression vectors can be transfected into a cell line, e.g., a cell line, which ordinarily responds to Acheron in a substrate-dependent manner. The transfected cells are then contacted with Acheron and the effect of the expression of the mutant on signaling by the Acheron substrate can be detected, e.g., by measuring apoptosis. Plasmid DNA can then be recovered from the cells that score for inhibition, or alternatively, potentiation of signaling by the Acheron substrate, and the individual clones further characterized.

In another aspect, the invention features a method of making an Acheron polypeptide, e.g., a peptide having a non-wild type activity, e.g., an antagonist, agonist, or super agonist of a naturally occurring Acheron polypeptide. The method includes altering the sequence of an Acheron polypeptide, e.g., by substitution or deletion of one or more residues of a non-conserved region, a domain or residue disclosed herein, and testing the altered polypeptide for the desired activity. In some embodiments, the domain is a region of SEQ ID NO:4 corresponding to one or more of the following: residues 1-33; residues 34-491; or all or part of one or more of the Acheron functional domains.

In some embodiments, the antagonist variant is a dominant negative form of Acheron, e.g., an N-terminally truncated form of Acheron, e.g., a variant lacking the first 33 amino acids of SEQ ID NO:4. In some embodiments, the variant comprises a region of SEQ ID NO:4 corresponding to one or more of the following: residues 1-33; residues 34-491; or all or part of an Acheron functional domain, as described herein.

In another aspect, the invention features a method of making a fragment or analog of an Acheron polypeptide that has at least one biological activity of a naturally occurring Acheron polypeptide. The method includes: altering the sequence, e.g., by substitution or deletion of one or more residues, of an Acheron polypeptide, e.g., altering the sequence of a non-conserved region, or a domain or residue described herein, and testing the altered polypeptide for the desired activity. In some embodiments, the altered domain is a region of SEQ ID NO:4 corresponding to one or more of the following: residues 1-33; residues 34-491; or all or part of one or more Acheron functional domain, as described herein.

Anti-Acheron Antibodies

In another aspect, the invention includes anti-Acheron antibodies. The term “antibody” as used herein refers to an immunoglobulin molecule or immunologically active portion thereof, i.e., an antigen-binding portion. Examples of immunologically active portions of immunoglobulin molecules include Fv, F(ab), and F(ab′)₂ fragments that can be generated by treating the antibody with an enzyme such as pepsin.

The antibody can be a polyclonal, monoclonal, recombinant, e.g., a chimeric or humanized, fully human, non-human, e.g., murine, or single chain antibody. In one embodiment it has effector function and can fix complement. The antibody can be coupled to a toxin or imaging agent.

Methods for making monoclonal antibodies are known in the art. Basically, the process involves obtaining antibody-secreting immune cells (lymphocytes) from the spleen of a mammal (e.g., mouse) that has been previously immunized with the antigen of interest (e.g., Acheron) either in vivo or in vitro. The antibody-secreting lymphocytes are then fused with myeloma cells or transformed cells that are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference.

Mammalian lymphocytes are immunized by in vivo immunization of the animal (e.g., a mouse) with the protein or polypeptide of the invention, e.g., Acheron. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by known techniques, for example, using polyethylene glycol (“PEG”) or other fusing agents (See Milstein and Kohler, Eur. J. Immunol. 6:511 (1976), which is hereby incorporated by reference). This immortal cell line, which is preferably murine, but can also be derived from cells of other mammalian species, including but not limited to rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also known. Typically, such antibodies can be raised by administering the protein or polypeptide of the present invention subcutaneously to New Zealand white rabbits that have first been bled to obtain pre-immune serum. The antigens can be injected at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthanized, e.g., with pentobarbital 150 mg/Kg IV. This and other procedures for raising polyclonal antibodies are disclosed in E. Harlow, et. al., editors, Antibodies: A Laboratory Manual (1988).

In addition to utilizing whole antibodies, the invention encompasses the use of binding portions of such antibodies. Such binding portions include Fab fragments, F(ab′)₂ fragments, and Fv fragments. These antibody fragments can be made by conventional procedures, such as proteolytic fragmentation procedures, as described in J. Goding, Monoclonal Antibodies: Principles and Practice, pp. 98-118 (N.Y. Academic Press 1983).

A full-length Acheron protein or antigenic peptide fragment of Acheron can be used as an immunogen or can be used to identify anti-Acheron antibodies made with other immunogens, e.g., cells, membrane preparations, and the like. The antigenic peptide of Acheron should include at least 8 amino acid residues of the amino acid sequence shown in SEQ ID NO:4 and encompass an epitope of Acheron. Typically, the antigenic peptide includes at least 10, 15, 20, or 30 amino acid residues. In some embodiments, the antigenic peptide is a region of SEQ ID NO:4 corresponding to one or more of the following: residues 1-33; residues 34-491; or all or part of an Acheron functional domain, as described herein.

Fragments of Acheron can be used to make antibodies against regions of the Acheron protein, e.g., used as immunogens or used to characterize the specificity of an antibody. Antibodies reactive with, or specific for, any of these regions, or other regions or domains described herein are provided. Specific regions, such as hydrophobic regions, hydrophilic regions, or regions predicted to have high antigenicity can be identified using methods known in the art.

Epitopes encompassed by the antigenic peptide can include regions of Acheron located on the surface of the protein, e.g., hydrophilic regions, as well as regions with high antigenicity. For example, an Emini surface probability analysis of the human Acheron protein sequence can be used to indicate the regions that have a particularly high probability of being localized to the surface of the Acheron protein and are thus likely to constitute surface residues useful for targeting antibody production.

In one embodiment the antibody binds an epitope on any domain or region on Acheron proteins described herein.

Chimeric, humanized, deimmunized and completely human antibodies as known in the art are desirable for applications that include repeated administration, e.g., therapeutic treatment (and some diagnostic applications) of human patients.

The anti-Acheron antibody can be a single chain antibody. A single-chain antibody (scFV) may be engineered (see, for example, Colcher, D. et al. (1999) Ann NY Acad Sci 880:263-80; and Reiter, Y. (1996) Clin Cancer Res 2:245-52). The single chain antibody can be dimerized or multimerized to generate multivalent antibodies having specificities for different epitopes of the same target Acheron protein.

In one embodiment, the antibody has reduced or no ability to bind an Fc receptor. For example., it is an isotype or subtype, fragment or other mutant, which does not support binding to an Fc receptor, e.g., it has a mutated or deleted Fc receptor binding region.

An anti-Acheron antibody as described herein can be used to isolate Acheron by standard techniques, such as affinity chromatography or immunoprecipitation. Moreover, an anti-Acheron antibody can be used to detect Acheron protein (e.g., in a cellular lysate, cell supernatant, or tissue sample, e.g., a biopsy sample) in order to evaluate the abundance, pattern of expression, and subcellular localization of the protein. Anti-Acheron antibodies can be used diagnostically to monitor protein levels in tissue, or subcellular localization, as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, P-galactosidase, or acetylcholinesterase; examples of suitable prosthetic group complexes include streptavidin/biotin and avidin/biotin; examples of suitable fluorescent materials include quantum dots, umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H, inter alia.

Recombinant Expression Vectors, Host Cells and Genetically Engineered Cells

In another aspect, the invention includes vectors, such as expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include an Acheron nucleic acid in a form suitable for expression of the nucleic acid in a host cell. Typically, the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., Acheron proteins, mutant forms of Acheron proteins, fusion proteins, and the like).

The recombinant expression vectors of the invention can be designed for expression of Acheron proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in Acheron activity assays, (e.g., direct assays or competitive assays described in detail below), or to generate antibodies specific for Acheron proteins. In one embodiment, a fusion protein expressed in a retroviral expression vector of the present invention can be used to infect bone marrow cells that are subsequently transplanted into irradiated recipients. The pathology of the subject recipient is then examined after sufficient time has passed (e.g., six weeks).

To maximize recombinant protein expression in E. coli, one can express the protein in a host bacteria that has an impaired capacity to proteolytically cleave the recombinant protein (Gottesman, S., (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The Acheron expression vector can be e.g., a yeast expression vector, a vector for expression in insect cells, e.g., a baculovirus expression vector, or a vector suitable for expression in mammalian cells.

When used in mammalian cells, the expression vector's control functions can be provided by viral regulatory elements. For example, commonly used promoters are derived from polyoma, Adenovirus 2, cytomegalovirus and Simian Virus 40.

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen that direct the constitutive, tissue specific or cell type specific expression of antisense RNA in a variety of cell types. The antisense expression vector can be in the form of a recombinant plasmid, phagemid or attenuated virus. For a discussion of the regulation of gene expression using antisense genes see Weintraub, H. et al., Reviews—Trends in Genetics 1:1 (1986).

Another aspect of the invention provides a host cell that includes a nucleic acid molecule described herein, e.g., an Acheron nucleic acid molecule within a recombinant expression vector or an Acheron nucleic acid molecule containing sequences that allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an Acheron protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) an Acheron protein. Accordingly, the invention further provides methods for producing an Acheron protein using the host cells of the invention. In one embodiment, the method includes culturing the host cell of the invention (into which a recombinant expression vector encoding an Acheron protein has been introduced) in a suitable medium such that an Acheron protein is produced. In another embodiment, the method further includes isolating an Acheron protein from the medium or the host cell.

In another aspect, the invention features a cell or purified preparation of cells that include an Acheron transgene, or which otherwise misexpress Acheron. The cell preparation can consist of human or non human cells, e.g., rodent cells, e.g., mouse or rat cells, rabbit cells, Chinese hamster ovary (CHO) cells, or pig cells. In some embodiments, the cell or cells include an Acheron transgene, e.g., a heterologous form of Acheron, e.g., a gene derived from humans in the case of a non-human cell. The Acheron transgene can be misexpressed, e.g., overexpressed, underexpressed, or mislocalized. In other embodiments, the cell or cells include a gene that misexpresses an endogenous Acheron, e.g., a gene the expression of which is disrupted, e.g., a knockout. Such cells can serve as a model for studying disorders that are related to mutated or misexpressed Acheron alleles or for use in drug screening.

In another aspect, the invention features a cell, e.g., a mammalian cell, e.g., a myoblast, neural stem cell, or hematopoietic stem cell, transformed with nucleic acid that encodes an Acheron polypeptide.

Also provided are cells, e.g., human cells, e.g., human neural, hematopoietic, or myoblast cells, in which an endogenous Acheron is under the control of a regulatory sequence that does not normally control the expression of the endogenous Acheron gene. The expression characteristics of an endogenous gene within a cell, e.g., a cell line or microorganism, can be modified by inserting a heterologous DNA regulatory element into the genome of the cell such that the inserted regulatory element is operably linked to the endogenous Acheron gene. For example, an endogenous Acheron gene that is “transcriptionally silent,” e.g., not normally expressed, or expressed only at very low levels, may be activated by inserting a regulatory element that is capable of promoting the expression of a normally expressed gene product in that cell. Techniques such as targeted homologous recombination, can be used to insert the heterologous DNA as described in, e.g., Chappel, U.S. Pat. No. 5,272,071; WO 91/06667, published in May 16, 1991.

In another aspect, the invention provides isolated engineered Acheron host cells suitable for transplantation into a subject, e.g., a cell for use in cell-transplantation based genetic therapies or other transplant therapies where increased survival of transplanted cells is desirable. Engineered cells are cells in which a change has occurred due to human intervention which includes both permanent changes (e.g., cells stably expressing an Acheron transgene, or Acheron knock-out cells), and transient changes (e.g., cells treated with an Acheron inhibitor, e.g., Acheron antisense, antibody, siRNA, or dominant negative polypeptide). For example, such a cell could be an autologous or heterologous stem cell or a partially differentiated cell, including, but not limited to, neural progenitor cells and muscle progenitor cells (e.g., myoblasts). In some embodiments, the host cell will also express one or more additional ectopic genes, e.g., non-Acheron genes intended to enhance the survival of transplanted cells, or genes intended to treat a disease e.g., dystrophin or SOD-1. Such genes may include genes intended to correct a genetic defect, e.g., a mutation. In some embodiments, the host cells are autologous, e.g., taken from an intended transplant recipient. In some embodiments, the host cells misexpress Acheron, e.g., have increased or decreased Acheron activity. For example, cells with decreased Acheron activity, e.g., genetically engineered cells lacking all or part of the Acheron gene or expressing Acheron antisense or ds-siRNA or an Acheron dominant negative, are less likely to undergo apoptosis and thus have an enhanced chance of survival when transplanted into a recipient. In some embodiments, the cells have been treated with a transient inhibitor of Acheron expression or activity, e.g., an Acheron antisense, antibody, siRNA, or dominant negative Acheron polypeptide.

Transgenic Animals

The invention provides non-human transgenic animals. Such animals are useful for studying the function and/or activity of an Acheron protein and for identifying and/or evaluating modulators of Acheron activity. As used herein, a “transgenic animal” is a non-human animal, e.g., a mammal, typically a rodent such as a rat or mouse, in which one or more of the cells of the animal includes an Acheron transgene. Other examples of transgenic animals include non-human primates, sheep, dogs, cows, goats, chickens, amphibians, and the like. A transgene is exogenous DNA or a rearrangement, e.g., a deletion of endogenous chromosomal DNA, which can be integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous Acheron gene has been altered by, e.g., by homologous recombination between the endogenous gene and an exogenous DNA molecule introduced into a cell of the animal, e.g., an embryonic cell of the animal, prior to development of the animal.

Intronic sequences and polyadenylation signals can also be included in the transgene to increase the efficiency of expression of the transgene. A tissue-specific regulatory sequence(s) can be operably linked to a transgene of the invention to direct expression of an Acheron protein to particular cells. A transgenic founder animal can be identified based upon the presence of an Acheron transgene in its genome and/or expression of Acheron mRNA in tissues or cells of the animals. A transgenic founder animal can then be used to breed additional animals carrying the transgene. Moreover, transgenic animals carrying a transgene encoding an Acheron protein can further be bred to other transgenic animals carrying other transgenes.

Acheron proteins or polypeptides can be expressed in transgenic animals or plants, e.g., a nucleic acid encoding the protein or polypeptide can be introduced into the genome of an animal. In some embodiments the nucleic acid is placed under the control of a tissue specific promoter, e.g., a milk or egg specific promoter, and recovered from the milk or eggs produced by the animal. Suitable animals are mice, pigs, cows, goats, and sheep.

The invention also includes a population of cells from a transgenic animal.

Uses

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic), of cellular proliferative and/or differentiative disorders, and disorders associated with cellular degeneration, e.g., as described herein.

The isolated nucleic acid molecules of the invention can be used, for example, to express an Acheron protein (e.g., via a recombinant expression vector in a host cell in gene therapy applications), to detect an Acheron mRNA (e.g., in a biological sample) or a genetic alteration in an Acheron gene, and to modulate Acheron activity, as described further below. The Acheron proteins can be used to treat disorders characterized by insufficient or excessive production of an Acheron substrate or production of Acheron inhibitors. In addition, the Acheron proteins can be used to screen for naturally occurring Acheron substrates, to screen for drugs or compounds that modulate Acheron activity, as well as to treat disorders characterized by insufficient or excessive production of Acheron protein or production of Acheron protein forms that have decreased, aberrant, or unwanted activity compared to Acheron wild type protein (e.g., disorders associated with aberrant cell differentiation, proliferation, or degeneration). Such disorders include cellular proliferative and/or differentiative disorders, and disorders associated with cellular degeneration, e.g., as described herein. Moreover, the anti-Acheron antibodies of the invention can be used to detect and isolate Acheron proteins, regulate the bioavailability of Acheron proteins, and modulate Acheron activity.

A method of evaluating a compound for the ability to interact with, e.g., bind, a subject Acheron polypeptide is provided. The method includes contacting the compound with the subject Acheron polypeptide; and evaluating ability of the compound to interact with, e.g., to bind or form a complex with the subject Acheron polypeptide. This method can be performed in vitro, e.g., in a cell free system, or in vivo, e.g., in a two-hybrid interaction trap assay. This method can be used to identify naturally occurring molecules that interact with subject Acheron polypeptide. It can also be used to find natural or synthetic inhibitors of subject Acheron polypeptide. Screening methods are discussed in more detail below.

Methods for Identifying Modulators of Acheron

The invention provides methods (also referred to herein as “screening assays”) for identifying modulators, i.e., candidate or test compounds or agents (e.g., proteins, peptides, peptidomimetics, peptoids, small molecules or other drugs) that bind to Acheron proteins, have a stimulatory or inhibitory effect on, for example, Acheron expression or Acheron activity, or have a stimulatory or inhibitory effect on, for example, the expression or activity of an Acheron substrate. Compounds thus identified can be used to modulate the activity of target gene products (e.g., Acheron genes) in a therapeutic protocol, to elaborate the biological function of the target gene product, or to identify compounds that disrupt normal target gene interactions.

In one embodiment, the invention provides assays for screening candidate or test compounds that are substrates of an Acheron protein or polypeptide or a biologically active portion thereof. In another embodiment, the invention provides assays for screening candidate or test compounds that bind to or modulate the activity of an Acheron protein or polypeptide or a biologically active portion thereof.

The test compounds of the present invention can be obtained using any of the numerous approaches in combinatorial library methods known in the art, including: biological libraries; peptoid libraries (libraries of molecules having the functionalities of peptides, but with a novel, non-peptide backbone that are resistant to enzymatic degradation but that nevertheless remain bioactive; see, e.g., Zuckermann, R. N. et al., J. Med. Chem. 37:2678-85 (1994); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, K. S., Anticancer Drug Des. 12:145 (1997).

Examples of methods for the synthesis of molecular libraries can be found in the art, for example in DeWitt et al. Proc. Natl. Acad. Sci. U.S.A. 90:6909 (1993); Erb et al. Proc. Natl. Acad. Sci. USA 91:11422 (1994); Zuckermann et al. J. Med. Chem. 37:2678 (1994); Cho et al. Science 261:1303 (1993); Carrell et al. Angew. Chem. Int. Ed. Engl. 33:2059 (1994); Carell et al. Angew. Chem. Int. Ed. Engl. 33:2061 (1994); and Gallop et al. J. Med. Chem. 37:1233 (1994).

Libraries of compounds may be presented in solution (e.g., Houghten, Biotechniques 13:412-421(1992), or on beads (Lam, Nature 354:82-84 (1991), chips (Fodor, Nature 364:555-556 (1993), bacteria (Ladner, U.S. Pat. No. 5,223,409), spores (Ladner U.S. Pat. No. '409), plasmids (Cull et al. Proc Natl Acad Sci USA 89:1865-1869 (1992) or on phage (Scott and Smith, Science 249:386-390 (1990); Devlin, Science 249:404-406 (1990); Cwirla et al. Proc. Natl. Acad. Sci. 87:6378-6382 (1990); Felici, J. Mol. Biol. 222:301-310 (1991); Ladner supra.).

In one embodiment, an assay is a cell-based assay in which a cell that expresses an Acheron protein or biologically active portion thereof is contacted with a test compound, and the ability of the test compound to modulate Acheron activity is determined. Determining the ability of the test compound to modulate Acheron activity can be accomplished by monitoring, for example, apoptosis or cell differentiation. The cell, for example, can be of mammalian origin, e.g., mouse, rat, or human.

The ability of the test compound to modulate Acheron binding to a compound, e.g., an Acheron substrate or binding partner such as CASK-C or Ariadne, or to bind to Acheron can also be evaluated. This can be accomplished, for example, by coupling the compound, e.g., the substrate, with a radioisotope or enzymatic label such that binding of the compound, e.g., the substrate, to Acheron can be determined by detecting the labeled compound, e.g., substrate, in a complex. Alternatively, Acheron could be coupled with a radioisotope or enzymatic label to monitor the ability of a test compound to modulate Acheron binding to an Acheron substrate in a complex. For example, compounds (e.g., Acheron substrates) can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., an Acheron substrate) to interact with Acheron with or without the labeling of any of the interactants can be evaluated. For example, a microphysiometer can be used to detect the interaction of a compound with Acheron without the labeling of either the compound or the Acheron. McConnell, H. M. et al. Science 257:1906-1912 (1992). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and Acheron.

In yet another embodiment, a cell-free assay is provided in which an Acheron protein or biologically active portion thereof is contacted with a test compound and the ability of the test compound to bind to the Acheron protein or biologically active portion thereof is evaluated. Biologically active portions of the Acheron proteins to be used in assays of the present invention include fragments that participate in interactions with non-Acheron molecules, e.g., fragments with high surface probability scores.

Soluble and/or membrane-bound forms of isolated proteins (e.g., Acheron proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor.’ Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of the Acheron protein to bind to a target molecule can be accomplished using real-time Biomolecular Interaction Analysis (BIA) (see, e.g., Sjolander, S. and Urbaniczky, C. Anal. Chem. 63:2338-2345 (1991) and Szabo et al. Curr. Opin. Struct. Biol. 5:699-705 (1995). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the target gene product or the test substance is anchored onto a solid phase. The target gene product/test compound complexes anchored on the solid phase can be detected at the end of the reaction. For example, the target gene product can be anchored onto a solid surface, and the test compound, (which is not anchored), can be labeled, either directly or indirectly, with detectable labels discussed herein.

It may be desirable to immobilize either Acheron, an anti-Acheron antibody, or its target molecule to facilitate separation of complexed from uncomplexed forms of one or both of the proteins, as well as to accommodate automation of the assay. Binding of a test compound to an Acheron protein, or interaction of an Acheron protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, glutathione-S-transferase/Acheron fusion proteins or glutathione-S-transferase/target fusion proteins can be adsorbed onto glutathione Sepharose® beads (Sigma Chemical, St. Louis, Mo.) or glutathione derivatized microtiter plates, which are then combined with the test compound or the test compound and either the non-adsorbed target protein or Acheron protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of Acheron binding or activity determined using standard techniques.

Other techniques for immobilizing either an Acheron protein or a target molecule on matrices include using conjugation of biotin and streptavidin. Biotinylated Acheron protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical).

To conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies that are reactive with Acheron protein or target molecules but that do not interfere with binding of the Acheron protein to its target molecule. Such antibodies can be derivatized to the wells of the plate, and unbound target or Acheron protein trapped in the wells by antibody conjugation. Methods for detecting such complexes, in addition to those described above for the GST-immobilized complexes, include immunodetection of complexes using antibodies reactive with the Acheron protein or target molecule, as well as enzyme-linked assays that rely on detecting an enzymatic activity associated with the Acheron protein or target molecule.

Alternatively, cell free assays can be conducted in a liquid phase. In such an assay, the reaction products are separated from unreacted components, by any of a number of standard techniques, including but not limited to differential centrifugation (see, for example, Rivas, G., and Minton, A. P., Trends Biochem Sci 18:284-7 (1993); chromatography (gel filtration chromatography, ion-exchange chromatography); electrophoresis (see, e.g., Ausubel et al. eds. Current Protocols in Molecular Biology 1999, J. Wiley: New York.); and immunoprecipitation (see, for example, Ausubel et al., eds. (1999) supra). Such resins and chromatographic techniques are known to one skilled in the art (see, e.g., Heegaard, J Mol Recognit 11: 141-8 (1998); Hage, D. S., and Tweed, S. A. J Chromatogr B Biomed Sci Appl. 699:499-525 (1997). Further, fluorescence energy transfer may also be conveniently utilized, as described herein, to detect binding without further purification of the complex from solution.

In one embodiment, the assay includes contacting the Acheron protein or biologically active portion thereof with a known compound that binds Acheron to form an assay mixture, contacting the assay mixture with a test compound, and determining the ability of the test compound to interact with an Acheron protein, wherein determining the ability of the test compound to interact with an Acheron protein includes determining the ability of the test compound to preferentially bind to Acheron or biologically active portion thereof, or to modulate the activity of a target molecule, as compared to the known compound.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to, molecules such as antibodies, peptides, and small molecules. Typically, the target genes/products for use in this embodiment are the Acheron genes herein described. In an alternative embodiment, the invention provides methods for determining the ability of the test compound to modulate the activity of an Acheron protein through modulation of the activity of a downstream effector of an Acheron target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. To test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

To conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit the complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified. In yet another aspect, the Acheron proteins can be used as “bait proteins” in a two-hybrid assay or three-hybrid assay (see, e.g., U.S. Pat. No. 5,283,317; Zervos et al. Cell 72:223-232 (1993); Madura et al. J. Biol. Chem. 268:12046-12054 (1993); Bartel et al. Biotechniques 14:920-924 (1993); Iwabuchi et al. Oncogene 8:1693-1696 (1993); and Brent WO94/10300), to identify proteins that bind to or interact with Acheron and are involved in Acheron activity. Such Acheron-binding proteins can be activators or inhibitors of signals by the Acheron proteins or Acheron targets as, for example, downstream elements of an Acheron-mediated signaling pathway. Proteins identified in this manner include CASK-C and Ariadne.

The two-hybrid system is based on the modular nature of most transcription factors, which consist of separable DNA-binding and activation domains. Briefly, the assay utilizes two different DNA constructs. In one construct, the gene that codes for an Acheron protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. Alternatively, the Acheron protein can be the fused to the activator domain. If the “bait” and the “prey” proteins are able to interact, in vivo, forming an Acheron-dependent complex, the DNA-binding and activation domains of the transcription factor are brought into close proximity. This proximity allows transcription of a reporter gene (e.g., lacZ) that is operably linked to a transcriptional regulatory site responsive to the transcription factor. Expression of the reporter gene can be detected and cell colonies containing the functional transcription factor can be isolated and used to obtain the cloned gene, which encodes the protein that interacts with the Acheron protein.

In another embodiment, modulators of Acheron expression are identified. For example, a cell or cell free mixture is contacted with a candidate compound and the expression of Acheron mRNA or protein evaluated relative to the level of expression of Acheron mRNA or protein in the absence of the candidate compound. When expression of Acheron mRNA or protein is greater in the presence of the candidate compound than in its absence, the candidate compound is identified as a stimulator of Acheron mRNA or protein expression. Alternatively, when expression of Acheron mRNA or protein is less (statistically significantly less) in the presence of the candidate compound than in its absence, the candidate compound is identified as an inhibitor of Acheron mRNA or protein expression. The level of Acheron mRNA or protein expression can be determined by methods described herein for detecting Acheron mRNA or protein.

In another aspect, the invention pertains to a combination of two or more of the assays described herein. For example, a modulating agent can be identified using a cell-based or a cell free assay, and the ability of the agent to modulate the activity of an Acheron protein can be confirmed in vivo, e.g., in an animal such as an animal model for a disorder associated with aberrant cellular proliferation, differentiation, or degeneration.

This invention further pertains to novel agents identified by the above-described screening assays. Accordingly, it is within the scope of this invention to further use an agent identified as described herein (e.g., an Acheron modulating agent, an antisense Acheron nucleic acid molecule, an Acheron-specific antibody, or an Acheron-binding partner, e.g., CASK-C or Ariadne) in an appropriate animal model to determine the efficacy, toxicity, side effects, or mechanism of action, of treatment with such an agent. Furthermore, agents identified by the above-described screening assays, e.g., CASK-C and Ariadne, can be used for treatments as described herein.

Detection Assays

Portions or fragments of the nucleic acid sequences identified herein can be used as polynucleotide reagents. For example, these sequences can be used to (i) map their respective genes on a chromosome e.g., to locate gene regions associated with genetic disease or to associate Acheron with a disease; (ii) identify an individual from a minute biological sample (tissue typing); and (iii) aid in forensic identification of a biological sample. Methods for accomplishing these applications are known in the art.

Diagnostic and Prognostic Assays

The presence, level, subcellular localization or absence of Acheron protein or nucleic acid in a biological sample can be evaluated by obtaining a biological sample from a test subject and contacting the biological sample with a compound or an agent capable of detecting Acheron protein or nucleic acid (e.g., mRNA, genomic DNA) that encodes Acheron protein. The term “biological sample” includes tissues, cells, and biological fluids isolated from a subject. Typical biological samples include serum and tumor biopsy tissue. The level of expression of the Acheron gene can be measured in a number of ways, including, but not limited to, measuring the mRNA encoded by the Acheron genes; measuring the amount of protein encoded by the Acheron genes; or measuring the activity of the protein encoded by the Acheron genes. The subcellular localization of the Acheron protein can be measured by methods known in the art, including immunohistochemistry, e.g., using known pathology methods and the anti-Acheron antibodies described herein.

The level of mRNA corresponding to the Acheron gene in a cell can be determined both by in situ and by in vitro formats.

The isolated mRNA can be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe can be, for example, a full-length Acheron nucleic acid, such as the nucleic acid of SEQ ID NO:4, or a portion thereof, such as an oligonucleotide of at least 7, 15, 30, 50, 100, 250 or 500 nucleotides in length and sufficient to specifically hybridize under stringent conditions to Acheron mRNA or genomic DNA. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array. A skilled artisan can adapt known mRNA detection methods for use in detecting the level of mRNA encoded by Acheron genes.

The level of mRNA in a sample that is encoded by an Acheron gene can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self-sustained sequence replication (Guatelli et al., (1990) Proc. Natl. Acad. Sci. USA 87:1874-1878), transcriptional amplification system (Kwoh et al., (1989), Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033), or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the Acheron gene being analyzed.

In another embodiment, the methods further contacting a control sample with a compound or agent capable of detecting Acheron m RNA, or genomic DNA, and comparing the presence of Acheron mRNA or genomic DNA in the control sample with the presence of Acheron mRNA or genomic DNA in the test sample.

A variety of methods can be used to determine the level of protein encoded by an Acheron gene. In general, these methods include contacting an agent that selectively binds to the protein, such as an antibody with a sample, to evaluate the level of protein in the sample. In one embodiment, the antibody bears a detectable label. Antibodies can be polyclonal or monoclonal. An intact antibody, or a fragment thereof (e.g., Fv, Fab, or F(ab′)₂) can be used.

The detection methods can be used to detect Acheron protein in a biological sample in vitro as well as in vivo. In vitro techniques for detection of Acheron protein include enzyme linked immunosorbent assays (ELISAs), immunoprecipitations, immunofluorescence, enzyme immunoassay (EIA), radioimmunoassay (RIA), and Western blot analysis. In vivo techniques for detection of Acheron protein include introducing into a subject a labeled anti-Acheron antibody. For example, the antibody can be labeled with a marker, e.g., a radioactive marker, whose presence and location in a subject can be detected by standard imaging techniques.

In another embodiment, the methods further include contacting the control sample with a compound or agent capable of detecting Acheron protein, and comparing the presence of Acheron protein in the control sample with the presence of Acheron protein in the test sample.

The invention also includes kits for detecting the presence of Acheron in a biological sample. For example, the kit can include a compound or agent capable of detecting Acheron protein or mRNA in a biological sample and a standard. The compound or agent can be packaged in a suitable container. The kit can further comprise instructions for using the kit to detect Acheron protein or nucleic acid.

For antibody-based kits, the kit can include (1) a first antibody (e.g., attached to a solid support) that binds to a polypeptide corresponding to a marker of the invention, and, optionally, (2) a second, different antibody that binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein stabilizing agent. The kit can also includes components necessary for detecting the detectable agent (e.g., an enzyme or a substrate). The kit can also contain a control sample or a series of control samples that can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted Acheron expression or activity. As used herein, the term “unwanted” includes an undesirable phenomenon involved in a biological response such as pain or deregulated cell proliferation.

In one embodiment, a disease or disorder associated with aberrant or unwanted Acheron expression or activity, e.g., cellular proliferative and/or differentiative disorders, and disorders associated with cellular degeneration, e.g., as described herein, is identified. A test sample is obtained from a subject and Acheron protein or nucleic acid (e.g., mRNA or genomic DNA) is evaluated, wherein the level, e.g., the presence or absence, of Acheron protein or nucleic acid, or the subcellular localization of Acheron protein, is diagnostic for a subject having, or at risk of developing, a disease or disorder associated with aberrant or unwanted Acheron expression or activity.

For example, rhabdomyosarcoma-derived cell lines with Acheron localized to the nucleus are more aggressive and have a higher metastatic potential than cell lines lacking Acheron in the nucleus. Thus, the detection of tumor cells that have Acheron localized to the nucleus would indicate a tumor that has a high probability of metastasizing. Thus, in one embodiment, the presence, level, absence, or subcellular localization of Acheron protein indicates the grade of a tumor, e.g., whether the tumor is, or is likely to become, metastatic.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted Acheron expression or activity. For example, such methods can be used to determine whether a subject can be effectively treated with an agent for a disorder associated with aberrant Acheron activity or expression, e.g., a disorder associated with aberrant cellular proliferation, differentiation, or degeneration.

The methods of the invention can also be used to detect genetic alterations in an Acheron gene, thereby determining if a subject with the altered gene is at risk for a disorder characterized by misregulation in Acheron protein activity or nucleic acid expression, such as a disorder associated with aberrant cellular proliferation, differentiation, or degeneration. In some embodiments, the methods include detecting, in a sample from the subject, the presence or absence of an alteration characterized by at least one of an alteration affecting the integrity of a gene encoding an Acheron-protein, e.g., the mis-expression of the Acheron gene. For example, such alterations or mis-expression can be detected by ascertaining the existence of at least one of 1) a deletion of one or more nucleotides from an Acheron gene; 2) an addition of one or more nucleotides to an Acheron gene; 3) a substitution of one or more nucleotides of an Acheron gene, 4) a chromosomal rearrangement of an Acheron gene; 5) an alteration in the level of a messenger RNA transcript of an Acheron gene, 6) aberrant modification of an Acheron gene, such as of the methylation pattern of the genomic DNA, 7) the presence of a non-wild type splicing pattern of a messenger RNA transcript of an Acheron gene, 8) a non-wild type level of an Acheron protein, 9) allelic loss of an Acheron gene, 10) alterations in subcellular localization or levels of Acheron protein, and 11) inappropriate post-translational modification of an Acheron-protein.

An alteration can be detected without a probe/primer in a polymerase chain reaction, such as anchor PCR or RACE PCR, or, alternatively, in a ligation chain reaction (LCR), the latter of which can be particularly useful for detecting point mutations in the Acheron-gene. This method can include the steps of collecting a sample of cells from a subject, isolating nucleic acid (e.g., genomic, mRNA or both) from the sample, contacting the nucleic acid sample with one or more primers that specifically hybridize to an Acheron gene under conditions such that hybridization and amplification of the Acheron gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein. Alternatively, other amplification methods described herein or known in the art can be used.

In another embodiment, mutations in an Acheron gene from a sample cell can be identified by detecting alterations in restriction enzyme cleavage patterns. For example, sample and control DNA is isolated, amplified (optionally), digested with one or more restriction endonucleases, and fragment length sizes are determined, e.g., by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in Acheron can be identified by hybridizing a sample and control nucleic acids, e.g., DNA or RNA, two dimensional arrays, e.g., chip based arrays. Such arrays include a plurality of addresses, each of which is positionally distinguishable from the other. A different probe is located at each address of the plurality. The arrays can have a high density of addresses, e.g., can contain hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in Acheron can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations, and is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the Acheron gene and detect mutations by comparing the sequence of the sample Acheron with the corresponding wild-type (control) sequence. Automated sequencing procedures can be utilized when performing the diagnostic assays ((1995) Biotechniques 19:448), including sequencing by mass spectrometry.

Other methods for detecting mutations in the Acheron gene include methods in which protection from cleavage agents is used to detect mismatched bases in RNA/RNA or RNA/DNA heteroduplexes (Myers et al. (1985) Science 230:1242; Cotton et al. (1988) Proc. Natl. Acad Sci USA 85:4397; Saleeba et al. (1992) Methods Enzymol. 217:286-295).

In still another embodiment, the mismatch cleavage reaction employs one or more proteins that recognize mismatched base pairs in double-stranded DNA (so called “DNA mismatch repair” enzymes) in defined systems for detecting and mapping point mutations in Acheron cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662; U.S. Pat. No. 5,459,039).

In other embodiments, alterations in electrophoretic mobility will be used to identify mutations in Acheron genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control Acheron nucleic acids can be denatured and allowed to renature. The secondary structure of single-stranded nucleic acids varies according to sequence, the resulting alteration in electrophoretic mobility enables the detection of even a single base change. The DNA fragments can be labeled or detected with labeled probes. The sensitivity of the assay can be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension (Saiki et al. (1986) Nature 324:163); Saiki et al. (1989) Proc. Natl. Acad. Sci USA 86:6230).

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another embodiment, changes in protein levels or subcellular localization are detected, e.g., using a detectable agent that binds specifically to Acheron. Such agents can include anti-Acheron antibodies as described herein. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include quantum dots, umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H, inter alia.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an Acheron gene.

Pharmaceutical Compositions

The new nucleic acid molecules, polypeptides, and fragments thereof described herein, as well as anti-Acheron antibodies (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents can be included, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, possible methods of preparation include vacuum drying and freeze-drying that yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds can lie within a range of circulating concentrations that include the ED50 with little or no toxicity. The dosage may vary within this range depending upon the dosage form employed and the route of administration utilized. For any compound used in the methods described herein, the therapeutically effective dose can be estimated initially from cell culture assays. A dose may be tested in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, about 0.01 to 25 mg/kg body weight, about 0.1 to 20 mg/kg body weight, or about 1 to 10 mg/kg, 2 to 9 mg/kg, 3 to 8 mg/kg, 4 to 7 mg/kg, or 5 to 6 mg/kg body weight. The protein or polypeptide can be administered one or several times per day, every other day, or once a week for between about 1 to 10 weeks, about 2 to 8 weeks, about 3 to 7 weeks, or about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or can include a series of treatments.

For antibodies, the dosage can be about 0.1 to 100 mg/kg of body weight (generally 10 mg/kg to 20 mg/kg). If the antibody is to act in the brain, a dosage of 50 mg/kg to 100 mg/kg may be appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The present invention encompasses agents that modulate expression or activity. An agent may, for example, be a small molecule. For example, such small molecules include, but are not limited to, peptides, peptidomimetics (e.g., peptoids), amino acids, amino acid analogs, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram to about 500 milligrams per kilogram, about 100 micrograms to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

An antibody can be conjugated to a second antibody to form an antibody heteroconjugate as described by Segal in U.S. Pat. No. 4,676,980.

The Acheron nucleic acid molecules described herein can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Methods of Treating Disorders Associated with Aberrant Cellular Differentiation, Proliferation, or Degeneration

The present invention provides for both prophylactic and therapeutic methods of treating a subject at risk of (or susceptible to) a disorder or having a disorder associated with aberrant cellular differentiation, proliferation, or degeneration.

Cellular Proliferative and/or Differentiative Disorders

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer,” “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal, but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas that include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative and/or differentiative disorders include malignant and non-malignant muscle neoplastic disorders. As used herein, the term “muscle neoplastic disorders” includes diseases involving hyperplastic/neoplastic cells of muscle origin, e.g., arising from myoblasts. Such diseases include rhabdomyoma, leiomyoma, rhabdomyosarcoma, and leiomyosarcoma. Further examples of proliferative and/or differentiative disorders include tumors of neural origin, e.g., tumors originating or located in the central and/or peripheral nervous system, e.g., neuroblastoma, retinoblastoma, intracranial germinoma germ cell tumors, pediatric brain stem glioma, neuroblastoma, intrinsic pontine glioma, retinoblastoma, medulloblastoma, astrocytoma, acoustic neuroma, glioblastoma, meningioma, and oligodendroglioma. Since Acheron is expressed in oligodendrocytes, it may be an especially useful target in the treatment of various glioblastomas.

Human Acheron expression-positive staining is observed in the cytoplasm of the ganglion cells of the ganglion cell layer, the nuclei of a subset of neurons of the inner and outer nuclear layers and the outer segment of the cones. In the human retina, Acheron expression is observed in the cytoplasm of the ganglion cells of the ganglion cell layer, the nuclei of a subset of neurons of the inner and outer nuclear layers and the outer segment of the cones. Ganglionic cells of the submucosal plexus of Meissner display strong cytoplasmic human Acheron staining. Similar staining was observed in the ganglion cells of the myenteric plexus of Auerbach. Positive cytoplasmic staining was also present in the endothelial and smooth muscle cells of the vessels. Thus, inhibition of Acheron activity would be useful in the treatment of retinopathies, e.g., diabetic retinopathy, retinopathy of prematurity, macular degeneration and free radical-induced retinopathy, e.g., to inhibit the apoptotic loss of cells, e.g., cone cells.

Rhabdomyosarcoma (RMS) is the most common childhood soft tissue malignancy, accounting for 4-8% of all pediatric tumors. There are three major histological types: alveolar (15% of cases), which has an aggressive clinical course and poor prognosis; embryonal (85% of cases), which is less aggressive with better prognosis than the alveolar form, and pleomorphic, which is very rare. The alveolar type is characterized by the presence either of a t(2; 13) chromosomal translocation in about 68% of the cases or a t(1;13) in about 14%. Acheron is expressed in a number of RMS cell lines, thus, RMS can be treated by increasing Acheron activity, e.g., by a method described herein, such as introducing an Acheron nucleic acid, polypeptide, or functional fragment thereof, to the cell.

Other examples of proliferative and/or differentiative disorders include skin disorders. The skin disorder may involve the aberrant activity of a cell or a group of cells or layers in the dermal, epidermal, or hypodermal layer, or an abnormality in the dermal-epidermal junction. For example, the skin disorder may involve aberrant activity of keratinocytes (e.g., hyperproliferative basal and immediately suprabasal keratinocytes), melanocytes, Langerhans cells, Merkel cells, immune cell, and other cells found in one or more of the epidermal layers, e.g., the stratum basale (stratum germinativum), stratum spinosum, stratum granulosum, stratum lucidum or stratum corneum. In other embodiments, the disorder may involve aberrant activity of a dermal cell, e.g., a dermal endothelial, fibroblast, immune cell (e.g., mast cell or macrophage) found in a dermal layer, e.g., the papillary layer or the reticular layer.

Examples of skin disorders include psoriasis, psoriatic arthritis, dermatitis (eczema), e.g., exfoliative, allergic, or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia greata, pyoderma gangrenosum, vitiligo, pemphigoid (e.g., ocular cicatricial pemphigoid or bullous pemphigoid), urticaria, prokeratosis, rheumatoid arthritis that involves hyperproliferation and inflammation of epithelial-related cells lining the joint capsule; dermatitises such as seborrheic dermatitis and solar dermatitis; keratoses such as seborrheic keratosis, senile keratosis, actinic keratosis, photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; lichen planus; and keratitis.

In some embodiments, the disorder is psoriasis. The term “psoriasis” is intended to have its medical meaning, namely, a disease that afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis. Examples of psoriatic disorders include chronic stationary psoriasis, psoriasis vulgaris, eruptive (gluttate) psoriasis, psoriatic erythroderma, generalized pustular psoriasis (Von Zumbusch), annular pustular psoriasis, and localized pustular psoriasis.

Cellular Degenerative Disorders

Examples of cellular degenerative disorders include neurodegenerative disorders, muscular degenerative disorders, and neuromuscular degenerative disorders. Such degenerative disorders, typically characterized by a slowly progressive loss of function due to loss of certain group of cells, e.g., related neurons or muscle cells (e.g., myotubes), include Alzheimer's disease, Parkinson's disease, Huntington's disease, Amyotrophic Lateral Sclerosis, Multiple Sclerosis, torsions dystonia-idiopatic/symptomatic, musculorum deformans, spastic torticollis, blepharospasm, hereditary progressive dystonia, segmental dystonias and dyskinesias, olivo-pontocerebellar degeneration, hereditary ataxias, spinocerebellar degeneration, progressive bulbar palsy, acute idiopathic polyneuropathy, Charcot-Marie-Tooth disease, Rett syndrome, muscular dystrophies such as Duchenne Muscular Dystrophy, progressive muscular atrophy cachexia, and sarcopenia.

Methods of Treatment: Modulating Acheron-mediated Apoptosis

To treat cellular proliferative and/or differentiative disorders, apoptosis can be enhanced by increasing Acheron activity, e.g., by administering an agent that increases Acheron activity as described herein, e.g., an Acheron nucleic acid molecule, polypeptide, or fragment thereof, or an agent that increases CASK-C activity, e.g., a CASK-C polypeptide or nucleic acid, or an agent that decreases Ariadne activity, e.g., antisense nucleic acid, siRNA, ribozyme, inhibitory antibody, or dominant negative targeting Ariadne.

Conversely, to treat cellular degenerative disorders, apoptosis can be inhibited by decreasing Acheron activity. In such methods, inhibition of apoptosis can be achieved by decreasing Acheron activity, for example, by administration of an agent that decreases Acheron activity as described herein, e.g., an Acheron antisense nucleic acid, siRNA, ribozyme, inhibitory antibody, dominant negative, or an agent that increases Ariadne activity, e.g., an Ariadne polypeptide or nucleic acid, or an agent that decreases CASK-C activity, e.g., antisense nucleic acid, siRNA, ribozyme, inhibitory antibody, or dominant negative targeting CASK-C, e.g., a fragment of CASK-C that interacts with Acheron as described herein (see Example 13).

Inhibition of apoptosis can also be achieved by administration of an agent that decreases Acheron activity by preventing or inhibiting translocation of Acheron to the nucleus, e.g., antibodies; dominant negative forms of Acheron, e.g., tAch or, alternatively, a peptide comprising: a region of SEQ ID NO:4 corresponding to one or more of the following: residues 1-33; residues 34-491; all or part one or more of the Acheron functional domains; small molecules, e.g., that interfere with Ach-CASK-C, Ach-Ariadne, or Ach-parkin binding; kinase inhibitors, e.g., a kinase inhibitor that decreases phosphorylation of one or more phosphorylation sites on Acheron, e.g., one or more phosphorylation sites in the N-terminus; or a dominant negative form of CASK-C, e.g., a peptide comprising amino acids 1-304 of CASK-C.

As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. Therapeutic agents include, for example, proteins, nucleic acids, small molecules, peptides, antibodies, siRNAs, ribozymes, and antisense oligonucleotides.

With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype” or “drug response genotype”). Thus, another aspect of the invention provides methods for tailoring an individual's prophylactic or therapeutic treatment with either the Acheron molecules of the present invention or Acheron modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for preventing in a subject, a disease or condition associated with aberrant cellular differentiation, proliferation, or degeneration, by administering to the subject Acheron or an agent that modulates Acheron expression or at least one Acheron activity. Subjects at risk for a disease associated with aberrant cellular differentiation, proliferation, or degeneration or activity can be identified by, for example, any or a combination of diagnostic or prognostic assays known in the art. Administration of a prophylactic agent can occur prior to the manifestation of symptoms characteristic of the Acheron aberrance, such that a disease or disorder is prevented or, alternatively, delayed in its progression. Depending on the type of Acheron aberrance, for example, an Acheron polypeptide or nucleic acid, an Acheron agonist, or an Acheron antagonist agent can be used for treating the subject. The appropriate agent can be determined, e.g., based on screening assays described herein. Experiments with C₂C₁₂ cells indicate that Acheron regulates integrin expression. This means that alterations in integrin function could play a major role in the metastatic potential of cancer cells. Normal cells initiate apoptosis when they lose contact with the substrate. This phenomenon, which is termed anoikis (homelessness) is a major defensive mechanism for preventing metastasis. If cells can overcome anoikis, they have much greater freedom to grow out of the plane of the tissue and colonize other distant tissues. Activation or retardation of Acheron can impact on this process.

The Acheron molecules can act as novel diagnostic targets and therapeutic agents for controlling one or more of cellular proliferative and/or differentiative disorders, and disorders associated with cellular degeneration. For example, Acheron expression was examined by semi-quantitative reverse transcription PCR in 60 different cell lines representing the majority of human cancers (NCI60 Cell Lines, maintained by the National Cancer Institute; Scherf et al. (2000) Nat. Genet. 24(3):236-44); Acheron was expressed in almost all of the lines (see Table 1).

TABLE 1
Relative Acheron Expression in Human Tumor
Derived Cell Lines

| | | Relative |
|---|---|---|
| Cell line | Origin | hAch expression |
| | Lung | |
| NCI-H23 | non-small cell | + |
| | adenocarcinoma | |
| NCI-H226 | squamous cell carcinoma | + |
| NCI-H322M | bronchioalveolar carcinoma | + |
| NCI-H460 | large cell anaplastic | ++ |
| | carcinoma | |
| NCI-H522 | non-small cell | ++ |
| | adenocarcinoma | |
| A549/ATCC | adenocarcinoma | +++ |
| HOP-62 | adenocarcinoma | ++ |
| HOP-92 | large cell anaplastic | + |
| | carcinoma | |
| EKVX | adenocarcinoma | ++++ |
| | Ovary | |
| OVCAR-3 | adenocarcinoma | ++ |
| OVCAR-4 | adenocarcinoma | ++ |
| OVCAR-5 | adenocarcinoma | ++ |
| OVCAR-8 | adenocarcinoma | ++ |
| IGROV-1 | adenocarcinoma | + |
| SK-OV-3 | adenocarcinoma | ++ |
| | CNS | |
| SNB-19 | glioblastoma multiforme | +++ |
| SNB-75 | astrocytoma | +++ |
| U251 | glioblastoma multiforme | ++++ |
| SF-268 | glioblastoma multiforme | ++ |
| SF-295 | glioblastoma multiforme | ++ |
| SF-539 | glioblastoma multiforme | + |
| Cell line | Origin | Acheron expression |
| | Lymphoid/Haemopoietic | |
| | tissues | |
| CCRF-CEM | acute lymphoblastic | |
| | leukemia | |
| K-562 | chronic myelogenous | + |
| | leukemia | |
| MOLT-4 | acute T lymphoblastic | ++ |
| | leukemia | |
| HL-60 (TB) | acute promyelocytic | +/ |
| | leukemia | |
| RPMI 8226 | multiple myeloma | ++ |
| SR | large cell immunoblastic | |
| | lymphoma | |
| | Prostate | |
| DU-145 | adenocarcinoma | +/ |
| PC-3 | adenocarcinoma, grade IV | ++ |
| | Colon | |
| HT-29 | adenocarcinoma, grade II | ++ |
| HCC-2998 | adenocarcinoma | +/ |
| HCT-116 | adenocarcinoma | ++ |
| SW-620 | adenocarcinoma, | +++ |
| | Dukes' type C | |
| COLO 205 | adenocarcinoma, | ++ |
| | Dukes' type D | |
| HCT-15 | adenocarcinoma, | +++ |
| | Dukes' type C | |
| KM12 | adenocarcinoma | |
| | Kidney | |
| UO-31 | renal adenocarcinoma | ++ |
| SN12C | renal adenocarcinoma | + |
| A498 | renal adenocarcinoma | ++ |
| CAKI-1 | renal adenocarcinoma | ++ |
| RXF 393 | renal adenocarcinoma | |
| ACHN | renal adenocarcinoma | ++ |
| 786-0 | renal adenocarcinoma | ++ |
| TK-10 | renal adenocarcinoma | +/ |
| | Melanoma | |
| LOX IMVI | amelanotic | ++ |
| MALME-3M | melanoma (metastatic) | +/ |
| SK-MEL-2 | melanoma (metastatic) | |
| SK-MEL-5 | melanoma | +/ |
| SK-MEL-28 | melanoma | +/ |
| UACC-62 | melanoma | + |
| UACC-257 | melanoma | ++ |
| M14 | melanoma | +/ |
| | Breast | |
| MCF7 | adenocarcinoma | +++ |
| NCI/ADR-RES | adenocarcinoma | +++ |
| HS 578T | ductal adenocarcinoma | ++++ |
| MDA-MB-231/ATCC | adenocarcinoma | ++++ |
| MDA-MB-435 | adenocarcinoma | ++ |
| BT-549 | ductal adenocarcinoma | +++ |
| | (metastasis) | |
| T-47D | ductal adenocarcinoma | ++ |

Some disorders may be associated, at least in part, with an abnormally high level of Acheron gene product, or by the presence of an Acheron gene product exhibiting abnormally high activity. As such, the reduction in the level and/or activity of such gene products would bring about the amelioration of disorder symptoms. Such disorders are associated with cellular degeneration, e.g., neurodegenerative disorders, or muscular degenerative disorders. Other disorders, such as disorders associated with aberrant cellular proliferation or differentiation, may be associated, at least in part, with an abnormally low level of Acheron gene product, or by the presence of an Acheron gene product exhibiting abnormally low activity. An increase in the level and/or activity of such gene products would bring about the amelioration of disorder symptoms.

As discussed, successful treatment of disorders associated with aberrant cellular differentiation, proliferation, or degeneration can be by techniques that serve to modulate the expression or activity of Acheron gene products. For example, compounds, e.g., an agent identified using an assays described herein, that enhances Acheron activity, can be used in accordance with the invention to prevent and/or ameliorate symptoms of cellular proliferative disorders. Such molecules can include, but are not limited to, Acheron nucleic acids or active fragments thereof, peptides, phosphopeptides, small organic or inorganic molecules, agents that decrease Ariadne activity (e.g., antisense, siRNA, and ribozyme molecules, dominant negatives, peptides, phosphopeptides, small organic or inorganic molecules, or antibodies), or agents that increase CASK-C activity (e.g., CASK-C nucleic acids or proteins or active fragments thereof).

In addition, compounds, e.g., an agent identified using an assays described above, that inhibits Acheron activity, can be used in accordance with the invention to prevent and/or ameliorate symptoms of cellular degenerative disorders. Such molecules can include, but are not limited to, dominant negative variants of Acheron, peptides, phosphopeptides, small organic or inorganic molecules, or antibodies (including, for example, polyclonal, monoclonal, humanized, anti-idiotypic, chimeric or single chain antibodies, and Fab, F(ab′)₂ and Fab expression library fragments, scFV molecules, and epitope-binding fragments thereof), agents that increase Ariadne activity (e.g., Ariadne nucleic acids or proteins or active fragments thereof), or agents that decrease CASK-C activity (e.g., antisense, siRNA, and ribozyme molecules, dominant negatives, peptides, phosphopeptides, small organic or inorganic molecules, or antibodies).

Further, antisense (e.g., morpholino oligonucleotides), siRNA, and ribozyme molecules as described herein that inhibit expression of the Acheron gene can also be used in accordance with the invention to reduce the level of Acheron expression, thus effectively reducing the level of Acheron activity. Still further, triple helix molecules can be utilized in reducing the level of Acheron activity.

Another method by which nucleic acid molecules may be utilized in treating or preventing a disease characterized by aberrant cellular differentiation, proliferation, or degeneration is through the use of aptamer molecules specific for Acheron protein. Aptamers are nucleic acid molecules having a tertiary structure, which permits them to specifically bind to protein ligands (see, e.g., Osborne, et al. Curr. Opin. Chem Biol. 1:5-9 (1997); and Patel, Curr Opin Chem Biol 1:32-46 (1997). Since nucleic acid molecules may in many cases be more conveniently introduced into target cells than therapeutic protein molecules may be, aptamers offer a method by which Acheron protein activity may be specifically decreased without the introduction of drugs or other molecules that may have pluripotent effects.

Antibodies can be generated that are both specific for Acheron and that reduce Acheron activity. Such antibodies can, therefore, be administered in instances whereby negative modulatory techniques are appropriate for the treatment of Acheron disorders. Antibodies and methods of making them are known in the art and described herein.

In circumstances wherein injection of an animal or a human subject with an Acheron protein or epitope for stimulating antibody production is harmful to the subject, it is possible to generate an immune response against Acheron through the use of anti-idiotypic antibodies (see, for example, Herlyn, Ann Med 31:66-78 (1999); and Bhattacharya-Chatterjee and Foon, Cancer Treat Res. 94:51-68 (1998). If an anti-idiotypic antibody is introduced into a mammal or human subject, it should stimulate the production of anti-anti-idiotypic antibodies, which should be specific to the Acheron protein. Vaccines directed to a disease characterized by Acheron expression may also be generated in this fashion.

In instances where the target antigen is intracellular and whole antibodies are used, internalizing antibodies can be used. Lipofectin or liposomes can be used to deliver the antibody or a fragment of the Fab region that binds to the target antigen into cells. Where fragments of the antibody are used, the smallest inhibitory fragment that binds to the target antigen can be used. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al. Proc. Natl. Acad. Sci. USA 90:7889-7893 (1993).

In another aspect, the invention provides another method for the treatment of diseases associated with aberrant cellular degeneration by the transplantation of cells exhibiting decreased Acheron activity, i.e., cells expressing Acheron antisense or dominant negative forms, shRNAs, or ribozymes, as described herein. Generally, the cells can be stem cells or partially differentiated cells, e.g., myoblasts or neural stem cells. As one example, muscular dystrophy can be treated by a method described herein including transplanting into a subject having muscular dystrophy myoblasts lacking Acheron activity or having decreased activity. As a second example, demyelinating disorders can be treated by transplanting Schwann cells or Schwann cell progenitors lacking Acheron activity or having decreased activity. The cells can be transplanted directly into an area that is undergoing degeneration. The cells can be autologous, e.g., taken from the intended recipient, or heterologous, e.g., taken from a suitable donor, e.g., an immune-matched donor. In some embodiments, the cells express an additional ectopic gene or genes, e.g., genes to further enhance survival of the transplanted cells, or genes to treat the intended recipient, e.g., the dystrophin gene.

The identified compounds that inhibit Acheron gene expression, synthesis and/or activity can be administered to a patient at therapeutically effective doses to treat, e.g., ameliorate, symptoms associated with cellular degeneration. A therapeutically effective dose refers to that amount of the compound sufficient to result in amelioration of symptoms of the disorders. Toxicity and therapeutic efficacy of such compounds can be determined by standard pharmaceutical procedures as described above.

The data obtained from cell culture assays and animal studies can be used in formulating a range of dosage for use in humans. The dosage of such compounds can lie within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays may utilize antibody mimics and/or “biosensors” that have been created through molecular imprinting techniques. A compound that modulates Acheron activity is used as a template, or “imprinting molecule,” to spatially organize polymerizable monomers prior to their polymerization with catalytic reagents. The subsequent removal of the imprinted molecule leaves a polymer matrix that contains a repeated “negative image” of the compound and is able to selectively rebind the molecule under biological assay conditions. A detailed review of this technique can be seen in Ansell et al Current Opinion in Biotechnology 7:89-94 (1196) and in Shea, Trends in Polymer Science 2:166-173 (1994). Such “imprinted” affinity matrixes are amenable to ligand-binding assays, whereby the immobilized monoclonal antibody component is replaced by an appropriately imprinted matrix. An example of the use of such matrixes in this way can be seen in Vlatakis et al Nature 361:645-647 (1993). Through the use of isotope-labeling, the “free” concentration of compound that modulates the expression or activity of Acheron can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiber-optic devices, in turn allowing the dose in a test subject to be quickly optimized based on its individual IC₅₀. One example of such a “biosensor” is discussed in Kriz et al Analytical Chemistry 67:2142-2144 (1995).

Cell Transplantation

Recently, methods of cell transplantation have been developed for the treatment of disease. In its most basic form, this method involves transplanting either stem cells or partially differentiated cells into damaged tissues and organs with the hope that they will engraft and effect repair. For example, sympathetic neurons have been successfully used to replace missing dopaminergic neurons in Parkinson's Disease (Nakao et al (2001) J. Neurosurg. 95(2):275-84). Indeed, the current interest in therapeutic cloning and stem cells arises from the promise of cell transplantation to repair damaged organs.

Much of the excitement over stem cell biology arises from its great potential to repair or rejuvenate aging or damaged tissues (Mombaerts, Proc. Natl. Acad. Sci. USA 100 Suppl 1:11924-5 (2003); Roccanova and Ramphales, Tissue Cell. 35(1):79-81 (2003)). Embryonic stem (ES) cells are totipotent cells that arise early in embryogenesis and have the capacity to generate all the different tissues in the body. This capability has captured the imagination of both researchers and the general public as a panacea for treating human disease.

The goal of therapeutic cloning is to create progenitor cells that become committed to a specific lineage to provide specialized cells that can be employed to repair damaged tissues in patients. As examples, ES cells can be used to create new pancreatic beta cells to treat type I diabetes (Hori et al., Proc. Natl. Acad. Sci. USA 99(25):16105-10 (2002)) or new neurons to reverse the ravages of Parkinson's or Alzheimer's diseases (Ostenfeld and Svendsen, Adv. Tech. Stand. Neurosurg. 28:3-89 (2003); Borlongan et al. Drug Discov Today 7(12): 674-82 (2002)).

One strategy is to use adult-derived stem cells (Hirai, Hum. Cell. 15(4):190-8. 2002). These cells offer a number of advantages over ES cells. They can be autologous, i.e., harvested from the patient themselves, thus precluding issues of rejection. In addition, they are often restricted to specific lineages, which reduces the potential that these mitotically competent cells will give rise to neoplasms. In the area of treating cardiovascular disease, several recent papers have documented the use of autologous muscle-derived satellite cells to repair myocardial dysfunction in humans (Hagege et al., Lancet. 361(9356):491-2 (2003); Menasche et al., J. Am. Coll. Cardiol. 41(7):1078-83 (2003)). Patients receiving these ectopic cells displayed engraftment and improved left ventricular ejection fraction.

The best-studied stem cell population used for treating human disease is obtained from bone marrow, which can be used as part of the treatment regimen for a variety of lymphomas and leukemias.

One issue associated with cell transplantation is the propensity for transplanted cells to undergo apoptosis. Blocking this natural tendency to undergo apoptosis could be blocked should results in increases in both survival and clinical benefit. The methods described herein can be used to enhance the success rate of cell-based therapy using practically any cell type, as inhibiting the action of Acheron will enhance the survival of the cells before and after transplantation. More cells surviving means greater transplant efficiency, so fewer cells need to be provided, and fewer cells need to be transplanted.

One of the technical benefits of the methods described herein is that they can shorten the time between harvesting cells and reintroducing them back into the patient since more of the cells generated in vitro will survive when introduced in vivo. Patients will presumably benefit from speedier treatment since it will: 1) reduce hospital stay and associated costs; 2) reduce ischemic and other secondary damage to the heart (or other organs); 3) reduce the risk that the cultured cells will acquire either infections or mutations; and 4) reduce the costs of generating patient-specific autologous grafts by reducing the labor and related costs of long term cell culture.

Thus, the invention includes methods for enhancing the success rate of cell-based therapy including transplanting cells, e.g., autologous muscle cells, that express exogenous Acheron (with or without other exogenous genes), or that have reduced levels of Acheron expression or activity, e.g., cells that express or have been treated with an inhibitor of Acheron expression or activity, e.g., an Acheron antibody, antisense, siRNA, or dominant negative as described herein.

The methods include providing cells having reduced or no Acheron activity, e.g., cells wherein the Acheron activity has been inhibited, e.g., by one or more of the methods described herein, and transplanting the cells into the subject. For example, in the case of a subject having a disorder associated with demyelination, such as multiple sclerosis or spinal injury, the myelin sheath can be regenerated by transplanting a population of myelin-producing cells, e.g., oligodendrocytes or oligodendrocyte progenitor cells, having reduced or no Acheron activity, into one or more appropriate sites in the subject. For example, a number of cells, e.g., about 10³, 10⁴, 10⁵, 10⁶ or more cells can be injected at one or more sites. In the case of a subject having a disorder associated with muscular degeneration, the muscle can be regenerated by transplanting a population of myoblasts having reduced or no Acheron activity (e.g., cells that express or have been treated with an inhibitor of Acheron expression or activity, e.g., an Acheron antibody, antisense, siRNA, or dominant negative as described herein) into one or more appropriate sites in the subject. For example, a number of myoblasts e.g., about 10³, 10⁴, 10⁵, 10⁶ or more cells can be injected at one or more sites.

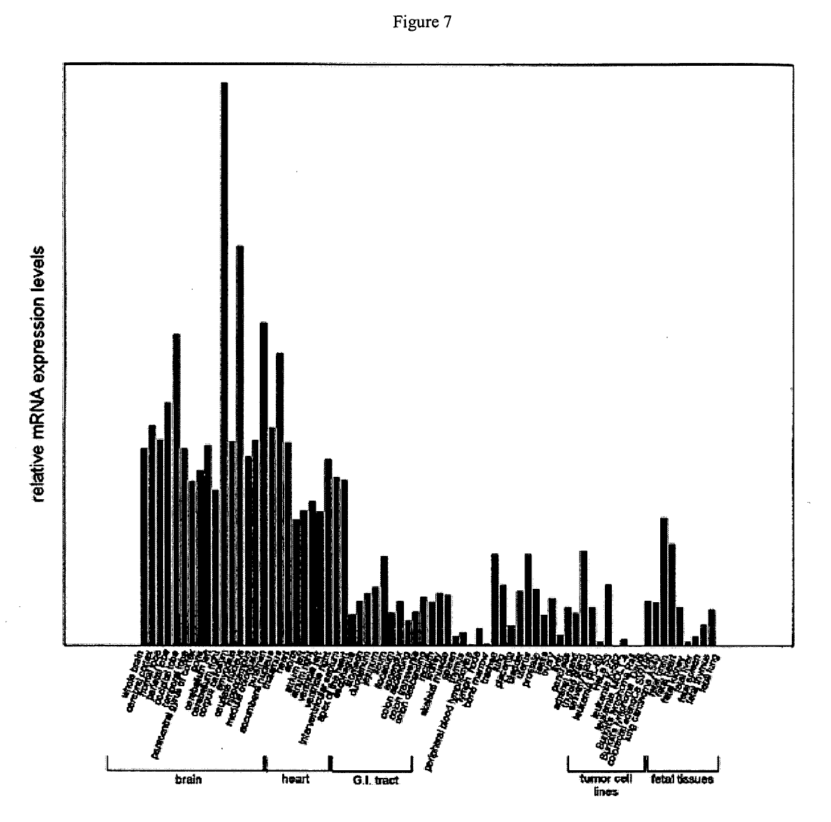

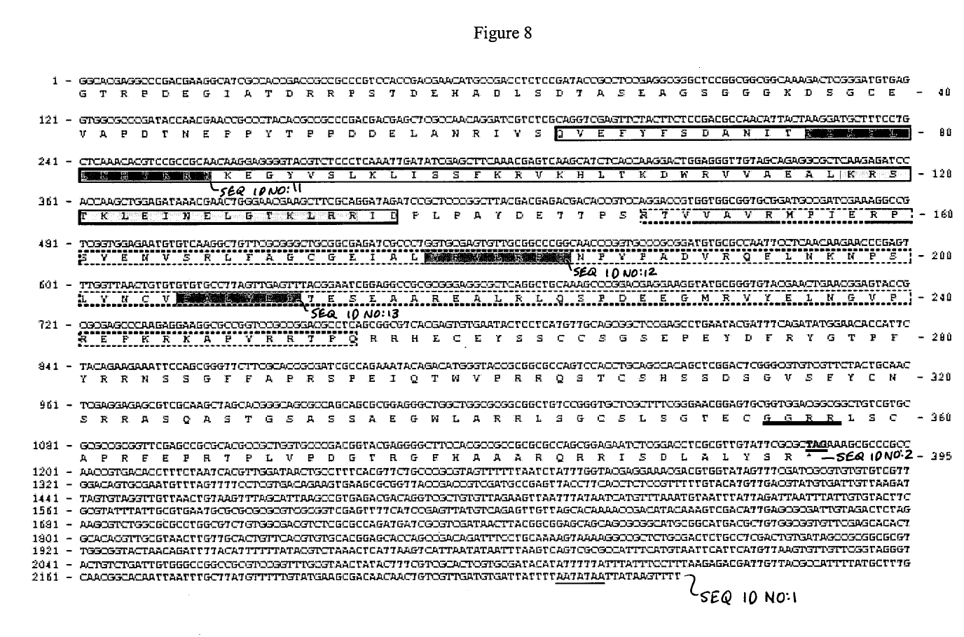

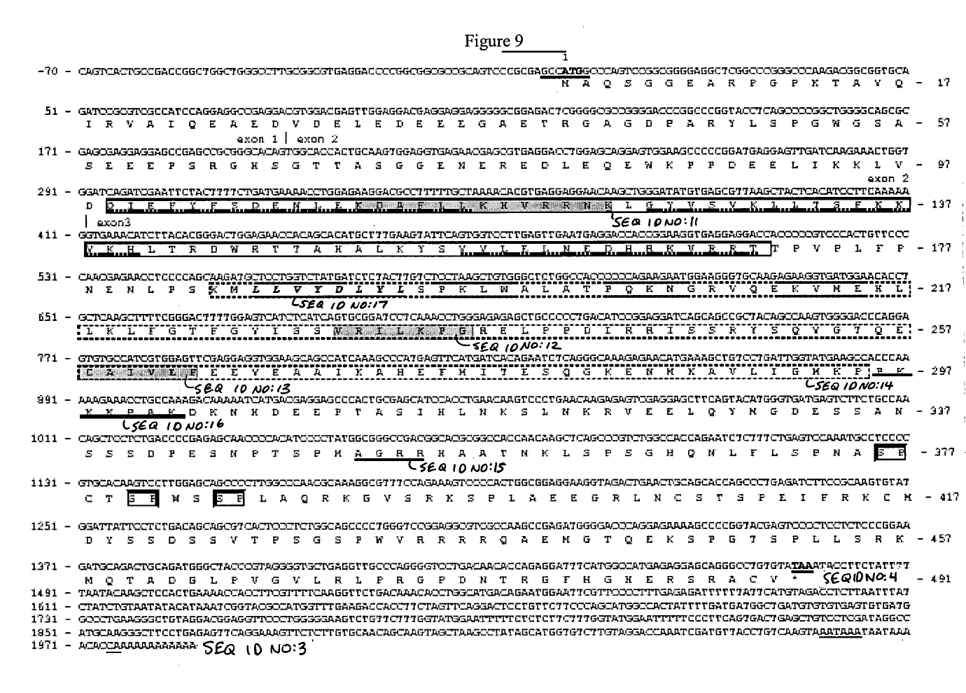

Seventy-three different human tissues were screened at the RNA level via dot blot to determine the distribution of Acheron (see FIG. 7). The highest levels of expression were found in the nervous system. Within the CNS, the tissues with the highest levels of Acheron was the corpus collosum. This is a major fiber tract that connects the hemispheres in the brain. The only cell type found in significant levels in that tissue are oligodendrocytes, cells responsible for providing the myelin wrapping of neurons. This suggests that Acheron may be required for their normal function. Demyelinating diseases like Multiple Sclerosis are a major clinical problem and factors that influence the myelination/demyelination of axons are a major focus. Based on in situ hybridization studies with rats, there are extremely high levels of Acheron mRNA expression in the spinal cord. Remyelination is a key factor in the effective functioning of spinal motor neurons after spinal cord injury. Thus, the invention includes methods of treating a subject having a degenerative disorder.

Genetic Therapy

A significant proportion of human diseases arise when germ-line or somatic mutations produce aberrations in protein structure and function. These defective proteins in turn lead to perturbations in developmental or homeostatic processes and subsequent pathology. Experimental data obtained from both cell culture and animal models have demonstrated that in certain cases, correction of these genetic defects restores normal physiological responses and abrogates pathology. These results have lead to an intense focus on developing strategies for exploiting gene therapy for the treatment of human disease.

One of the problems with exploiting gene therapy is finding methods that allow the desired DNA sequences to enter a cell and direct gene expression.

One strategy for gene therapy is to use transplanted cells engineered to produce foreign hormones or factors, such as factor IX, erythropoietin, growth hormone, proinsulin, and the granulocyte colony stimulating factor-I using methods known in the art. Any of these cells can also be engineered to express an inhibitor of Acheron expression or activity, e.g., an antisense, siRNA, or dominant negative form of Acheron, to enhance viability of the transplanted cells.

Stem cells can be engineered, e.g., to carry a desired therapeutic gene, and can include 5′ regulatory sequences, e.g., to express ectopic genes for use in gene therapy, before reintroduction into the patient. For example, the cells can be engineered to carry a gene that decreases the expression or activity of Acheron in the cell, e.g., an antisense, siRNA, or dominant negative form of Acheron, alone or in addition to a therapeutic gene.

There are several advantages associated with cell transplantation over viral vectors for gene therapy. The first is that there is no practical upper limit to the size of the DNA that can be introduced. In fact, it is possible to engineer these cells to carry supernumerary chromosomes encoding a large number of distinct genes, complete with their normal regulatory sequences (Saffery and Choo, J. Gene Med. 4(1):5-13 (2002)).

Myoblast Development and Transplantation

Mature skeletal muscles contain a quiescent pool of stem cells known as satellite cells. Satellite cells received their name because of their location outside muscle fibers, but under the sarcolemma. Satellite cells remain arrested in the Go phase of the cell cycle until they become activated by a variety of local signals following skeletal muscle injury. These cells then re-enter the cell cycle and produce large numbers of progeny, some of which can fuse with the damaged muscle fibers and effect repair, while others exit the cell cycle to reconstitute the satellite pool. In normal individuals, satellite cells persist throughout life and can affect repair even in aged individuals. However, this is not true for patients with Duchenne Muscular Dystrophy.

Satellite cells can be readily isolated from most donors by performing a muscle biopsy and culturing the tissue in a medium rich in growth factors, e.g., as described in Decary et al., Hum. Gene. Ther. 8(12):1429-38 (1997), and in Blau et al., Exp. Cell. Res. 144(2): 495-503 (1983). In vitro, the satellite cells become activated and migrate away from the damaged muscle fibers. These activated cells are referred to as muscle precursor cells (MPCs) and they can be cultured for many generations in vitro. In fact, these non-transformed stem cells can proliferate well beyond the Hayflick limit that restricts the use of most other cells derived from the body, a factor that further facilitates their use. These expanded cells can be used as is for tissue repair or be engineered, e.g., to carry a desired gene, and 5′ regulatory sequences, e.g., to express ectopic genes for use in gene therapy. These ectopically expressed proteins can be used to enhance muscle function, such as dystrophin in Muscular Dystrophy (Skuk et al., J. Neuropathol. Exp. Neurol. 59(3):197-206 (2002)), or instead secrete factors systemically such as factor IX (Chen et al., Hum. Gene. Ther. 9(16):2341-51 (1998)) and granulocyte colony stimulating factor-1 (Moisset et al., Hum. Gene Ther. 11(9):1277-88 (2000)). Once the desired population of cells has been harvested in vitro they can be injected back into the skeletal muscle in vivo. These engineered MPCs can then fuse with mature muscle fibers and reconstitute the satellite pool, and express the desired gene(s) (e.g., an Acheron-inhibiting gene, e.g., a dominant negative). Thus, these MPCs are suitable for use in myoblast transplantation methods.

While satellite cells seem to be almost ideal vehicles for gene therapy and tissue repair, experimental studies have demonstrated that very few ectopic myoblasts survive and fuse with host muscle fibers (Gussoni et al., Nat. Med. 3(9):970-7 (1997); Fan et al., Muscle Nerve 19(7):853-60 (1996); Qu et al., J. Cell. Biol. 142(5):1257-67 (1998)). While data from different laboratories indicate fundamentally different levels of cell loss following transplantation, even in the best-case scenario the present inventors reported a >70% loss of the initial transplanted pool (Skuk et al., (2002), supra). Even though subsequent mitosis may have increased the population of ectopic cells, this is still a troubling statistic for two reasons. First, if these condemned cells survived, then the number of cells that could contribute to future repair and satellite cell formation would be dramatically increased. Second, and perhaps more troubling, is the observation that that activated satellite cells are very heterogeneous with regard to their phenotypic properties (Qu et al., (1998), supra). Selective loss of specific sub-populations could have real clinical consequences, especially they are cells that are predisposed to divide, migrate or fuse.

If the natural tendency for transplanted cells to undergo apoptosis could be blocked, there should be a concomitant increase in both survival and clinical benefit. As demonstrated herein (see Example 3, below), expression of a dominant-negative form of Acheron allows myoblasts to survive in the absence of trophic support, thus making it an ideal target for developing cell-based therapies. Thus, the invention includes methods and compositions to block apoptosis in a manner that facilitates the survival and incorporation of transplanted myoblasts, by inhibiting Acheron activity. For example, the satellite cells can be engineered to express a gene that decreases the expression or activity of Acheron in the cell, e.g., an antisense, siRNA, or dominant negative form of Acheron. Alternatively, the cells can be treated with an inhibitor of Acheron activity or expression prior to transplantation such as an Acheron antisense (e.g., morpholino oligonucleotide), antibody, siRNA, or dominant negative. Thus, the cells have reduced levels of Acheron expression or activity.

As noted above, one of the attractive features of using an RNAi or antisense approach (e.g., morpholino oligos, Heasman, Dev. Biol. 243:209-14(2002); Iversen, Curr. Opin. Mol. Ther. 3:235-8 (2001); Summerton, Biochim. Biophys. Acta. 1489:141-58 (1999)) is that foreign genes are not introduced into the cells prior to re-introduction into the body. This has the advantage that the effects of inhibiting Acheron should be transient. Since Acheron inhibits both death and differentiation, this is a problem. If Acheron were only transiently inhibited, the cells would initially survive and then over time acquire the capacity to differentiate or fuse with other cells. Once either of these steps happen, they will activate survival programs and not need the benefits of Acheron.

Although these methods are described in detail herein in the context of myoblast transplantation, they are equally applicable to other transplant scenarios, including transplantation of neural cells to treat degenerative conditions; hematopoietic cells to treat hematologically-related conditions; and fibroblast cells to treat skin or other conditions. The methods of treating a subject having a degenerative disorder include providing cells having reduced or no Acheron activity, e.g., cells wherein the Acheron activity has been inhibited, e.g., by a methods described herein, and transplanting the cells into the subject. For example, in the case of a subject having a disorder associated with demyelination, such as multiple sclerosis or spinal injury, the myelin sheath can be regenerated by transplanting a population of myelin-producing cells, e.g., oligodendrocytes or oligodendrocyte progenitor cells, having reduced or no Acheron activity, into one or more appropriate sites in the subject. For example, a number of cells, e.g., about 10³, 10⁴, 10⁵, or 10⁶ cells can be injected at one or more sites. In the case of a subject having a disorder associated with muscular degeneration, the muscle can be regenerated by transplanting a population of myoblasts having reduced or no Acheron activity into one or more appropriate sites in the subject. For example, a number of myoblasts e.g., about 10³, 104, 105, or 10⁶ cells can be injected at one or more sites.

Muscular Dystrophy

While myoblast-based gene therapy can be used to treat a variety of human illness, its primary use clinically has been directed towards the treatment of Duchenne Muscular Dystrophy (DMD). DMD is a hereditary disease that manifests symptoms beginning around age 5 and is characterized by progressive muscle weakness. By age 10 patients are usually confined to a wheel chair and by age 18 upper extremity weakness makes even control of an electric wheelchair or computer mouse difficult. By this time, respiratory muscle weakness requires nighttime and then full time mechanical ventilation. Most patients die of respiratory problems or secondary heart disease between 17 and 24 years old.

Because of the large size of the dystrophin gene, researchers have had to create truncated mini-genes for use with viral vectors. While some positive data have been obtained, this approach has been disappointing in clinical applications (Roberts and Dickson, Curr. Opin. Mol. Ther. 2002 August; 4(4):343-8 (2002). The alternative approach of myoblast transfer is more promising because, by fusing with diseased muscle fibers, wild-type myoblasts can contribute both the normal gene and its 5′ regulatory sequences. Presumably the continued expression of normal Dys in these fibers will protect them from further damage. In addition, donor myoblasts generate additional satellite cells and the potential to repair future muscle damage.

A suitable model for DMD is the C57BL10J mdx/mdx (mdx) mouse (Vilquin et al., J. Cell Biol. 131(4):975-88 (1995); Cox et al., Nature. 364(6439):725-9 (1993)) which lacks subsarcolemmal Dys because of a mutation in position 3185 of the Dys gene (Sicinski et al. Science. 244(4912):1578-80 (1989)). While DMD has an early onset in humans, the mdx mice exhibit few of the clinical symptoms of DMD before 18 months of age. Beyond this time however, mdx mice progressively exhibit a dystrophic phenotype and almost all muscles, including cardiac and some smooth muscles, are invaded by fibrotic tissue and become atrophic. Respiratory muscles are especially affected and mdx mice exhibit a shorter life span than do normal mice. Depending on their age and strain, 0.1 to 1% of the muscle fibers in mdx mice are revertent, and express a truncated form of Dys.

There is already some evidence supporting the beneficial effects of myoblast transplantation (MT). When mdx mice were subjected to eccentric exercise one month following MT, muscle lengthening contractions known to produce strain leading to muscle damage, myofiber damage was observed only in Dys-negative fibers but not in the Dys-positive fibers resulting from the MT (Partridge et al., Nat. Med. 4(11): p. 1208-9 (1998). Thus, MT protected the muscle tissue of mdx mice from the mechanical strain, which serves as the trigger for myofiber necrosis in DMD. Morgan et al. (Morgan et al., J. Neurol. Sci. 115(2):191-200 (1993) observed that the number of Dys-positive fibers did not change from 35 to 250 days after MT, while the number of Dys-negative fibers decreased progressively. This was attributed to the protective role of the donor Dys.

The present invention provides methods for the treatment of muscular dystrophy, comprising administering cells having reduced Acheron activity, e.g., as described herein, to a subject having muscular dystrophy. Where the cells are autologous, the cells having reduced Acheron activity can also express a dystrophin gene or biologically active fragment thereof, e.g., as known in the art.

The invention is further described in the following examples, which do not limit the scope of the invention described in the claims.

### EXAMPLES

Materials and Methods

Cell Culture

C₂C₁₂ cells were cultured in growth medium (GM) consisting of Dulbecco's modified Eagle medium (DMEM) supplemented with 15% (vol/vol) fetal calf serum (FCS), 5% of fetal bovine serum (FBS Atlanta Biologicals, Norcross, Ga.) and 100 units/ml of penicillin-streptomycin (Gibco). To induce the differentiation, subconfluent cultures were shifted to differentiation medium (DM) consisting of DMEM supplemented with 2% horse serum and 100 units/ml of penicillin-steptomycin.

Transfection

LipofectAmine™ (Gibco) was used to transfect C₂C₁₂ cells with hAch, tAch, As-Ach or empty vector according to the manufacturer's protocol. Antibiotic-resistant stable transformants were selected in GM with G418 (500 μg/ml) or puromycin (3 μg/ml). About 10 monoclonal lines were randomly chosen from each transfection, and further analyses were performed with typical individual lines.

In some experiments, transfected cells were re-transfected with pBabe-hygro-MyoD or empty pBabe-hygro vector and stable cells selected with 250 μg of hygromycin B/ml (Sigma). Similarly, pBabe-puro-tAch or pBabe-puro-As-Ach were stably transfected into neomycin-resistant cells over-expressing full-length Acheron and selected using puromycin (3 μg/ml).

Western Blots

C₂C₁₂ cells were collected at various time in GM and DM and extracted in Laemmi buffer without DTT or P-ME. Protein concentration was determined by BSA assay (Pierce) and then DTT or P-ME was added to the sample. 20 μg of protein for each sample was fractionated by 10% SDS-PAGE, transferred to Immobilon P membrane (Millipore), and reacted with the primary antibody. Horseradish peroxidase-labeled secondary antisera were detected with the enhanced chemiluminescence (ECL) kit (Amersham Pharmacia) and X-ray film (Eastman Kodak).

Primary antibodies used included tAch (1:2,000), anti-FLAG monoclonal antibody (M5 1:500, Sigma), MyoD (1:500, Pharmingen, or, 1:200, C-20, Santa Cruz), Myf5 (1:200, C-20, Santa Cruz), Bcl-2 (1:400, Santa Cruz) and Bax (1:800, Oncogene).

Immunocytochemistry Staining

C₂C₁₂ cells were grown on 12-well plates or coverslips (Fisher Scientific) and then fixed in freshly made acetone and methanol (1:1 V/V) for 1 minutes (myoblasts) or 3 minutes (myotubes) at room temperature. The fixed cells were air-dried and rehydrated in phosphate-buffered saline (PBS). Alternatively, cells were fixed with 4% paraformaldehyde in PBS for 20 minutes at room temperature, washed twice with PBS and permeabilized with 0.3% Triton X-100 in PBS for 10 min. After three washes with PBS, cells were incubated with the primary antibody in PBST containing bovine serum albumin (10 mg/ml) at room temperature for 1-2 hours or at 4° C. overnight. Appropriate biotin-labeled secondary antibodies and horseradish peroxidase-conjugated avidin (Vector Laboratories) were used for detection according to the manufacturer's protocol. Primary antibodies included: tAch (1:400); desmin (1:200, polyclonal antibody from Sigma), myogenin (F5D, 1:50) and myosin heavy chain (MF20, 1:100, Development Studies Hybridoma Bank).

Cell Death Rate Determination by Trypan Blue Assay

Cells were incubated in DM for 24 or 48 hours and then trypsinized, resuspended in DMEM and stained with 1 volume of 0.1% Trypan Blue Stain (Sigma) according to the manufacturer's protocol. The percentage of cell death was determined by counting each cell line in triplicate.

Yeast Strain and Manipulation

Saccharomyces cerevisiae yeast strain Y190 and CG1945 (Clontech Matchmaker GAL4 Two-Hybrid User Manual) were used in the library screening. Standard microbiological techniques and media were performed on the growth of the strains. Media designations are as follows: YPD is YP (yeast extract plus peptone) medium with 2% glucose. SD media contains 2% glucose and a DO Supplement lacking the appropriate nutrient (e.g. SD/-ura/-trp/-leu media lacks uracil, tryptophan and leucine). Plasmid DNA was introduced into yeast by standard LiOAc-mediated transformation.

Yeast Two-Hybrid Screen

The known Acheron coding region (lacking a small part of N-terminal) was amplified by PCR with primers containing restriction sites and was cloned in frame to SalI site of pAS2-1 to form pAS2-1-Acheron as the bait. pAS2-1-Acheron was transformed into yeast CG1945 and then the expression of the fusion protein was detected by Western blotting using Acheron antibody (Larry Schwartz's lab). No autonomous activation was observed for bait pAS2-1-Acheron. Mouse 17-day embryo cDNA library (Clontech) was amplified by plating the library directly on LB/amp plates at high enough density. The colonies were collected and pACT2 library plasmids were isolated by Qiagen plasmid DNA purification (Mega) Kit. Strain CG1945 containing pAS2-1-Acheron was transformed with pACT2 library plasmids and a total of 4.8×10⁶ independent colonies were plated on SD/-ura/-trp/-leu/-his+3 mM 3-AT (3-amimo-1,2,4-triazloe). Large colonies were streaked and subjected to β-Galactosidase colony-lift filter assay. The yeast colonies showing both HIS3 and LacZ reporter gene activation were selected. Library plasmids from these HIS3+ and LacZ+colonies were rescued by transformation of yeast plasmid DNA into KC8 E. coli followed by selection on M9 minimal medium lacking leucine. Rescued library plasmids encoding proteins that interacted with Acheron bait were sequenced and evaluated by BLAST through the National Center for Biotechnology Information Internet site. To confirm the interaction, rescued library plasmid and bait pAS2-1-Acheron were co-transformed into yeast strain Y190 and subjected to the growth on SD/-ura/-trp/-leu/-his+50 mM 3-AT and b-Galactosidase colony-lift filter assay.

GST-Proteins and In Vitro Protein Binding Assays

The CaM kinase II domain of mCASK-C cDNA was amplified by PCR and cloned in frame into SmaI of pGEX-2T and transformed into E. coli BL21 cells. E. coli cultures were induced with 0.4 mM IPTG, and recombinant proteins were affinity-purified from bacterial lysates with glutathione-Sepharose 4B beads (Pharmacia Biotech). For pull-down assays, radiolabeled Acheron and luciferase was produced from pET-25b(+)-Acheron and luciferase controlled plasmid (Promega) by using in vitro TNT Quick Coupled Transcription/Translation System (Promega) with ³⁵S-methionine as the sole source of methionine, following the manufacture's instructions. 5 μl of ³⁵S-methionine-Acheron and ³⁵S-methionine-luciferase were incubated with equal amount of GST or GST-mCASK-C (CaM kinase II domain) bound to glutathione-Sepharose 4B beads, respectively, under constant rocking for 45 minutes in 2 ml of NETN binding buffer. ³⁵S-methionine-luciferase and GST were used as the controls in the binding assays. The Sepharose beads were pelleted and washed extensively with the binding buffer and were analyzed by SDS/PAGE and autoradiography.

Constructs for Deletion Analysis

Acheron deletion mutants were generated by PCR amplification with complementary primers to pAS2-1-Acheron template. The PCR products were digested with SalI and cloned in frame to SalI site of pAS2-1. The generated constructs were named after the region of amino acids they contained. They were pAS2-1-Acheron (14-347), pAS2-1-Acheron (14-372), pAS2-1-Acheron (14-399), pAS2-1-Acheron (14-439) and pAS2-1-Acheron (340-447). The BamHI fragment from pAS2-1-Acheron was cloned in frame to BamHI site of pAS2-1 to form pAS2-1-Acheron (14-205). The BamHI digested pAS2-1-Acheron vector was self-ligated to form pAS2-1-Acheron (205-447). mCASK-C deletion mutants were generated by PCR amplification with complementary primers to pACT2-mCASK-C template. The PCR products were digested with EcOR1 and XhoI and then cloned in frame to pACT2. The generated constructs were pACT2-mCASK-C (1-105), pACT2-mCASK-C (1-280), pACT2-mCASK-C (1-304), pACT2-mCASK-C (1-315), pACT2-mCASK-C (1-339) and pACT2-mCASK-C (350-897).

### Example 1

### Cloning of Acheron

A cDNA library generated from day 18 ISM RNA from Manduca was constructed in the λ ZapII (Stratagene) vector and screened by plus/minus screening to isolate cDNAs that were differentially-expressed (Schwartz et al., Proc. Natl. Acad. Sci. USA 87(17):6594-98 (1990); Schwartz et al., J. Neurobiol. 23:1312-1326 (1992).

To isolate the human Acheron cDNA, a human subtracted hippocampus oligodT and random primed cDNA library constructed in lZAP II vector (Stratagene) was screened using a human hippocampal EST (M79107) that encodes a protein with high sequence identity to Manduca Acheron (described in Results) as a probe. The original library had 2×10⁶ recombinants and was amplified at high density prior to screening. Approximately 5×10⁴ plaques/150 mm plates were plated on E. coli lawns and transferred to nylon filters (Magna Lift, Osmonics). After cross-linking at 80° C. for 2 hours, the membranes were hybridized with the random primed [α-³²P] dCTP-labeled cDNA at high stringency. Positive plaques were identified by autoradiography, re-screened, and cDNA clones recovered within pBluescript vector by in vivo excision.

DNA sequence analysis revealed that the initial recombinants isolated in this screen were truncated at the 5′ end. In order to obtain full-length human Acheron, a modified inverse RT-PCR technique was used to overcome the strong secondary structure due to the high GC content of the 5′-end. Briefly, total RNA was isolated from human RD rhabdomyosarcoma cells and cDNA was generated by reverse transcription using a gene-specific primer (antisense P1: 5′ GTGCCCGCGGCTCGGCTCCTC 3′; SEQ ID NO:18) close to the known 5′end. cDNA synthesis was accomplished using recombinant Tth polymerase (Promega) in the presence of 5% formamide and 1 mM MnCl2 at 52° C. for 10 minutes and then 75° C. for 20 minutes. The resulting cDNA was circularized and amplified using gene-specific primers: P3 (5′ TCCCCGGCGCCCCGAGTCTC 3′; SEQ ID NO:19) and P2 (5′CGGTACCTCAGCCCCGGCTGG 3′; SEQ ID NO:20) both of which are upstream of P1. PCR was performed with a 1:1 mixture of Tth and Taq polymerases (Promega) in the presence of 5% formamide in a hot-start PCR reaction. After an initial denaturation step of 95° C. for 5 minutes, the sample was subjected to 30 cycles of: 1 minute at 95° C., 1 minute at 70° C., 1 minute at 72° C., followed by a final extension step of 7 minutes at 72° C. The PCR product was blunt ended and cloned into pKS (Stratagene) prior to DNA sequencing.

The genomic human Acheron clone was isolated by screening a human PAC library (RPCI1) generated by Ioannou et al. (Nat. Genet. 6(1):84-9 (1994) in the pCYPAC2N vector and obtained from the MRC Human Genome Project Resource Centre (Hinxton, Cambridge, UK). The library was arrayed on seven 22.2×22.2 cm double spotted filters and screened with a ³²P-dCTP radiolabeled probe consisting of 996 bp (PCR amplified human Acheron cDNA region between nucleotides 459-1454). Two positive PACs (304E10 and 261E2) were recovered and shown by sequence analysis to contain the hAcheron gene. The intron-exon boundaries of human Acheron were identified either by direct sequence analysis of PCR amplified fragments from the PAC clones or by using a GenomeWalker kit Clontech) to analyze restriction enzyme digested fragments from these clones.

Full-length hAcheron cDNA was also cloned in-frame in pFLAG-CMV2-neo vector to produce an N-terminal FLAG-tagged hAcheron protein (FLAG—hAch). A slightly N-terminal (1-33 amino acid) truncated hAcheron (tAch) was cloned into pFLAG-CMV2-neo vector to generate a FLAG-tAch fusion protein and then transferred to the pBabe retroviral vector. Both sense and antisense constructs were isolated.

### Example 2

### Generation of Acheron Polyclonal Antibodies

A fragment of hAch cDNA corresponding to the coding region 97-1398 (tAch) was amplified by PCR and subcloned into the expression vector pET-25b(+) (Novagen). The resulting construct encodes a 434 amino acid fused at the C-terminus with a HSV tag and 6×-His residues. The fusion protein was expressed in E. coli BL21(DE3-pLysS) and purified by affinity chromatography on Ni-NTA agarose beads (Qiagen) under native conditions according to the manufacturer's instructions. Polyclonal antisera were raised in rabbits by injection of about 100 μg of gel-purified fusion proteins in complete Freund's adjuvant. Boosting was carried out with subcutaneous injections every two weeks with 100 μg of proteins in incomplete Freund's adjuvant. Serum was collected after the fifth boost and pre-immune serum was collected as control.

### Example 3

### Biological Effects of Acheron Mis-Expression on C₂C₁₂ Cells

Since Acheron was first identified in skeletal muscles, mouse C₂C₁₂ myoblasts, a well-established in vitro model for myogenesis, were used to define the function of hAch at the cellular level. When cultured in trophic factor-rich growth medium (GM), C₂C₁₂ cells rapidly proliferate. When transferred to low serum differentiation medium (DM), C₂C₁₂ chose one of three developmental fates. The majority of cells upregulate MyoD, followed by myogenin expression, cell cycle withdrawal and terminal differentiation into myotubes. As these myoblasts exit the cell cycle, they up-regulate the expression of retinoblastoma protein (Rb) and the cdk (cycle-dependent kinase) inhibitor p21, which serve to enhance both survival and MyoD stability. A second population of myoblasts express Myf5, but not MyoD, and then enter GO without differentiating into myotubes. These cells represent a pool of ‘reserve cells’ with the capacity to self-renew and the capacity to produce differentiation-competent myoblasts when returned to GM. Myf5 is believed to play an important role in the self-renewal capacity of reserve cells. Expression of the anti-apoptotic protein Bcl-2 is restricted to reserve cells and appears to be the predominant survival mechanism for this sub-population. A final group of cells fails to activate any survival programs and undergoes apoptosis.

Western blotting revealed that Acheron is constitutively expressed in both cycling myoblasts and myotubes and localizes predominantly to the cytoplasm despite containing a putative nuclear localization signal. To study the roles of hAch in regulating cell proliferation, differentiation and apoptosis, monoclonal C₂C₁₂ cell lines stably transfected with FLAG-epitope-tagged expression vectors encoding full-length hAch, an N-terminally truncated dominant negative version lacking the first 33 amino acids (tAch), antisense Acheron (As-Ach) or empty vector were generated. The expression of ectopic protein was confirmed by Western blotting with anti-FLAG antibody, while the expression of As-Ach mRNA was verified by RT-PCR analysis. Like the native protein, ectopic hAch also localized predominantly to the cytoplasm.

All of the transfected cell lines displayed comparable levels of cell death when cultured in GM. Following transfer to DM, control cells exhibited the normal increase in cell death that peaked on day 2 and then decreased when differentiation of myotubes was completed between days at 3 and 4 (myosin heavy chain (MHC)-positive multinucleated cells). The hAch cells displayed a 3-5 fold increase in cell death relative to control cells on day 2 following transfer to DM (FIG. 2). Despite excessive cell loss, many surviving cells did fuse and differentiate into myotubes, although few mononucleated cells remained.

In contrast, expression of tAch or As-Ach greatly inhibited both differentiation and cell death in cultured cells incubated in DM. MHC immunostaining revealed that less than 10% cells carried out terminal differentiation. These data suggest that over-expression of hAch increases the level of cell death upon exposure to DM without interfering with the ability of cells to differentiate, while As-Ach and tAch block apoptosis and differentiation and resulted in the retention large numbers of mononucleated reserve cells. When these mononucleated reserve cells were returned to GM, they were able to proliferate and renew the population. Since comparable results were obtained with tAch and As-Ach, tAch may function as a dominant-negative regulator of hAch function. To test this hypothesis, hAch-expressing C₂C₁₂ cells were transfected with vectors encoding FLAG-tAch, As-Ach or nothing, and five monoclonal transfected lines were isolated for each construct. Ectopic expression of either tAch or As-Ach blocked the Ach-induced increased apoptosis and reduced myotube formation, with tAch being more effective than As-Ach, indicating that tAch does function as a dominant-negative regulator of hAch. In addition, the first 33 amino acids of Ach appear to be essential for normal function. While there are no known structural motifs within this region, there are three threonine and one serine residues that are potential phosphorylation sites.

Thus, over-expression of hAch increases the level of cell death upon exposure to DM without interfering with the ability of cells to differentiate, while As-Ach and tAch block apoptosis and differentiation and result in the retention large numbers of mononucleated reserve cells. Furthermore, tAch functions as a dominant-negative regulator of hach, providing a method for inhibiting Acheron activity and thus inhibiting apoptosis and enhancing cell survival.

### Example 4

### Myogenic Pathways Affected by Mis-Expression of Acheron

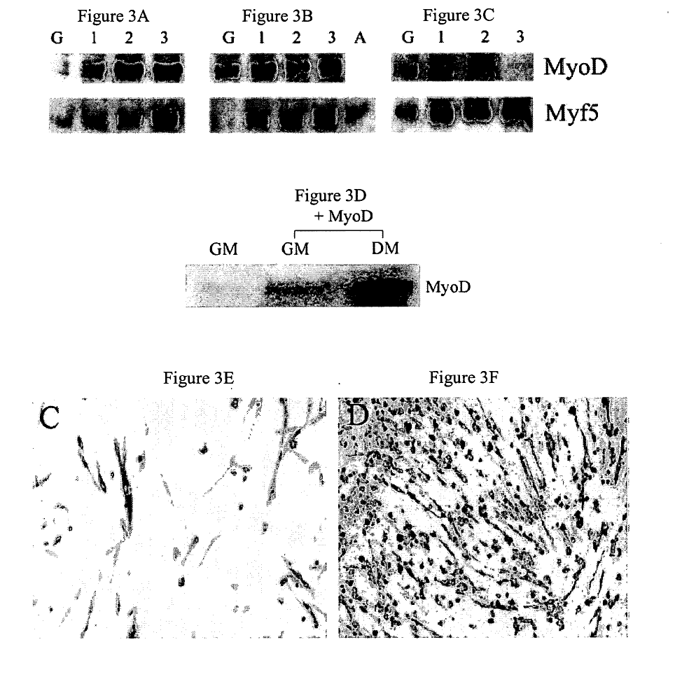

While cycling myoblasts express both MyoD and Myf5, they are restricted to myotubes and reserve cells respectively following transfer to DM. The data described in Example 3 suggest that ectopic hAch pushes C₂C₁₂ cells toward differentiation, while inhibiting hAch using As-Ach and/or tAch pushes cells to the reserve pool. To determine if Ach functions via these helix-loop-helix myogenic transcription factors, the expression of MyoD and Myf5 was examined in four populations of engineered C₂C₁₂ cells (vector control, hAch, t-Ach and As-Ach). Western blot analysis demonstrated that the normal increase in MyoD observed in control and hAch cells following transfer to DM is completely blocked by tAch (FIG. 3A-3C).

Thus it appears that tAch represses MyoD induction and subsequent differentiation. To determine if Acheron functions upstream of MyoD, tAch-expressing C₂C₁₂ cells were stably transfected with a MyoD expression construct, and forced MyoD expression was confirmed by western blotting. Following transfer to DM, these cells produced large numbers of MHC-positive myotubes, suggesting that Acheron is required for normal MyoD expression in myoblasts.

In agreement with phenotypic studies, ectopic hAch blocked Myf5 expression resulting in a 40% decrease in this myogenic factor relative to control cells (FIG. 3B). When the floating apoptotic cells were removed from the hAch-expressing cells, Myf5 was almost undetectable (Lane A; FIG. 3B). These data suggest that Acheron functions to repress Myf5 expression. In agreement with this hypothesis, tAch and As-Ach resulted in a 2-5 fold increase in endogenous Myf5 expression relative to controls. Taken together, these data suggest that Ach is permissive for MyoD expression, but represses Myf5 expression.

### Example 5

### Acheron Induces Apoptosis by Altering the Expression of Bax and Bcl-2

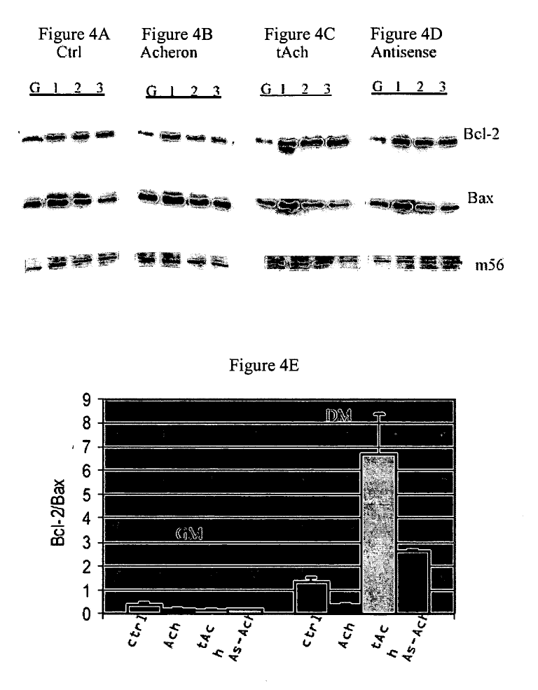

Since hAch blocks Myf5 expression and the survival of mononucleated cells, the expression of Bcl-2, a key survival factor for reserve cells in vitro and satellite cells in vivo was examined. In agreement with published reports, there was a transient increase in Bcl-2 in control cells following transfer to DM (top row, FIG. 4A). The same general pattern of Bcl-2 expression was observed in hAch cells (top row, FIG. 4B), although the absolute levels of expression were well below those seen in control cells. In contrast, by three days after transfer to DM, Bcl-2 levels in tAch cells were four fold higher than control cells and 8-10 time higher than in the hAch-expressing cells (top row, FIG. 4C). As-Ach-transfected cells also displayed enhanced levels of Bcl-2 expression (top row, FIG. 4D).

Since Bcl-2 functions by antagonizing the pro-apoptotic activity of Bax, the western blots shown in the top row of FIG. 4A-D were stripped and reprobed with an anti-Bax monoclonal antibody (middle row, FIG. 4A-D). In control cells, the level of Bax protein paralleled the patterns of apoptosis observed following transfer to DM (middle row, FIG. 4A). Bax increased during the first two days and then fell to basal levels by day 3. While the hAch cells displayed a similar pattern of Bax expression, the absolute levels of the protein were almost twice that seen in control cells (middle row, FIG. 4B). When the floating apoptotic cells were removed from the cultures before western blotting, only about one third of the total Bax protein was present, suggesting that Bax expression was greatest in the apoptotic cells.

In agreement with our observation that blockade of Acheron enhances survival, the levels of Bax proteins were 70% and 30% lower in tAch and As-Ach lines respectively when compared with control cells 2 days after transfer to DM (middle row, FIG. 4C-4D). Since the ratio of Bcl-2-to-Bax is a key determinant in survival, it is worth noting that Bcl-2-to-Bax ratio in tAch and antisense cells was 3-5 fold higher relative to control cells and 10-19 folds higher than in hAch cell (FIG. 4E).

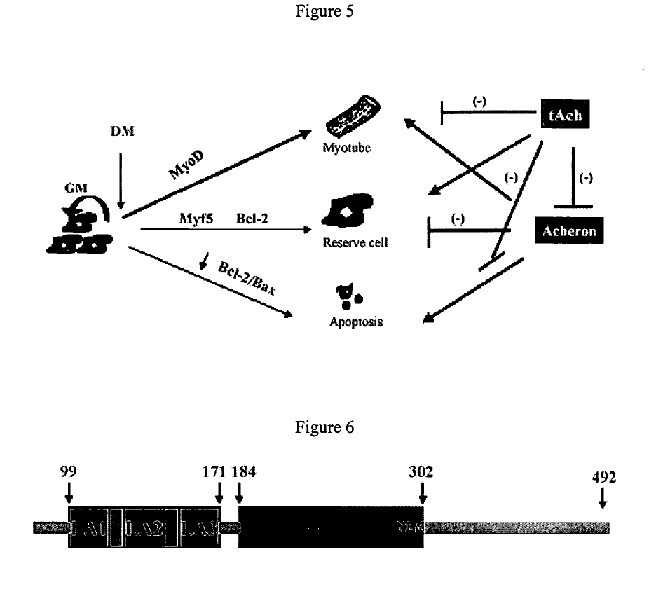

As one theory, not meant to be limiting, the data presented herein suggest that Acheron is a phylogenetically-conserved regulatory protein that plays a key role in the survival and differentiation of muscle cells. In Manduca, Acheron is induced to high levels when the ISMs become committed to die and is blocked when cell death is delayed by hormonal manipulations. Since the biology of Manduca is not conducive to genetic manipulation, mammalian myoblasts were used to study hAch. Data from these experiments suggests the following theoretical model (depicted in FIG. 5): Ach may play a key regulatory role in the differentiative decisions following trophic factor withdrawal by controlling the expression of MyoD, Myf5, Bcl-2 and Bax. Ach is required for MyoD expression and represses Myf5 induction, thus pushing cells towards the differentiation pool. Ach enhances the apoptosis of surplus cells by reducing Bcl-2 while enhancing the expression of Bax. Blockade of Ach enhances the formation of reserve cells by blocking MyoD and enhancing Myf5 expression. The survival of these cells is further insured by the up-regulation Bcl-2 and repression of Bax expression.

Thus, Acheron is a novel phylogenetically-conserved protein that serves to control cellular differentiation and survival, and thus serves as a target for interventions designed to enhance the formation and survival of reserve cells in vitro and satellite cells in vivo.

### Example 6

### Generation of Engineered Myoblasts

CD-1 mice are sacrificed and primary myoblasts prepared from the leg muscles of 2-3 day post-natal pups according to the methods of Rando and Blau (1994) J. Cell Biol. 125(6):1275-87; Rando and Blau (1997) Methods Cell Biol. 52:261-72) to generate cultures that are greater than 98% pure myoblasts. Primary myoblasts are cultured in DMEM supplemented with 20% FBS, 0.5% chick embryo extract and antibiotics. Myoblast purity is determined by staining cultures with an antibody against desmin, a myoblast marker (reference). After enzymatic dissociation of muscles with collagenase (0.2%) and trypsin (0.25%), the cells are cultured in high glucose DMEM at 37° C. for 3 days.

Cultures are expanded, split and transferred to new plates. Acheron activity is inhibited by infection with a retrovirus encoding a dominant negative variant of Acheron, by transfection using lipofection with a plasmid encoding a dominant negative Acheron variant, or by introducing antisense or siRNA targeting Acheron using methods known in the art. In the case of viral infection, each plate is infected with a replication-defective pBabe-puromycin retrovirus encoding a variant of Ach, e.g., an N-terminally truncated Ach (tAch). Retroviruses are packaged in Phoenix cells according to the protocols of the Nolan laboratory, available on the world wide web at stanford.edu/group/nolan/protocols/pro_helper_dep.html and introduced into the primary myoblasts according to the procedures described by Springer and Blau (1997) Somat Cell Mol Genet. 23(3):203-9, who reported greater than 99% infection efficiency. The pBabe constructs use a MMLV LTR (long terminal repeat) to drive high levels of gene expression. Alternatively, adenoviral infection or lipofectamine-mediated transfection with these constructs as plasmids rather than retroviruses is performed.

After infecting or transfecting the primary mouse myoblasts with these constructs, cells are plated in 96 well plates at 40% confluency and then allowed to reach 85% confluency before the growth medium (GM) is replaced with a 2% horse serum/DMEM differentiation medium(DM). Plates are assayed at various times after transfer, including: 0 hrs, 12 hours, 24 hours, 48 hours, 72 hours and 96 hours. One set of plates is stained with calcein-AM and ethidium bromide heterodimer (“Live/Dead” Molecular Probes) and read on a fluorescence plate reader. The calcium AM enters living cells and is de-esterified which traps it in cells and induces fluorescence. The ethidium bromide heterodimer enters dead cells and fluoresces intensely when it intercalates into genomic DNA, therefore, live cells will have green cytoplasm while dead cells will have red nuclei. A number of other assays for apoptosis are known in the art, see, e.g., Schwartz and Osborne, eds. Methods Cell Biol., CELL DEATH. Academic Press 46:459, xv-xviii (1995); Schwartz and Ashwell, eds., Methods in Cell Biology Series, CELL DEATH II. Academic Press, volume 66, pp533 (2001).

Appropriate controls, including empty vectors, full length versions of the Acheron gene, and control sequences that should not impact apoptosis, such as bacterial J-galactosidase, will be performed simultaneously.

### Example 7

### Evaluation of Myoblast Migration and Fusion

Primary mouse myoblasts from CD-1 pups are isolated as described above in Example 6 and plated on collagen-treated plates to facilitate myotube adhesion. After reaching 90% confluency, the cells are incubated in DM for one week with regular media changes to generate large multinucleated myotubes. In separate cultures, infected myoblasts (described above) are grown in GM and then incubated in with PKH26 (which gives red fluorescence) or PKH67 (which gives a green fluorescence) (Torrente et al., Cell Transplant. 9(4):539-49 (2000)). These dyes incorporate into the membrane of cells and are equally distributed to daughter cells following division. Labeled cells are trypsinized and added to myotube cultures described above. Varying concentrations of labeled cells can be evaluated to determine the optimal optical concentration to use to follow the fate of individual cells. Cultures are examined on an inverted fluorescent microscope and photographed at regular intervals to determine the percentage of cells that: 1) survive; 2) adhere to myotubes; 3) migrate along fibers; and 4) fuse with the myotubes. All assays are performed blind to minimize observer bias.

In parallel experiments, the persisting mononucleated cells in the myotube cultures are killed by transiently treating these cultures with Ara-C. After two days of treatment, cultures are extensively washed with saline and then returned to normal DM. Labeled engineered myoblasts are then added to the cultures and monitored visually over time, to determine if there is preferential adhesion or interaction with myotubes versus reserve cells.

To evaluate the potential of transplanted myoblasts to contribute a wild-type dystrophin gene, myotubes generated from C57BL/10ScSnJ mice (Jackson Labs) MDX mouse that carry a point mutation that creates a premature stop codon and a truncated dystrophin protein (Sicinski et al., Science. 244(4912):1578-80 (1989)) will be used as host cells. Both wild type and mutant myoblasts are differentiated into myotubes in vitro. Engineered wild-type and MDX primary myoblasts are labeled with CM-DiI and added to the MDX myotube cultures. As described above, the percentage of cells that migrate along the myotubes and the relative distance traveled and the percentage of cells that fuse with the myotubes are determined. In addition, the myotubes are stained for the expression of dystrophin to determine both the level of expression and its subcellular localization. The functional contribution of dystrophin to these muscle fibers can be evaluated by assays known in the art, e.g., immunohistochemistry, vital dye exclusion, force-tension measurements, or exercise-induced injury. The effect of treatment of myoblasts with growth factors, such as bFGF, fibronectin, TGF-β and hepatocyte growth factor on migration is also evaluated.

### Example 8

### In Vivo Evaluation of Transplant Survival

To evaluate the effect of inhibiting Acheron expression on survival of transplanted cells, CD-1 primary mouse myoblasts are isolated from male donors and prepared as described above. Males are specifically used so that when cells are transplanted into female hosts, the number of ectopic cells can be approximated by performing quantitative PCR with primers directed against Y chromosome-specific sequences. The primary myoblasts are expanded in vitro in growth medium and then infected with the pBabe retroviral vectors, or transfected with the plasmids, as described in Example 6. In these types of transplantation studies, the use of replication defective retroviruses does not appear to induce immunological reactions; Rando and Blau, 1994, supra. If the retroviral vectors do initiate an immune response in host animals, adenovirus-based vectors that lack all expressed viral genes can be used. Non-infected cells and empty vectors serve as controls.

In some experiments, cells are incubated with 0.25 μCi/ml [methyl-14C] thymidine in growth medium 16-24 hours prior to transplantation so that subsequent cell death can be measured in vivo (Skuk article). In separate experiments, myoblasts are stained with PKH26, a fluorescent lineage-marker described above, in order to evaluate cell migration and fusion. In both cases, labeled myoblasts are centrifuged for 5 minutes at 3500 rpm and resuspended in 15% horse serum, centrifuged for 10 minutes at 4000 rpm and resuspended in 10 μl of Hank's balanced salt solution (HBSS) in preparation for injection.

Two to four month old female CD-I mice serve as the hosts for the engineered cells. Both Tibialis anterior (TA) muscles are implanted with the micro-tube technique as previously described (Torrente et al., Cell Transplant. 9(4):539-49 (2000)). Briefly, an IV cannula is used to insert a 0.28 mm diameter polyethylene plastic tube into the muscle parallel to the fibers. The distal end of the tube is sealed and there are 4 small holes placed at 2 mm distances along the length of the tube. Cells are slowly injected from the proximal extremity of the polyethylene micro-tube with a glass micro-pipette (Drummond Scientific Co., Broomall, Pa.) with a 50 μm tip. Cells are injected in a 10 μl volume which satisfies two criteria: first, this volume can be easily injected without causing tissue distortion or swelling; and second, it is 5 μl more than the volume of the micro-tube (5 μl), so that some cells will be expelled immediately from the tube. Control myoblasts that have been labeled but not genetically-engineered will be injected into the contralateral TA muscles.

Muscles are isolated at various times after myoblast transfer and assayed as follows.

1. Survival: At various times after myoblast transfer, host animals are sacrificed and the TA muscles removed. To determine cell survival, genomic DNA is extracted from the muscles and the level of 14C determined. The “zero” time reference is obtained by injecting cells into a deeply anesthetized animal and immediately extracting the DNA. This controls for loss of label during cell transfer, as well as any quenching that make take place in the sample. In combination with histological analysis, loss of radioactivity will be a measure of cell death and subsequent clearance by macrophages and other cells.

2. Proliferation: At various times after myoblast transfer, host animals are sacrificed and the TA muscles removed. Quantitative PCR is performed using Y chromosome-specific primers as previously described (Pugatsch T, Oppenheim A, Slavin S. Improved single-step PCR assay for sex identification post-allogeneic sex-mismatched BMT. Bone Marrow Transplant. 1996 February; 17(2):273-5). Since host cells lack this sequence, the quantity of PCR product should be proportional to the number of copies of the target DNA in the sample and should correlate well with the number of transplanted myoblasts that survive and proliferate. In conjunction with the ¹⁴C-thymidine assays, these PCR assays should give a reasonable measure of both cell death and proliferation in the same samples.

3. Histology: At various times after myoblast transfer, host animals are sacrificed and the TA muscles are removed, frozen and used to generate 10 μm cryostat sections. Propodium iodide is used to label nuclei and ectopic myoblasts will be viewed using florescence microscopy to detect the PKH26. Images are captured for analysis using a Pixera camera and analyzed with NIH Image. Measurements of migration distances are performed at 100× magnification as described in (Torrente et al., Cell Transplant. (4):539-49 (2000). Serial cross sections showing the maximum migration distance in each muscle are used to measure the migration distance from the injection site depicted by the micro-tube. Concentric equidistant (50 μm) circles are superimposed on the photograph of the selected muscle cross-section, and migration is measured from the bold circle corresponding to the external surface of the micro-tube up to the farthest located group of fluorescent cells giving the maximum migration range. The significance of the differences is evaluated using an analysis of variance (ANOVA) on a Stat View 512 software (Brain Power, Calabasas, Ca) with a level of p<0.05 being considered significant. The person performing the assays is blind as to the molecular-genetic manipulations performed on the test animals.

### Example 9

### Evaluation of the Ability of Transplanted Cells to Facilitate Repair

Primary myoblasts are isolated as described above (Rando and Blau, 1997, supra) from C57BL/10ScSnJ mice (Jackson Labs) and engineered with the constructs described herein to alter Acheron activity. Engineered myoblasts are then transferred into wild-type and C57BL/10ScSnmdx/J mice. These animals are completely histocompatible (Vilquin et al., J. Cell Biol. 131(4):975-88 (1995), so issues related to rejection are minimized. Interestingly, these animals do generate anti-dystrophin antisera in their blood (Vilquin et al., 1995, supra), but this does not lead to complement fixation or rejection. In fact, pretreatment of mdx mice with a dystrophin peptide tolerizes the animals and blocks this response. As a control, the contralateral TA muscle receives labeled wild-type myoblasts.

At 5 days and three weeks after myoblast injection, muscles are examined for both dystrophin immunoreactivity and PKH26 fluorescence. The presence of dystrophin in the mdx muscle allows the evaluation of the functional contributions made by the transplanted cells, since no endogenous dystrophin should be expressed in these animals. (It should be noted that some mdx mice do express dystrophin-reactive peptides, so proper controls and sample sizes are performed (Hoffman et al., J Neurol Sci. 99(1):9-25 1990).

In addition to these anatomical assays, the ability of Acheron inhibition to enhance myoblast survival and provide physical protection to mdx muscles is determined. While the mdx phenotype is not as severe as that seen in patients with DMD, these animals do display muscle apoptosis (Sandri et al. Neurosci. Lett. 252(2):123-6 (1998) and secondary fiber necrosis when they are forced to walk on a treadmill (Brussee et al. Neuromuscul. Disord. 7(8):487-92 (1997); Vilquin et al. Muscle Nerve. 21(5):567-76 (1998). To determine if the Acheron-engineered myoblasts contribute to enhanced fiber survival and use, animals are walked on a motorized treadmill at a −15 degrees slope at 10 m/min (Brussee et al., 1997, supra). Apoptosis is detected by TUNEL and/or anti-caspase-3 staining. Necrosis is evaluated by injecting animals with Evans blue 24 hours before sacrifice, to reveal breaches of sarcolemmal integrity by uptake of this vital dye. The levels of TUNEL and Evans blue staining is determined within each subject by comparing the test and contra-lateral muscles.

### Example 10

### Identification of mCASK-C by Interaction with Acheron by Yeast Two-hybrid Screening

To identify proteins that interact with Acheron, the entire known mouse Acheron coding region (lacking only a small part of the N-terminus) was cloned in frame to C terminus of the DNA-binding domain of GAL4 to create the bait in the plasmid pAS2-1. The bait pAS2-1-Acheron was transformed into yeast strain CG1945 carrying two reporter genes, HIS3 and LacZ. The expression of fusion bait protein was checked by Western blotting with the antibody against Acheron. The bait plasmid did not activate the expression of the two reporter genes by itself. To identify the potential protein interaction partners for Acheron, mouse 17-day embryo cDNA library (Clontech) was amplified and transformed into yeast strain CG1945 containing bait pAS2-1-Acheron. About 4.8×10⁶ transformants were plated, and two clones were confirmed positive for both HIS3 and lacZ expression. The two prey plasmids were rescued and isolated. After sequence analysis and BLAST search, it was determined that one of the prey plasmids encoded a full-length murine protein belonging to the CAMGUK family (Genomics 53, 29-41 1998), which contained the combination of an N-terminal CaM kinase II domain and a C-terminal MAGUK domains. This protein shares very high identity with human CASK at both the DNA and protein levels, and so was named mCASK-C. The other prey plasmid encodes c-terminus of Ariadnen, containing part of the second ring finger.

To further confirm the interaction between mouse Acheron and mCASK-C, another two-hybrid assay was conducted in yeast strain Y190. Isolated prey plasmid pACT2-mCASK-C, bait plasmid pAS2-1-Acheron, vector plasmid pAS2-1 and pACT2 were co-transformed into yeast strain Y190 by combination. The transformants were grown on SD/-ura/-trp/-leu/-his+50 mM 3-AT and P-Galactosidase colony-lift filter assay. Only yeast strain Y190 carrying both pAS2-1-Acheron and pACT2-mCASK-C was positive for HIS3 and LacZ expression. Transformants with all other combinations of plasmids did not show positive expression. This indicated that the GAL4-BD-Acheron fusion protein did not interact with GAL4-AD protein, and that the GAL4-AD-mCASK-C fusion protein did not interact with GAL4-BD protein. The LacZ and HIS3 reporter genes appear to be activated only when GAL4-BD-Acheron and GAL4-AD-mCASK-C fusion proteins were expressed in yeast cells concurrently.

Thus, Acheron interacts specifically with mCASK-C.

### Example 11

### Cloning of Murine CASK-C

To further characterize mCASK-C, the rescued library plasmids encoding proteins that interacted with the Acheron bait as described above were sequenced and evaluated by BLAST. It was determined that one encoded a full-length putative CAMGUKs protein, later termed mCASK-C. The coding region of mCASK-C spans 2694 nucleotides (SEQ ID NO:9) and encodes a protein of 897 amino acids (SEQ ID NO:10). It shares 95% identity at DNA level and 99.6% identity at protein level with human CASK (“hCASK”), only three amino acids difference (Pro395 against Leu395, Ser777 against Leu777 and Val852 against Ile852) between them. Like hCASK, mouse CASK-B and rat CASK, the putative mCASK-C is composed of a series of protein domains: the N-terminal CaM Kinase II domain (amino acids 1-339), which contains protein kinase subdomain (amino acids 12-276) and calmodulin binding subdomain (amino acids 305-315), the C-terminal PDZ domain (amino acids 483-558), SH3 domain (amino acids 587-652) and GUK domain (amino acids 710-831) forming core MAGUK motifs. This combination of N-terminal CaM kinase II domain and C-terminal MAGUK domains has been recently described as a new emerging protein family CAMGUKs (Genomics 53, 29-41 1998). The CaM Kinase II domain and PDZ domain of mCASK-C, hCASK, mouse CASK-B, and rat CASK are identical except one amino acid difference between mouse CASK-B and others (L298 versus F298). The SH3 domain and GUK domains of these four proteins are highly conserved. However, compared to mCASK-B, mCASK-C shows a deletion of 6 amino acids (amino acids 340-345) just downstream CaM Kinase II domain and a deletion of 23 amino acids (amino acids 580-602) downstream PDZ domain. The deletion of amino acids 340-345 was described as an alternatively used exon in all isolates of mCASK-A and mCASK-B.

### Example 12

### In Vitro Binding Assays

To further confirm the physical interaction between Acheron and mCASK-C, an in vitro protein binding assay was performed. 35 S-labeled proteins were first synthesized by in vitro transcription and translation, and then were incubated with GST or GST-mCASK-C (CaM kinase II domain from amino acid 1 to 339) immobilized on glutathione-Sepharose 4B beads. The beads were pelleted and washed extensively and the bound protein complex was resolved by SDS/PAGE and detected by autoradiography. Acheron was found to bind with GST-mCASK-C but not with GST, and GST-mCASK-C did not bind with control protein luciferase.

These findings confirm that Acheron physically associates with mCASK-C in vitro.

### Example 13

### Determination of the Regions of Interaction Between Acheron and mCASK-C

To determine the responsible interaction region of Acheron with mCASK-C, a series of deletion mutants from Acheron were generated and fused in frame with DNA-binding domain of Gal4 in pAS2-1. Each generated construct was co-transformed into yeast strain Y190 with pACT2-mCASK-C. The transformants were evaluated by a P-Galactosidase colony-lift filter assay.

To determine the responsible interaction region of mCASK-C with Acheron, a series of deletion mutants from mCASK-C were generated and fused in frame with DNA-activation domain of Gal4 in pACT2. Each generated construct was co-transformed into yeast strain Y190 with pAS2-1-Acheron. The transformants were evaluated by a P-Galactosidase colony-lift filter assay.

By deletion analysis, the carboxy-terminal region of Acheron (amino acids 340-439) was found to be necessary and sufficient for physical interaction with part of the CaM Kinase II domain (amino acids 1-304) of mCASK-C. The calmodulin binding subdomain in the CaM Kinase II domain is not necessary for association between Acheron and mCASK-C. Since the CaM Kinase II domain shares very high identity among CASK proteins, Acheron may interact with other CASK proteins.

CASK contains multiple protein-binding domains that allow them to assemble specific multi-protein complexes in particular regions of the cell (Cell 93, 495-498:1998; Curr. Biol. 6, 382-384:1996). CASK protein contains a putative CaM Kinase II domain, and the carboxy-terminal of Acheron contains putative motifs that may act as kinase substrates. Thus, it is reasonable to predict that mCASK-C may phosphorylate Acheron. As one theory, not meant to be limiting, Acheron may act as a carrier for nuclear translocation of CASK, since Acheron contains a nucleus localization sequence and CASK is a membrane-associated protein.

### Example 14

### The Effect of Acheron on Metastatic Potential

CHO (hamster ovary fibroblasts) with normal expression levels of EGFR and A431 (human epidermoid carcinoma cells) with high levels of EGFR expression were treated with 100 ng/ml EGF for 5 minutes, 30 minutes and 2 hours. In the untreated CHO cells, Acheron staining was cytoplasmic, diffuse and weak, but after 2 hours of treatment, the cells showed intense nuclear Acheron staining. In contrast, the A431 cells showed very intense nuclear Acheron staining regardless of the treatment. The primary antibody was generated as described herein; the secondary antibody was fluorescein conjugated goat anti rabbit.

Two rhabdomyosarcoma cell lines, RH—I and RH-39 showed very different patterns of Acheron expression. Rh-i cells have nuclear staining only, while Rh-39 cells show cytoplasmic and nuclear expression. Cells were cultured in DMEM with 15% FBS. Staining was carried out by ICC using the Vector staining kit and DAB as chromogen, polyclonal antibodies against the synthetic peptide and the N-terminal truncated form, dilution 1:100-1:500.

Thus, Acheron is translocated to the nucleus in response to the addition of trophic factors in EGF-sensitive CHO cells, and is located in the nucleus in a number of cell lines; this pattern of translocation/localization to the nucleus correlates with greater invasiveness and oncogenic potential.

### Example 15

### Methods of Inhibiting Acheron Expression or Activity

cDNA constructs that express truncated (dominant-negative) Acheron from the B-myb promoter have been generated. The B-myb regulatory sequence is dramatically induced during the G1/S phase of the cell cycle, and then transcriptionally repressed during GO (Joaquin M, Watson R J. (2003) Cell cycle regulation by the B-Myb transcription factor. Cell Mol Life Sci. 60:2389-401.). This means that while cells are cycling, dominant-negative Acheron will be expressed and can influence survival.

cDNA constructs using the pGLHB-myb-luciferase promoter reporter construct (Lam et al., Gene 160(2):277-81 (1995)), have been generated that include the luciferase cDNA and a Tinkered Acheron gene. Two constructs were generated: full length Acheron (pB-myb-FL-44a) and a truncated dominant-negative version (pB-myb-TR-44a).

These constructs are introduced into primary myoblasts with Nucelofectin and populations of transfected cells selected with three days incubation in puromycin. Cells are cultured for several days in growth medium (GM) before transfer to differentiation medium (DM) and the subsequently assayed. The expression of reporter genes from the B-myb promoter can be monitored to verify that ectopic expression is substantially reduced when the cells are transferred to DM, which is anticipated based on promoter-reporter assays. If expression continues well after transfer to DM, other promoters such as Cdk2 and tetracycline are used. Cells are then assayed for their ability to survive in the absence of trophic support and for the capacity to incorporate into muscle fibers in vivo following transplantation.

When the cells are transplanted into a trophic-deficient environment in vivo, expression will be repressed. As the ectopic protein levels decrease, the presumptive block to differentiation resulting from dominant-negative Acheron is removed. Preliminary studies demonstrated that a B-myb-promoter-luciferase-reporter construct was induced better than ten fold in cycling C₂C₁₂ myoblasts. When cells were grown to confluency in growth medium or transferred to differentiation medium, luciferase activity was reduced to the level of a promoterless luciferase control reporter construct.

### Example 16

### Acheron Expression Profiling

Mouse satellite cell tissue culture cell lines that stably express either Acheron or truncated Acheron were created, and gene expression profiling was performed.

RNA Hybridization

Total RNA was isolated from mouse satellite cell tissue culture cell lines stably expressing either Acheron or truncated Acheron. The integrity of the purified total RNA was confirmed using the Agilent 2100 Bioanalyzer (Agilent Technologies, Palo Alto, Calif.).

Hybridization samples were prepared according to the Affymetrix GeneChip Expression Analysis Manual (Affymetrix, Santa Clara, Calif.). Briefly, 10 μg of total RNA was used to generate first-strand cDNA. After second-strand synthesis, biotinylated and amplified RNA were purified using GeneChip Sample Cleanup Module (Affymetrix, Santa Clara, Calif.) and quantitated by a spectrophotometer. Biotinylated cRNA samples were then hybridized to Affymetrix mouse MOE 430A arrays. These arrays contain probe sets for 22690 transcripts and EST clones. After hybridization, the microarrays were washed, scanned, and analyzed with the GENECHIP software (Affymetrix, Santa Clara, Calif.).

Data Checking and Analysis

1) Data checking: 24.CEL files generated by the Affymetrix Microarray Suite (MAS) version 5.0 were checked using 2 plotting techniques-boxplot and histogram. Three chips were identified to be different from their corresponding replicates in the same group by both plots. They are PGM8, FDM16, and PDM24. Therefore, these three samples were eliminated from further analyses.

2) Analysis: The PM (perfect match) probe intensities were corrected by RMA, normalized by quantile normalization, and summarized using medianpolish (all of these are conveniently implemented in the RMA method in Affy package of Bioconductor, available on their website). The comparison of global gene expression profiles was done using two sample t-test assuming normal distributions and unequal variances between the two groups. The differentially expressed genes were selected to be significant according to False Discovery Rate (FDR)<0.05.

Results:

All the genes that were called significantly different (with FDR<0.05) in any of the 7 comparisons are shown in Table 4. Comparisons were named by the samples involved in the comparisons. F stands for Full length Acheron, P stands for truncated Acheron, C stands for control Babe, GM stands for growth condition, and DM stands for differentiation condition. Each comparison has 6 output columns. For example, FGMvsCGM stands for the comparison between full-length Acheron in GM and control Babe in growth medium (genes normally changed by over-expression of Acheron in normal medium); PGMvsCGM=truncated dominant-negative Acheron versus control in growth medium (genes normally changed by over-expression of Acheron in normal medium); FDMvsCDM=Acheron versus control in differentiation medium (genes normally changed by over-expression of Acheron under conditions that probably more closely resemble the normal tissues); PDMvsCDM=truncated dominant-negative Acheron versus control in differentiation medium (genes normally changed by over-expression of Acheron under conditions that probably more closely resemble the normal tissues); FDMvsFGM=Acheron in differentiation medium versus Acheron in growth medium (there are some dramatic changes in muscle-specific genes and also fat genes such as Genomic organization, chromosomal localization and adipocytic expression of the murine gene for CORS-26 which is dramatically repressed by truncated Acheron and induced by full-length); PDMvsPGM=truncated Acheron in differentiation medium versus truncated Acheron in growth medium.

The results below show fold change (the ratio of mean FGM original expression level to mean CGM original expression level), and whether it is Increase (i.e., fold change >1) or Decrease (i.e., fold change <1), respectively.

TABLE 4
Acheron Expression Profiling Results

| | | FGMvs | PGMvs | FDMvs | PDMvs | FDMvs | PDMvs |
|---|---|---|---|---|---|---|---|
| gene name | gene | CGM | CGM | CDM | CDM | FGM | PGM |
| Nmyc1 | neuroblastoma myc- | 0.041 | 1.113 | 0.075 | 0.759 | 1.005 | 0.811 |
| | related oncogene | | | | | | |
| Sfrp2 | secreted frizzled- | 0.103 | 0.1 | 0.051 | 0.045 | 1.057 | 0.952 |
| | related sequence | | | | | | |
| | protein 2 | | | | | | |
| Tnnc2 | troponin C2, fast | 3.064 | 0.875 | 0.987 | 0.072 | 28.343 | 2.941 |
| Mylpf | myosin light chain, | 5.236 | 0.662 | 0.725 | 0.075 | 15.58 | 5.954 |
| | phosphorylatable, fast | | | | | | |
| | skeletal muscle | | | | | | |
| Tncc | troponin C, | 2.714 | 0.615 | 0.832 | 0.076 | 17.916 | 3.211 |
| | cardiac/slow skeletal | | | | | | |
| Corcs-pending | | 2.17 | 0.644 | 1.086 | 0.077 | 14.183 | 1.467 |
| Col6a2 | procollagen, type VI, | 0.163 | 0.199 | 0.148 | 0.085 | 2.765 | 1.115 |
| | alpha 2 | | | | | | |
| Myog | myogenin | 5.571 | 0.896 | 0.777 | 0.095 | 6.988 | 2.662 |
| Myl1 | myosin, light | 2.552 | 0.697 | 0.852 | 0.096 | 9.594 | 1.918 |
| | polypeptide 1, alkali; | | | | | | |
| | atrial, embryonic | | | | | | |
| Col6a2 | procollagen, type VI, | 0.283 | 0.269 | 0.168 | 0.111 | 1.919 | 1.168 |
| | alpha 2 | | | | | | |
| Acta1 | actin, alpha 1, | 5.274 | 1.111 | 0.78 | 0.117 | 10.195 | 3.937 |
| | skeletal muscle | | | | | | |
| 1110002H13Rik | | 1.907 | 0.799 | 1.071 | 0.119 | 13.275 | 1.673 |
| Tnni1 | troponin I, skeletal, | 2.157 | 0.823 | 1.272 | 0.125 | 16.637 | 1.971 |
| | slow 1 | | | | | | |
| Chrna1 | cholinergic receptor, | 1.788 | 0.256 | 0.815 | 0.153 | 1.325 | 0.992 |
| | nicotinic, alpha | | | | | | |
| | polypeptide 1 (muscle) | | | | | | |
| Mybph | myosin binding protein H | 1.815 | 0.873 | 1.887 | 0.179 | 24.331 | 2.215 |
| Cdh15 | cadherin 15 | 2.486 | 0.575 | 0.737 | 0.185 | 1.192 | 0.837 |
| Islr | immunoglobulin | 2.764 | 0.394 | 0.338 | 0.194 | 0.583 | 1.961 |
| | superfamily containing | | | | | | |
| | leucine-rich repeat | | | | | | |
| Tubb3 | tubulin, beta 3 | 2.425 | 0.226 | 1.01 | 0.197 | 0.75 | 0.937 |
| Myh3 | myosin, heavy | 2.534 | 0.917 | 2.149 | 0.2 | 41.381 | 4.991 |
| | polypeptide 3, | | | | | | |
| | skeletal muscle, | | | | | | |
| | embryonic | | | | | | |
| 1110027O12Rik | | 3.244 | 0.77 | 0.988 | 0.203 | 2.287 | 1.125 |
| Nsg1 | neuron specific gene | 0.318 | 0.268 | 0.265 | 0.213 | 1.205 | 1.034 |
| | family member 1 | | | | | | |
| Pkia | protein kinase | 2.304 | 0.7 | 0.748 | 0.217 | 1.844 | 1.145 |
| | inhibitor, alpha | | | | | | |
| Cxcl12 | chemokine (C-X-C | 0.2 | 0.468 | 0.198 | 0.219 | 3.605 | 1.723 |
| | motif) ligand 12 | | | | | | |
| Bicc1 | bicaudal C homolog 1 | 0.282 | 0.249 | 0.268 | 0.228 | 1.229 | 1.148 |
| | (Drosophila) | | | | | | |
| Lmcd1 | LIM and cysteine-rich | 0.164 | 0.214 | 0.21 | 0.24 | 1.462 | 1.322 |
| | domains 1 | | | | | | |
| Sorcs2-pnding | | 1.936 | 0.463 | 0.653 | 0.24 | 1.221 | 1.362 |
| Npnt | nephronectin | 1.94 | 0.262 | 1.335 | 0.244 | 1.518 | 1.148 |
| Bgn | biglycan (bone) | 0.057 | 0.096 | 0.264 | 0.246 | 6.827 | 3.583 |
| Igf2 | insulin-like growth | 1.892 | 0.977 | 0.808 | 0.261 | 3.449 | 1.528 |
| | factor 2 | | | | | | |
| Pkia | protein kinase | 2.06 | 0.784 | 0.73 | 0.276 | 1.598 | 1.102 |
| | inhibitor, alpha | | | | | | |
| Dapk2 | death-associated | 2.612 | 0.769 | 1.028 | 0.289 | 2.175 | 1.381 |
| | kinase 2 | | | | | | |
| Npnt | nephronectin | 2.327 | 0.29 | 1.547 | 0.297 | 1.749 | 1.534 |
| Lmyc1 | lung carcinoma myc | 2.22 | 0.649 | 0.692 | 0.3 | 1.152 | 1.34 |
| | related oncogene 1 | | | | | | |
| Ptn | pleiotrophin | 0.26 | 0.238 | 0.3 | 0.312 | 1.166 | 1.372 |
| C630002M10Rik | | 0.168 | 0.249 | 0.247 | 0.313 | 1.285 | 1.173 |
| Car3 | carbonic anhydrase 3 | 1.899 | 1.277 | 0.362 | 0.323 | 0.482 | 0.613 |
| Col6a1 | procollagen, type VI, | 0.161 | 0.374 | 0.203 | 0.325 | 2.586 | 2.112 |
| | alpha 1 | | | | | | |
| Tnnt1 | troponin T1, skeletal, | 2.473 | 2.917 | 1.362 | 0.329 | 5.984 | 0.759 |
| | slow | | | | | | |
| Bgn | biglycan | 0.098 | 0.156 | 0.346 | 0.339 | 5.024 | 2.998 |
| 6330406I15Rik | | 0.26 | 0.329 | 0.211 | 0.339 | 2.14 | 3.155 |
| My14 | myosin, light | 2.268 | 0.896 | 3.325 | 0.342 | 33.149 | 3.944 |
| | polypeptide 4, alkali; | | | | | | |
| | atrial, embryonic | | | | | | |
| 2810002E22Rik | | 0.199 | 0.426 | 0.257 | 0.359 | 2.402 | 1.727 |
| Chrnb1 | cholinergic receptor, | 2.075 | 0.716 | 1.219 | 0.364 | 1.931 | 1.134 |
| | nicotinic, beta | | | | | | |
| | polypeptide 1 (muscle) | | | | | | |
| Bgn | biglycan | 0.096 | 0.179 | 0.389 | 0.369 | 5.864 | 2.929 |
| Cd80 | CD80 antigen | 1.837 | 0.534 | 1.155 | 0.39 | 1.296 | 1.055 |
| Gap43 | growth associated | 0.262 | 0.204 | 0.52 | 0.416 | 0.823 | 0.775 |
| | protein 43 | | | | | | |
| Cmah | cytidine monophospho- | 4.18 | 0.839 | 2.626 | 0.421 | 2.022 | 0.913 |
| | N-acetylneuraminic | | | | | | |
| | acid hydroxylase | | | | | | |
| Wnt10a | wingless related MMTV | 0.349 | 0.401 | 0.788 | 0.433 | 2.56 | 0.981 |
| | integration site 10a | | | | | | |
| Ank1 | ankyrin 1, erythroid | 2.065 | 0.663 | 1.43 | 0.446 | 1.682 | 1.216 |
| Aebp1 | AE binding protein 1 | 0.133 | 0.399 | 0.236 | 0.478 | 1.827 | 1.564 |
| Crlf1 | cytokine receptor-like | 0.204 | 0.53 | 0.196 | 0.481 | 1.066 | 1.388 |
| | factor 1 | | | | | | |
| Nef3 | neurofilament 3, | 3.917 | 0.4 | 1.623 | 0.516 | 0.378 | 0.85 |
| | medium | | | | | | |
| Gsta2 | glutathione S- | 0.215 | 0.076 | 0.973 | 0.517 | 0.686 | 0.875 |
| | transferase, alpha 2 | | | | | | |
| | (Yc2) | | | | | | |
| Figf | c-fos induced growth | 0.233 | 0.271 | 0.505 | 0.517 | 1.056 | 0.921 |
| | factor | | | | | | |
| Aebp1 | AE binding protein 1 | 0.138 | 0.456 | 0.23 | 0.541 | 1.919 | 1.826 |
| Stc | stanniocalcin 1 | 0.172 | 0.164 | 0.679 | 0.569 | 1.182 | 0.972 |
| Cmah | cytidine monophospho- | 2.314 | 0.925 | 2.182 | 0.6 | 1.76 | 0.825 |
| | N-acetylneuraminic | | | | | | |
| | acid hydroxylase | | | | | | |
| Sod3 | superoxide dismutase | 0.243 | 0.704 | 0.405 | 0.636 | 1.516 | 0.95 |
| | 3, extracellular | | | | | | |
| Adss | adenylosuccinate | 0.231 | 0.745 | 0.841 | 0.663 | 2.2 | 0.483 |
| | synthetase, muscle | | | | | | |
| A1BG | alpha-1-B glycoprotein | 0.293 | 0.662 | 0.771 | 0.663 | 1.882 | 0.688 |
| Npy1r | neuropeptide Y | 0.335 | 0.407 | 0.65 | 0.775 | 0.995 | 1.024 |
| | receptor Y1 | | | | | | |
| Gzme | granzyme E | 8.715 | 0.949 | 3.836 | 0.823 | 0.817 | 0.976 |
| Ugtla1 | UDP- | 0.238 | 0.438 | 0.358 | 0.824 | 0.802 | 1.307 |
| | glucuronosyltransferase | | | | | | |
| | 1 family, member 2 | | | | | | |
| Krt1-19 | keratin complex 1, | 1.943 | 0.835 | 1.436 | 0.872 | 0.464 | 0.553 |
| | acidic, gene 19 | | | | | | |
| Vdr | vitamin D receptor | 0.347 | 0.682 | 0.563 | 0.884 | 1.17 | 1.068 |
| Mcpt8 | mast cell protease 8 | 3.68 | 0.907 | 1.546 | 0.9 | 0.52 | 1.027 |
| Gzmd | granzyme D | 9.764 | 0.882 | 3.456 | 0.917 | 0.544 | 1.023 |
| Gzmd | granzyme D | 8.953 | 0.919 | 3.054 | 0.941 | 0.479 | 0.975 |
| Pdgfrb | platelet derived | 0.283 | 0.824 | 0.276 | 0.942 | 1.773 | 3.191 |
| | growth factor | | | | | | |
| | receptor, beta | | | | | | |
| | polypeptide | | | | | | |
| Cxc15 | chemokine (C-X-C | 0.07 | 0.455 | 0.343 | 1.026 | 0.557 | 0.358 |
| | motif) ligand 5 | | | | | | |
| Glrx1 | glutaredoxin 1 | 0.329 | 0.766 | 0.498 | 1.052 | 0.933 | 1.052 |
| | (thioltransferase) | | | | | | |
| Trfr | transferrin receptor | 2.408 | 1.776 | 2.128 | 1.081 | 1.27 | 0.694 |
| Mgst2 | microsomal glutathione | 0.294 | 0.648 | 0.863 | 1.094 | 0.888 | 0.54 |
| | S-transferase 2 | | | | | | |
| Pdgfrb | platelet derived | 0.188 | 0.935 | 0.237 | 1.099 | 2.252 | 3.477 |
| | growth factor | | | | | | |
| | receptor, beta | | | | | | |
| | polypeptide | | | | | | |
| Cpne2 | copine II | 0.331 | 0.654 | 1.118 | 1.105 | 1.615 | 0.813 |
| Pcdhb17 | protocadherin beta 17 | 1.924 | 1.382 | 1.305 | 1.107 | 2.245 | 2.475 |
| AW060714 | | 3.004 | 1.679 | 1.307 | 1.111 | 1.182 | 1.674 |
| Tagln | transgelin | 2.576 | 0.793 | 1.338 | 1.212 | 2.114 | 6.082 |
| Osmr | oncostatin M receptor | 0.184 | 0.96 | 0.389 | 1.263 | 1.582 | 1.447 |
| Thbd | thrombomodulin | 0.21 | 0.616 | 0.487 | 1.263 | 0.898 | 1.081 |
| Cxcl1 | chemokine (C-X-C | 0.25 | 0.764 | 0.781 | 1.303 | 0.97 | 0.622 |
| | motif) ligand 1 | | | | | | |
| Igfbp4 | insulin-like growth | 0.332 | 1.538 | 0.469 | 1.315 | 3.221 | 2.719 |
| | factor binding protein 4 | | | | | | |
| Glipr1 | GLI pathogenesis- | 2.27 | 1.121 | 1.643 | 1.352 | 0.527 | 0.847 |
| | related 1 (glioma) | | | | | | |
| Aqp5 | aquaporin 5 | 0.238 | 1.091 | 0.706 | 1.551 | 1.42 | 0.9 |
| MGC36851 | | 2.163 | 1.997 | 1.236 | 1.857 | 0.872 | 1.601 |
| 1110002J03Rik | | 2.089 | 2.049 | 1.28 | 1.894 | 1.029 | 1.749 |
| Actg2 | actin, gamma 2, smooth | 2.789 | 1.656 | 2.519 | 1.911 | 1.451 | 1.691 |
| | muscle, enteric | | | | | | |
| Khdrbs3 | KH domain containing, | 0.23 | 1.931 | 0.426 | 2.029 | 0.952 | 0.893 |
| | RNA binding, signal | | | | | | |
| | transduction | | | | | | |
| | associated 3 | | | | | | |
| Tsrc1 | thrombospondin repeat | 0.32 | 1.232 | 0.922 | 2.189 | 1.371 | 1.134 |
| | containing 1 | | | | | | |
| Serpine2 | serine (or cysteine) | 0.204 | 1.134 | 0.345 | 2.306 | 0.646 | 1.416 |
| | proteinase inhibitor, | | | | | | |
| | clade E, member 2 | | | | | | |
| Nap1l2 | nucleosome assembly | 1.876 | 2.496 | 1.862 | 2.326 | 0.779 | 0.773 |
| | protein 1-like 2 | | | | | | |
| Robo1 | roundabout homolog 1 | 1.893 | 2.309 | 1.192 | 2.359 | 0.636 | 1.291 |
| | (Drosophila) | | | | | | |
| F3 | coagulation factor III | 0.326 | 0.868 | 0.771 | 3.051 | 1.215 | 2.832 |
| Fgf7 | fibroblast growth | 0.185 | 1.669 | 1.077 | 3.917 | 1.418 | 0.881 |
| | factor 7 | | | | | | |
| Cd24a | CD24a antigen | 0.188 | 5.469 | 0.823 | 5.209 | 0.646 | 0.24 |
| Cck | cholecystokinin | 0.162 | 1.321 | 0.725 | 5.362 | 0.21 | 0.354 |
| Cdh10 | cadherin 10 | 3.943 | 3.5 | 1.768 | 6.092 | 0.302 | 1.743 |
| Cd24a | CD24a antigen | 0.159 | 5.633 | 0.874 | 7.062 | 0.617 | 0.275 |
| Atp1b1 | ATPase, Na+/K+ | 0.292 | 4.543 | 3.244 | 7.41 | 2.139 | 0.394 |
| | transporting, beta 1 | | | | | | |
| | polypeptide | | | | | | |
| Gabra1 | gamma-aminobutyric | 2.014 | 7.729 | 2.637 | 21.079 | 0.502 | 1.983 |
| | acid (GABA-A) | | | | | | |
| | receptor, subunit | | | | | | |
| | alpha 1 | | | | | | |

Thus, the methods described herein include the use of Acheron polypeptide, nucleic acids, and fragments thereof to modulate the expression or activity of one of these genes.

These results suggest that Acheron influences several key biological processes, including cancer, cell differentiation and cell death. Some key observations are presented here.

1) As described herein, the tissue with the highest levels of Acheron expression is the nervous system. In situ hybridization suggests that in the developing brain, the highest levels are in post-mitotic neurons. In this regard, it is interesting that the gene with the greatest fold change in expression in response to Acheron is the cancer gene neuroblastoma myc-related oncogene. This suggests that Acheron may be relevant to brain cancers and defects in brain development, including, but not limited to, neuroblastoma, glioblastoma, Medulloblastoma, Meningioma, Downs Syndrome, and autism. The last two diseases may be related to the cell division and death of neurons under the influence of Acheron.

2) Acheron serves to repress the expression of a number of bone-associated genes including biglycan, stanniocalcin 1, and procollagen, type VI, alpha 2. This suggests that targeting Acheron may be relevant to diseases of bone including, but not limited to, osteoarthritis, osteoporosis, bone repair, metastasis to bone, and osteosarcoma.

3) While Acheron is expressed in almost all tissues, it is largely absent from normal and malignant lymphoid tissues including: bone marrow, thymus, spleen, and lymphomas. This observation may suggest that Acheron functions as a negative regulator of differentiation lymphoid lineages and therefore may play a role in leukemia and lymphoma and related diseases. Given that Acheron serves to enhance and repress the expression of the basic helix-loop-helix (bHLH) transcription factors MyoD and Myf5 respectively in C₂C₁₂ cells, it is possible that it could serve a similar function for essential bHLH proteins in lymphoid tissues, such as ABF-1 (Massari et al., Mol Cell Biol. 18(6), 3130-9 (1998)) and E2A (Greenbaum and Zhuang, Semin Immunol. 14:405-414 (2002)).

4) Acheron also regulates the expression of a number of proteases. For example, it induces a 8-10-fold increase in granzyme D and E.

These data suggest that Acheron could function in a number of disorders including but not limited to: cancer, inflammation, cell death, auto-immunity, and atherosclerotic disease, and that inhibition of Acheron expression or activity may be useful in treating these conditions.

### Example 17

### Optimizing the Blockade of Endogenous Acheron

Rationale: As described herein (see Example 3), blockade of the endogenous Acheron protein with a dominant-negative form (tAcheron) or antisense-Acheron enhances the formation of satellite cells and blocks apoptosis following trophic factor withdrawal. These properties make Acheron an ideal target for manipulations designed to enhance the utility of transplanted satellite cells.

To determine which method is optimal for inhibiting Acheron function, wild-type primary myoblasts are infected with one of four different experimental constructs: 1) constitutively expressed tAcheron; 2) transiently expressed tAcheron from the cyclic dependent protein kinase 2 (cdc2) or B-myb promoters; 3) antisense Acheron; and 4) small interfering Acheron RNA (siRNA). Each of these methods reduce endogenous Acheron and facilitate cell survival.

A) Constitutively expressed dominant negative tAcheron: A replication-defective pBabe-puromycin retrovirus is used to express ectopic tAcheron in myoblasts. These vectors use a MoMLV LTR (long terminal repeat) to drive high constitutive levels of expression. For this study, the pBabe-tAcheron construct described herein is used.

B) Antisense Acheron: As described herein, pBabe antisense-Acheron (AS-Acheron) protects myoblast cells from the loss of trophic support (Example 3, FIG. 2). However this construct was less effective than dominant-negative tAcheron. Consequently, the use of AS-Acheron may represent a good compromise between the conflicting needs of enhancing myoblast survival and the need to facilitate differentiation.

C) Transiently expressed tAcheron: As described herein, dominant-negative tAcheron blocks both death and differentiation of satellite cells. This raises the concern that while constitutive blockade of Acheron will enhance survival of ectopic cells following transplantation, it may inhibit optimal myogenesis. To address this problem, the MoMLV LTR from pBabe-tAcheron is replaced with the 5′ sequence from either the B-myb or cdc2 promoters, which are active during the G2/S phase of the cell cycle and then repressed in quiescent cells (Joaquin and Watson, J. Biol. Chem. 278(45):44255-64 (2003); Dalton, EMBO J. 11(5):1797-804 (1992); Liu et al., Circ. Res. 82(2):251-60 (1998). These manipulations should drive the expression of tAcheron while the cells are in growth medium, but not when the cells are exposed to low serum differentiation medium.

As a control, a cdc2 promoter-Green Fluorescent Protein (GFP) reporter construct is constructed and tested in the cell-based assays described herein. This will verify that transfer to low serum differentiation medium, which arrests cell division, results in a reduction in cdc2 promoter activity. To more accurately monitor promoter activity in real time, an engineered GFP protein that displays a very short half-life in cells due to enhanced ubiquitin/proteasome dependent degradation (Dantuma et al., Nat Biotechnol. 18(5):538-43 (2000)) is used.

D) siRNA: A 19 nucleotide sequence of the Acheron gene and its complementary sequence are subcloned into the pSUPER™ (OligoEngine Co.) mammalian expression vector. A short hairpin sequence separates the two self-complementary sequences. The RNA polymerase III HI promoter in the vector drives high levels of expression in mammalian cells where the short RNAi is cleaved and represses gene expression in a sequence-dependent manner (Brummelkamp et al., Cancer Cell. 2(3):243-7 (2002); Brummelkamp et al., Science. 296(5567):550-3 (2002)). In separate experiments, in vitro synthesized double stranded siRNAi against Acheron using the Silencer™ siRNA Construction Kit (Ambion) is used.

In Vitro Cell Assays: Each of the nucleic acid constructs described above in A-D is analyzed in the same primary mouse myoblasts employed for in vivo transplantation studies described in Example 16 below. C57Bl10J mice are sacrificed and primary myoblasts are prepared from the leg muscles of 2-3 day post-natal pups according to the methods of Rando and Blau (1994, supra; 1997, supra) to generate cultures that are greater than 98% pure myoblasts. Primary myoblasts are cultured in DMEM supplemented with 20% FBS, 0.5% chick embryo extract and antibiotics. Myoblast purity is determined by staining cultures with an antibody against desmin, a myoblast marker (Morris and Head, Exp Cell Res. 158(1):177-91 (1985)). After enzymatic dissociation of muscles with collagenase (0.2%) and trypsin (0.25%), the cells are cultured in high glucose DMEM at 37° C. for 3 days.

Cultures are expanded, split and transferred to new plates. Each plate is infected with one of the expression constructs described above. Two control viruses are also included: empty vector and pBabe expressing an irrelevant gene (GFP; Green Fluorescent Protein). The use of GFP has the added advantage of allowing one to assess infection efficiency. Retroviruses are packaged in Phoenix cells according to protocols from the Nolan laboratory (Yang et al. 1999) and introduced into the primary myoblasts according to the procedures described by Springer and Blau (Springer and Blau, Somat Cell Mol Genet. 23(3):203-9 (1997)) who reported greater than 99% infection efficiency. The efficiency of these methods has been confirmed.

After infecting the primary mouse myoblasts with each of these constructs, cells are plated in 96 well plates at 40% confluency and then allowed to reach 85% confluency before the growth medium (GM) is replaced with a 2% horse serum/DMEM differentiation medium (DM). Plates are assayed at various times after transfer, including: 0 hours, 12 hours, 24 hours, 48 hours, 72 hours and 96 hours. One set of plates is stained with calcein-AM and ethidium bromide heterodimer (“Live/Dead” Molecular Probes) and read on a fluorescence plate reader. The calcein-AM enters living cells and is de-esterified, which traps it in cells and induces fluorescence. The ethidium bromide heterodimer enters dead cells and fluoresces intensely when it intercalates into genomic DNA. Therefore live cells have green cytoplasm while dead cells have red nuclei. (Visual counts are also employed to insure that the readings from plate assays reflect the appearance of the cells). These experiments provide a quantitative measure of cell death in these cultures, to evaluate whether these genetic manipulations improve cell survival following removal of trophic support.

A second plate of engineered cells from each experiment is fixed and reacted with a monoclonal antibody to myosin heavy chain, a marker of myogenesis. Wells are incubated with a horse radish peroxidase labeled secondary antibody and the substrate 2,2-azino-di(3-ethylbenzthiazoline-6-sulfonic acid) (ABTS). Using the method of Shumway and Schwartz (Biotechniques.; 31(5):996, 998, 1000 (2001)), the levels of MHC expression are quantified on a microtiter plate reader as a measure of differentiation. After this stage, the ABTS are washed away and the cells reacted with the HRP substrate DAB. This allows visualization of the MHC-stained myotubes to assess the extent of myogenesis by counting the number of myotubes, the average number of included nuclei, etc. (Shumway and Schwartz, 2001, supra).

Results: All the manipulations that block endogenous Acheron expression reduce cell death relative to control cells following transfer to DM. Cultures expressing tAcheron from the cdc2 or B-Myb promoters will likely display the greatest levels of myotube formation in DM because the block to Acheron will be transient. These experiments indicate which manipulations are likely to be the ones with the greatest potential for enhancing the survival and differentiation of transplanted myoblasts.

### Example 18

### Effects of Blocking Acheron Function on Myoblast Survival and Proliferation In Vivo

Rationale: As described herein, blockade of Acheron function allows satellite cells to survive in the absence of trophic support. To develop this observation for potential therapeutic applications, these studies are extended into an in vivo animal model.

Methods: C57Bl10J primary mouse myoblasts are isolated from newborn males and prepared as described above. Males are specifically used so that when cells are transplanted into C57BL10J female hosts, the number of ectopic cells can be approximated by performing quantitative PCR with primers directed against Y chromosome-specific sequences using a technique previously described by the Tremblay laboratory (Caron et al., Biotechniques 27(3):424-6, 428 (1999)).

Primary myoblasts are expanded in vitro in GM and then infected with one of the pBabe retroviral vectors described in Aim I. Control myoblasts are either uninfected or infected with either an empty vector or a GFP expression vector. After retroviral infection, the myoblasts are expanded in vitro and incubated with 0.25 μCi/ml [methyl-¹⁴C] thymidine in growth medium 16-24 hours prior to transplantation. The radioactively labeled genetically engineered male myoblasts are then centrifuged for 5 minutes at 3500 rpm and resuspended in 15% horse serum, centrifuged for 10 minutes at 4000 rpm and resuspended in 10 μl of Hank's balanced salt solution (HBSS) in preparation for injection.

One million cells are injected in the Tibialis anterior (TA) muscle of female C57BL10J mice under deep anesthesia. Basically, an intravenous cannula is used to insert a 280 micron diameter polyethylene plastic tube into the muscle parallel to the fibers (El Fahime et al. 2000). The distal end of the tube is sealed and there are 4 small holes placed at 2 mm distances along the length of the tube. Cells are slowly injected from the proximal extremity of the polyethylene micro-tube with a glass micro-pipette (Drummond Scientific Co., Broomall, Pa.) with a 50 μm tip. The engineered cells are injected in a 10 μl volume which satisfies two criteria: first, this volume can be easily injected without causing tissue distortion or swelling; and second, it is 5 μl more than the volume of the micro-tube (5 μl), so that some cells will be expelled immediately from the tube. Non-engineered control myoblasts are injected into the contralateral TA muscles.

The muscles of 10 of these mice are removed immediately to establish the 100% value for the number of injected myoblasts. This controls for loss of label during cell transfer, as well as any quenching that may take place in the sample. Ten mice for each treatment group are sacrificed after 1, 3 and 5 days. The TA muscles are dissected out and a competitive PCR oligonucleotide is added to the muscle before DNA extraction. The ¹⁴C thymidine radioactivity is measured by scintillation counting as a measure of cell death (Beauchamp et al. 1999; Skuk et al. 2002). The presence of male cells in the muscle is quantified by real-time PCR. The competitive oligonucleotide is amplified with the same primers as the Y chromosome sequence and serves as a competitor to obtain a quantitative result (see Caron et al. 1999).

This set of experiments establishes the death of the injected myoblasts and the proliferation of the surviving cells at different time points in the same samples. The death of the myoblasts is established using the amount of radioactivity still present at different times as a percentage of the radioactivity present at time zero. This marker is divided among daughter cells during proliferation and cannot be used as an indicator of proliferation. Instead, the proliferation of the surviving transplanted male myoblasts is quantified by competitive PCR for the Y chromosome. Since host cells lack this sequence, the quantity of PCR product is proportional to the number of copies of the target DNA in the sample and should correlate well with the number of transplanted myoblasts that survive and proliferate. These results are evaluated by an analysis of variance to verify whether the engineering of the cells with various genes improves their in vivo survival and proliferation.

Results: Based on our in vitro studies, blockade of Acheron allows more cells to survive and proliferate in vivo. This is seen as both the retention of ¹⁴C in engineered myoblasts versus the controls and as an increase in the levels of Y-chromosome-specific DNA. These studies provide the first functional tests related to targeting Acheron for improving the survival of ectopic cells.

### Example 19

### Acheron Interacts with Ariadne and Parkin

COS-1 cells were co-transfected with cDNA constructs encoding: 1) c-myc-tagged Ariadne and FLAG-tagged Acheron; or 2) c-myc-tagged Parkin and FLAG-tagged Acheron. After 48 hours, cells were washed 2 times with phosphate buffered saline (PBS) and lysed at room temperature. Samples were clarified via centrifugation and anti c-myc monoclonal antibody (clone 9e10 monoclonal) added. Samples were incubated over night and then protein G Sepharose™ 4 fast flow beads were added. Samples were shaken at room temperature 1 hour, centrifuged, washed 2 times with PBS and then fractionated on a 4-15% Tris-HCL polyacrylamide gel. Proteins were transferred to Immobilon P and reacted with Western 1:1000 anti-Flag M5 monoclonal antibody. The inimunoprecipitation of Parkin or Ariadne precipitated Acheron as well.

These data support the hypothesis that Acheron binds to Parkin and Ariadne.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated engineered cell having an altered level of Acheron activity.

2. The isolated cell of claim 1, wherein the cell has reduced Acheron activity.

3. The isolated cell of claim 1, wherein the cell has increased Acheron activity.

4. The cell of claim 1, wherein the cell is a myoblast.

5. The cell of claim 1, wherein the cell is a neural stem cell.

6. The cell of claim 1, wherein the cell comprises an exogenous gene.

7. A method of preparing a cell for implantation into a recipient, the method comprising contacting the cell with an Acheron inhibitor in an amount effective to reduce Acheron expression or activity within the cell.

8. The method of claim 8, wherein the Acheron inhibitor is selected from the group consisting of an Acheron-specific antibody, an antisense nucleic acid complementary to an Acheron nucleic acid, a small inhibitory RNA that cleaves an Acheron mRNA, a ribozyme that cleaves an Acheron nucleic acid, a nucleic acid molecular that encodes a dominant negative Acheron polypeptide, and a dominant negative Acheron polypeptide.

9. A kit comprising an Acheron inhibitor selected from the group consisting of an Acheron-specific antibody, an antisense nucleic acid complementary to an Acheron nucleic acid, a small inhibitory RNA that cleaves an Acheron mRNA, a ribozyme that cleaves an Acheron nucleic acid, and a dominant negative Acheron polypeptide, and instructions for use in a method of preparing cells for transplantation.

10. A method of identifying a candidate compound for the treatment of a disorder associated with aberrant apoptosis or cellular differentiation, the method comprising:
providing an Acheron nucleic acid molecule or polypeptide;
contacting the Acheron nucleic acid molecule or polypeptide with a test compound under conditions in which the nucleic acids expression or polypeptide activity can be determined; and
evaluating any effect of the test compound on the expression of the Acheron nucleic acid or an activity of the Acheron polypeptide,
wherein a test compound that modulates the expression of the Acheron nucleic acid or an activity of the Acheron polypeptide is a candidate compound for the treatment of a disorder associated with apoptosis or cellular differentiation.

11. The method of claim 10, wherein the Acheron nucleic acid molecule or polypeptide is in a cell.

12. The method of claim 10, further comprising
selecting a candidate compound that increases expression of the Acheron nucleic acid or the activity of the Acheron polypeptide; and
evaluating the candidate compound in a mammal having a disorder associated with aberrant cellular proliferation.

13. The method of claim 12, wherein the mammal is a human subject in a clinical trial.

14. The method of claim 10, further comprising
selecting a compound that decreases the expression of the Acheron nucleic acid or the activity of the Acheron polypeptide; and
evaluating the compound in a mammal having a disorder associated with aberrant cellular degeneration.

15. The method of claim 14, wherein the mammal is a human subject in a clinical trial.

16. The method of claim 14, wherein the disorder is muscular dystrophy.

17. An isolated nucleic acid molecule selected from the group consisting of
a) an isolated nucleic acid molecule that encodes an Acheron polypeptide comprising a sequence of 5 to 490 contiguous amino acids within SEQ ID NO:4, wherein the polypeptide has a measurable affect on apoptosis or cellular differentiation that is at least 25% of the measured affect of the full-length Acheron polypeptide, and
b) an isolated nucleic acid molecule that encodes a dominant negative Acheron polypeptide comprising a sequence of 5 to 457 contiguous amino acids within amino acid locations 34-491 of SEQ ID NO:4.

18. A vector comprising the nucleic acid molecule of claim 17.

19. The vector of claim 18, further comprising a nucleic acid sequence encoding a heterologous polypeptide.

20. A host cell that contains the nucleic acid molecule of claim 17.

21. An isolated polypeptide selected from the group consisting of:
a) an Acheron polypeptide comprising a sequence of 5 to 490 contiguous amino acids within SEQ ID NO:4, wherein the polypeptide has a measurable affect on apoptosis or cellular differentiation that is at least 25% of the measured affect of the full-length Acheron polypeptide; and
b) a dominant negative Acheron polypeptide comprising a sequence of 5 to 457 contiguous amino acids within amino acid locations 34-491 of SEQ ID NO:4.

22. The polypeptide of claim 21, further comprising a heterologous amino acid sequence.

23. An isolated antibody or antigen-binding portion thereof that binds to an Acheron polypeptide.

24. The isolated antibody of claim 23, wherein the antibody is a monoclonal, polyclonal, or monospecific antibody.

25. The isolated antigen-binding portion of claim 23, wherein the antigen-binding portion is an Fv, Fab, or F(ab)₂.

26. A method of treating a subject in need of a cellular implant, the method comprising administering to the subject an effective amount of cells having reduced Acheron activity.

27. A method of treating a subject having a disorder associated with abnormal cellular degeneration, the method comprising administering to the subject cells comprising an amount of an Acheron inhibitor effective to reduce Acheron activity in the cells compared to wild type cells.

28. The method of claim 27, wherein the Acheron inhibitor comprises wherein the Acheron inhibitor is selected from the group consisting of an Acheron-specific antibody, an antisense nucleic acid complementary to an Acheron nucleic acid, a small inhibitory RNA that cleaves an Acheron mRNA, a ribozyme that cleaves an Acheron nucleic acid, a nucleic acid molecular that encodes a dominant negative Acheron polypeptide, and a dominant negative Acheron polypeptide.


SEQUENCE LISTING (23 sequences)

SEQ ID NO: 1 - DNA, 2247 bp, Manduca sexta
ggcacgaggcccgacgaaggcatcgccaccgaccgccgcccgtccaccgacgaacatgccgacctctccgataccgcctccgaggcgggctccggcggcggcaaagactcgggatgtgaggtggcgcccgataccaacgaaccgccctacacgccgcccgacgacgagctcgccaacaggatcgtctcgcaggtcgagttctacttctccgacgccaacattactaaggatgctttcctgctcaaacacgtccgccgcaacaaggaggggtacgtctccctcaaattgatatcgagcttcaaacgagtcaagcatctcaccaaggactggagggttgtagcagaggcgctcaagagatccaccaagctggagataaacgaactgggaacgaagcttcgcaggatagatccgctcccggcttacgacgagacgacaccgtccaggaccgtggtggcggtgcggatgccgatcgaaaggccgtcggtggagaatgtgtcaaggctgttcgcgggctgcggcgagatcgccctggtgcgagtgttgcggcccggcaacccggtgcccgcggatgtgcgccaattcctcaacaagaacccgagtttggttaactgtgtgtgtgccttagttgagtttacggaatcggaggccgcgcgggaggcgctcaggctgcaaagcccggacgaggaaggtatgcgggtgtacgaactgaacggagtaccgcgcgagcccaagaggaaggcgccggtccgccggacgcctcagcggcgtcacgagtgtgaatactcctcatgttgcagcggctccgagcctgaatacgatttcagatatggaacaccattctacagaagaaattccagcgggttcttcgcaccgcgatcgccagaaatacagacatgggtaccgcggcgccagtccacctgcagccacagctcggactcgggcgtgtcgttctactgcaactcgaggagagcgtcgcaagctagcacgggcagcgccagcagcgcggagggctggctggcgcggcggctgtccgggtgctcgctttcgggaacggagtgcggtggacggcggctgtcgtgcgcgccgcggttcgagccgcgcacgccgctggtgcccgacggtacgaggggcttccacgccgccgcgcgccagcggagaatctcggacctcgcgttgtattcgcgctagaaagcgcccgccaaccgtgacacctttctaatcacgttggataactgcctttcacgttctgcccgcgtagttttttaatctatttggtacgaggaaacgacgtggtatagtttcgatcgcgtgtgtgtcgttggacagtgcgaatgtttagttttcctcgtgacagaagtgaagcgcggttaccgaccgtcgatgccgagttaccttcacctctccgtttttgtacatgttgacgtatgtgattgttaagattagtgtaggttgttaactgtaagtttagcattaagccgtgagacgacaggtcgctgtgttagaagttaatttataatcatgtttaaatgtaatttattagattaatttattgtgtacttcgcgtatttattgcgtgaatgcgcgcgcgcgtcgcggtcgagttttcatccgagttatgtcagagttgttagcacaaaaccgacatacaaagtcgacattgagcgcgattgtagactctagaagcgtctggcgcgcctggcgtctgtggcgacgtctcgcgccagatgatcgcgtcgataacttacggcggagcagcagcgcggcatgcggcatgacgctgtggcggtgttcgagcacactgcacacgttgcgtaacttgttgcactgttcacgtgtgcacggagcaccagccgacagatttcctgcaaaagtaaaaggccgctctgcgactctgcctcgactgtgatagccgcggcgcgttggcggtactaacagattttacattttttatacgtctaaactcattaagtcattaatataatttaagtcagtcgcgccatttcatgtaattcattcatgttaagtgttgttcggtagggtactgtctgattgtgggccggccgcgtccggtttgcgtaactatactttcgtcgcactcgtgcgatacatatttttatttatttcctttaagagacgattgttacgccattttatgctttgcaacggcacaattaatttgcttatgtttttgtatgaagcgacaacaactgtcgttgatgtgattattttaatataattataagtttt

SEQ ID NO: 2 - protein, 395 aa, Manduca sexta
GTRPDEGIATDRRPSTDEHADLSDTASEAGSGGGKDSGCEVAPDTNEPPYTPPDDELANRIVSQVEFYFSDANITKDAFLLKHVRRNKEGYVSLKLISSFKRVKHLTKDWRVVAEALKRSTKLEINELGTKLRRIDPLPAYDETTPSRTVVAVRMPIERPSVENVSRLFAGCGEIALVRVLRPGNPVPADVRQFLNKNPSLVNCVCALVEFTESEAAREALRLQSPDEEGMRVYELNGVPREPKRKAPVRRTPQRRHECEYSSCCSGSEPEYDFRYGTPFYRRNSSGFFAPRSPEIQTWVPRRQSTCSHSSDSGVSFYCNSRRASQASTGSASSAEGWLARRLSGCSLSGTECGGRRLSCAPRFEPRTPLVPDGTRGFHAAARQRRISDLALYSR

SEQ ID NO: 3 - DNA, 2057 bp, Homo sapiens
  CDS (71)..(1546)
cagtcactgccgaccggctggctgggccttgcggcgtgaggaccccggcggcgccgcagtcccgcgagccatggcccagtccggcggggaggctcggcccgggcccaagacggcggtgcagatccgcgtcgccatccaggaggccgaggacgtggacgagttggaggacgaggaggagggggcggagactcggggcgccggggacccggcccggtacctcagccccggctggggcagcgcgagcgaggaggagccgagccgcgggcacagtggcaccactgcaagtggaggtgagaacgagcgtgaggacctggagcaggagtggaagcccccggatgaggagttgatcaagaaactggtggatcagatcgaattctacttttctgatgaaaacctggagaaggacgcctttttgctaaaacacgtgaggaggaacaagctgggatatgtgagcgttaagctactcacatccttcaaaaaggtgaaacatcttacacgggactggagaaccacagcacatgctttgaagtattcagtggtccttgagttgaatgaggaccaccggaaggtgaggaggaccacccccgtcccactgttccccaacgagaacctccccagcaagatgctcctggtctatgatctctacttgtctcctaagctgtgggctctggccaccccccagaagaatggaagggtgcaagagaaggtgatggaacacctgctcaagcttttcgggacttttggagtcatctcatcagtgcggatcctcaaacctgggagagagctgccccctgacatccggaggatcagcagccgctacagccaagtggggacccaggagtgtgccatcgtggagttcgaggaggtggaagcagccatcaaagcccatgagttcatgatcacagaatctcagggcaaagagaacatgaaagctgtcctgattggtatgaagccacccaaaaagaaacctgccaaagacaaaaatcatgacgaggagcccactgcgagcatccacctgaacaagtccctgaacaagagagtcgaggagcttcagtacatgggtgatgagtcttctgccaacagctcctctgaccccgagagcaaccccacatcccctatggcgggccgacggcacgcggccaccaacaagctcagcccgtctggccaccagaatctctttctgagtccaaatgcctccccgtgcacaagtccttggagcagccccttggcccaacgcaaaggcgtttccagaaagtccccactggcggaggaaggtagactgaactgcagcaccagccctgagatcttccgcaagtgtatggattattcctctgacagcagcgtcactccctctggcagcccctgggtccggaggcgtcgccaagccgagatggggacccaggagaaaagccccggtacgagtcccctcctctcccggaagatgcagactgcagatgggctacccgtaggggtgctgaggttgcccaggggtcctgacaacaccagaggatttcatggccatgagaggagcagggcctgtgtataaataccttctatttttaatacaagctccactgaaaaccaccttcgttttcaaggttctgacaaacacctggcatgacagaatggaattcgttcccctttgagagattttttattcatgtagacctcttaatttatctatctgtaatatacataaatcggtacgccatggtttgaagaccaccttctagttcaggactcctgttcttcccagcatggccactattttgatgatggctgatgtgtgtgagtgtgatggccctgaagggctgtaggacggaggttccctgggggaagtctgttctttggtatggaatttttctctcttctttggtatggaatttttcccttcagtgactgagctgtcctcgataggccatgcaagggcttcctgagagttcaggaaagttctcttgtgcaacagcaagtagctaagcctatagcatggtgtcttgtaggaccaaatcgatgttacctgtcaagtaaataaataataaaacaccaaaaaaaaaaaa

SEQ ID NO: 4 - protein, 491 aa, Homo sapiens
MAQSGGEARPGPKTAVQIRVAIQEAEDVDELEDEEEGAETRGAGDPARYLSPGWGSASEEEPSRGHSGTTASGGENEREDLEQEWKPPDEELIKKLVDQIEFYFSDENLEKDAFLLKHVRRNKLGYVSVKLLTSFKKVKHLTRDWRTTAHALKYSVVLELNEDHRKVRRTTPVPLFPNENLPSKMLLVYDLYLSPKLWALATPQKNGRVQEKVMEHLLKLFGTFGVISSVRILKPGRELPPDIRRISSRYSQVGTQECAIVEFEEVEAAIKAHEFMITESQGKENMKAVLIGMKPPKKKPAKDKNHDEEPTASIHLNKSLNKRVEELQYMGDESSANSSSDPESNPTSPMAGRRHAATNKLSPSGHQNLFLSPNASPCTSPWSSPLAQRKGVSRKSPLAEEGRLNCSTSPEIFRKCMDYSSDSSVTPSGSPWVRRRRQAEMGTQEKSPGTSPLLSRKMQTADGLPVGVLRLPRGPDNTRGFHGHERSRACV

SEQ ID NO: 5 - protein, 982 aa, Homo sapiens
MAQSGGEARPGPKTAVQIRVAIQEAEDVDELEDEEEGAETRGAGDPARYLSPGWGSASEEEPSRGHSGTTASGGENEREDLEQEWKPPDEELIKKLVDQIEFYFSDENLEKDAFLLKHVRRNKLGYVSVKLLTSFKKVKHLTRDWRTTAHALKYSVVLELNEDHRKVRRTTPVPLFPNENLPSKMLLVYDLYLSPKLWALATPQKNGRVQEKVMEHLLKLFGTFGVISSVRILKPGRELPPDIRRISSRYSQVGTQECAIVEFEEVEAAIKAHEFMITESQGKENMKAVLIGMKPPKKKPAKDKNHDEEPTASIHLNKSLNKRVEELQYMGDESSANSSSDPESNPTSPMAGRRHAATNKLSPSGHQNLFLSPNASPCTSPWSSPLAQRKGVSRKSPLAEEGRLNCSTSPEIFRKCMDYSSDSSVTPSGSPWVRRRRQAEMGTQEKSPGTSPLLSRKMQTADGLPVGVLRLPRGPDNTRGFHGHERSRACVMAQSGGEARPGPKTAVQIRVAIQEAEDVDELEDEEEGAETRGAGDPARYLSPGWGSASEEEPSRGHSGTTASGGENEREDLEQEWKPPDEELIKKLVDQIEFYFSDENLEKDAFLLKHVRRNKLGYVSVKLLTSFKKVKHLTRDWRTTAHALKYSVVLELNEDHRKVRRTTPVPLFPNENLPSKMLLVYDLYLSPKLWALATPQKNGRVQEKVMEHLLKLFGTFGVISSVRILKPGRELPPDIRRISSRYSQVGTQECAIVEFEEVEAAIKAHEFMITESQGKENMKAVLIGMKPPKKKPAKDKNHDEEPTASIHLNKSLNKRVEELQYMGDESSANSSSDPESNPTSPMAGRRHAATNKLSPSGHQNLFLSPNASPCTSPWSSPLAQRKGVSRKSPLAEEGRLNCSTSPEIFRKCMDYSSDSSVTPSGSPWVRRRRQAEMGTQEKSPGTSPLLSRKMQTADGLPVGVLRLPRGPDNTRGFHGHERSRACV

SEQ ID NO: 6 - DNA, 1476 bp, Homo sapiens
atggcccagtccggcggggaggctcggcccgggcccaagacggcggtgcagatccgcgtcgccatccaggaggccgaggacgtggacgagttggaggacgaggaggagggggcggagactcggggcgccggggacccggcccggtacctcagccccggctggggcagcgcgagcgaggaggagccgagccgcgggcacagtggcaccactgcaagtggaggtgagaacgagcgtgaggacctggagcaggagtggaagcccccggatgaggagttgatcaagaaactggtggatcagatcgaattctacttttctgatgaaaacctggagaaggacgcctttttgctaaaacacgtgaggaggaacaagctgggatatgtgagcgttaagctactcacatccttcaaaaaggtgaaacatcttacacgggactggagaaccacagcacatgctttgaagtattcagtggtccttgagttgaatgaggaccaccggaaggtgaggaggaccacccccgtcccactgttccccaacgagaacctccccagcaagatgctcctggtctatgatctctacttgtctcctaagctgtgggctctggccaccccccagaagaatggaagggtgcaagagaaggtgatggaacacctgctcaagcttttcgggacttttggagtcatctcatcagtgcggatcctcaaacctgggagagagctgccccctgacatccggaggatcagcagccgctacagccaagtggggacccaggagtgtgccatcgtggagttcgaggaggtggaagcagccatcaaagcccatgagttcatgatcacagaatctcagggcaaagagaacatgaaagctgtcctgattggtatgaagccacccaaaaagaaacctgccaaagacaaaaatcatgacgaggagcccactgcgagcatccacctgaacaagtccctgaacaagagagtcgaggagcttcagtacatgggtgatgagtcttctgccaacagctcctctgaccccgagagcaaccccacatcccctatggcgggccgacggcacgcggccaccaacaagctcagcccgtctggccaccagaatctctttctgagtccaaatgcctccccgtgcacaagtccttggagcagccccttggcccaacgcaaaggcgtttccagaaagtccccactggcggaggaaggtagactgaactgcagcaccagccctgagatcttccgcaagtgtatggattattcctctgacagcagcgtcactccctctggcagcccctgggtccggaggcgtcgccaagccgagatggggacccaggagaaaagccccggtacgagtcccctcctctcccggaagatgcagactgcagatgggctacccgtaggggtgctgaggttgcccaggggtcctgacaacaccagaggatttcatggccatgagaggagcagggcctgtgtataa

SEQ ID NO: 7 - DNA, 2042 bp, Mus musculus
gctctgccgcgcgcgcgccgcccacgcgcgatcgtcgctatcgagggctgccggctggcctggcctcgcgacacggagaccctgccaaccatggcccagctcggcgaacagactctgcctgggcccgagaccacggtgcagatccgtgtcgccatccaggaggctgaggatctggaggatctggaggaggaggacgaggggacctcggcgcgggcagcgggggacccagcccggtacctcagtcccggctggggcagcgccagcgaggaggagccgagccgcgggcacagtagtgccacgacaagtgggggcgagaacgatcgcgaggacctggagcctgagtggaggcccccggacgaggagctcatcaggaagctggtggatcagattgagttctacttttcggacgagaacctggagaaggacgccttcctgctgaagcacgtgcggaggaacaagctgggctacgtgagcgtcaagctgctcacctccttcaagaaggtgaaacacctcacccgggactggaggaccacagcacacgccttgaagtattcagtcaccctggagttgaacgaggaccaccggaaggttaggaggaccacccctgtgccactgttccccaatgagaacctccccagcaagatgctgctggtctatgacctacacctgtcccctaagctctgggccctggccacaccccagaagaacggaagggtgcaggagaaggtgatggagcatctgctcaagctctttgggacttttggcgtcatctcatcggtgcggatcctaaaacctgggagagagctgccccctgacatccggaggatcagcagccgctacagccaggtggggacccaagagtgcgccattgtggagttcgaggaggtggacgcggccattaaagcccatgaattcatggtcactgaatctcagagcaaagagaacatgaaggctgttctgattgggatgaagccgcccaaaaagaaacccctcaaagataagaaccatgacgatgaggccacagcaggtacccacctaagcagatccctgaacaagagagtggaggaacttcagtacatgggggatgagtcttccgccaacagctcctctgaccctgagagcaatcccacctctcccatggccggccggcggcacgcggccagcaacaagctcagcccttcgggccaccagaatatttttctgagccccaatgcctccccgtgctcaagcccatggagcagccccttggcacagcgcaagggtgtctccagaaaatccccgctggctgaagaaggtagactgaacttcagcaccagccctgagatcttccgaaagtgcatggattattcttccgacagcagcatcactccctcgggcagcccctgggttcgcagacgacgccaggctgagatggggactcaggagaaaagtccaggggcgagtcccctgctgtctcggaggatgcagaccgcagatgggttacctgtgggggtgctgagactgcccagaggccccgacaacaccaggggcttccacggtggacatgagagaggcagagcctgtgtataatgccttctattttttaataccagctccatcggaaaccgtctttgttttcgagatcctcactaatagctagcatgacagagaatggagttcagtccccttagaaagcttttgtatccatgtagacctcttaatttatatatttgtaaggtatacaaactgtctggtgggccatgggtttaggatcgtcttctggctggggctgttgctctcagcaaggccactgttctgtcaatgcttggcatgtgttagtgtggtggctctgaagggctgtgggacagaggatctctggaaagatctagtagtgtcggaccgtttttttcttacaatgactgagctgtctttggcaggccgcgcaagggctcctcttaagacctcaagggagatgtgctttatggtaaatcctacagtcaatagcatggtgtctcataggactgagtgtgtctgttccctgtcaagtgaataaataataaaac

SEQ ID NO: 8 - protein, 492 aa, Mus musculus
MAQLGEQTLPGPETTVQIRVAIQEAEDLEDLEEEDEGTSARAAGDPARYLSPGWGSASEEEPSRGHSSATTSGGENDREDLEPEWRPPDEELIRKLVDQIEFYFSDENLEKDAFLLKHVRRNKLGYVSVKLLTSFKKVKHLTRDWRTTAHALKYSVTLELNEDHRKVRRTTPVPLFPNENLPSKMLLVYDLHLSPKLWALATPQKNGRVQEKVMEHLLKLFGTFGVISSVRILKPGRELPPDIRRISSRYSQVGTQECAIVEFEEVDAAIKAHEFMVTESQSKENMKAVLIGMKPPKKKPLKDKNHDDEATAGTHLSRSLNKRVEELQYMGDESSANSSSDPESNPTSPMAGRRHAASNKLSPSGHQNIFLSPNASPCSSPWSSPLAQRKGVSRKSPLAEEGRLNFSTSPEIFRKCMDYSSDSSITPSGSPWVRRRRQAEMGTQEKSPGASPLLSRRMQTADGLPVGVLRLPRGPDNTRGFHGGHERGRACV

SEQ ID NO: 9 - protein, 8 aa, Artificial
  FLAG synthetic peptide
DYKDDDDK

SEQ ID NO: 10 - DNA, 2694 bp, mus musculus
atggccgacgacgacgtgctgttcgaggatgtgtacgagctatgcgaggtgatcggcaagggtcccttcagtgttgtacggcgatgtatcaacagagaaactgggcaacaatttgctgtaaaaattgttgatgtagccaagttcacatcaagtccagggttaagtacagaagatctaaagcgggaagccagtatctgtcatatgctgaagcatccacacattgtagagctgttggagacatatagctcagatgggatgctttacatggtttttgaatttatggatggagcagatctgtgttttgaaatcgtaaagcgagctgatgctggttttgtatacagtgaagctgtagccagccactacatgagacagatactggaagctctgcgctactgtcatgataataacataattcacagggacgtgaagccccactgtgttctccttgcctcaaaagaaaactcggcacctgttaaacttgggggctttggggtggccattcagttaggagaatctggacttgttgctggaggccgcgttggaacacctcactttatggccccagaagtggtcaagagagagccttacggaaagcctgtggatgtctggggctgtggtgtgatccttttcatcctgctcagtggctgtttgcctttctacggaaccaaggaaagattgtttgaaggcattattaaaggaaaatataagatgaatccaaggcagtggagccatatctctgaaagtgccaaagacctagtacgccgcatgctgatgctggatcctgctgaaaggatcactgtttatgaagcactgaatcacccatggcttaaggagcgggatcgttatgcctacaaaatccatcttccagaaacagtagaacaactgaggaaattcaatgcaaggagaaaactaaagggtgcagtactagctgctgtgtcaagtcacaaattcaattccttctatggggaccctcctgaagagttgccagatttctccgaagaccctacctcctcaggagccgtctctcaggtgctggacagcctggaagagattcacgcacttacagactgcagtgaaaaggacctagattttctacacagtgttttccaggatcaacatcttcacacactgctggatctgtatgacaaaattaacacaaagtcttcgccacaaatcaggaatcctccaagtgatgcagtacagagagccaaagaggtattggaagaaatttcatgttaccctgagaataatgatgcgaaggaactaaagcgtattttaacacaacctcatttcatggccttacttcagactcatgatgtagtggcacatgaagtttacagtgatgaagcattaagggtcacacctcccccgacttccccctatttaaacggtgattctccagaaagtgcaaacggagacatggacatggagaatgtgaccagagttcggctggtacagttccaaaagaacacggatgagccaatgggaatcactttgaaaatgaatgagctaaatcattgtattgtcgcaagaatcatgcatgggggtatgattcacaggcaaggtacacttcatgttggtgatgaaatccgagaaatcaatggcatcagtgtcgctaaccaaacagtggaacagctacagaaaatgcttagggaaatgcgagggagtattaccttcaagattgtgccaagctaccgcactcagtcttcgtcctgtgaggacttgccatcaaccacccaaccaaaaggacgacagatctatgtaagagcacaatttgaatatgatccagccaaggatgacctcatcccctgcaaagaagctggcatccggttccgagttggtgacatcatccagattattagtaaggatgaccacaactggtggcagggtaaactggaaaactccaaaaatggaactgcaggtctcattccttctcctgaacttcaggaatggcgagtagcttgcattgccatggagaagaccaaacaagagcagcaggccagctgtacttggtttggcaagaaaaagaagcagtacaaagataaatatttggcaaagcacaacgcagtgtttgatcaattagatcttgtcacatatgaagaagtagtcaaactgccagcattcaaaaggaaaacattagtcttattaggtgcacatggtgttggaagaagacacataaaaaataccctcatcacaaagcacccggaccggtttgcgtaccctattccacatacaaccagacctccaaagaaagatgaagaaaatggcaagaattattactttgtatctcatgaccaaatgatgcaagacatctcaaataacgaatactcggagtatggcagccatgaggatgcaatgtacgggacaaaactggagaccattcggaaaatccatgagcaggggctgattgcgattctggacgtggagcctcaggcactgaaggtcctgaggactgcagagtttgctccttttgttgtcttcattgcggcgccaactatcactccaggtttaaatgaggatgaatctcttcagcgcctgcagaaggagtccgatgtcttgcagagaacatatgcacactacttcgatctcacaattatcaacaacgaaattgatgagacaatcagacatctggaagaagctgtcgagcttgtgtgcacagccccacagtgggtcccagtctcctgggtctattag

SEQ ID NO: 11 - protein, 897 aa, Mus musculus
MADDDVLFEDVYELCEVIGKGPFSVVRRCINRETGQQFAVKIVDVAKFTSSPGLSTEDLKREASICHMLKHPHIVELLETYSSDGMLYMVFEFMDGADLCFEIVKRADAGFVYSEAVASHYMRQILEALRYCHDNNIIHRDVKPHCVLLASKENSAPVKLGGFGVAIQLGESGLVAGGRVGTPHFMAPEVVKREPYGKPVDVWGCGVILFILLSGCLPFYGTKERLFEGIIKGKYKMNPRQWSHISESAKDLVRRMLMLDPAERITVYEALNHPWLKERDRYAYKIHLPETVEQLRKFNARRKLKGAVLAAVSSHKFNSFYGDPPEELPDFSEDPTSSGAVSQVLDSLEEIHALTDCSEKDLDFLHSVFQDQHLHTLLDLYDKINTKSSPQIRNPPSDAVQRAKEVLEEISCYPENNDAKELKRILTQPHFMALLQTHDVVAHEVYSDEALRVTPPPTSPYLNGDSPESANGDMDMENVTRVRLVQFQKNTDEPMGITLKMNELNHCIVARIMHGGMIHRQGTLHVGDEIREINGISVANQTVEQLQKMLREMRGSITFKIVPSYRTQSSSCEDLPSTTQPKGRQIYVRAQFEYDPAKDDLIPCKEAGIRFRVGDIIQIISKDDHNWWQGKLENSKNGTAGLIPSPELQEWRVACIAMEKTKQEQQASCTWFGKKKKQYKDKYLAKHNAVFDQLDLVTYEEVVKLPAFKRKTLVLLGAHGVGRRHIKNTLITKHPDRFAYPIPHTTRPPKKDEENGKNYYFVSHDQMMQDISNNEYSEYGSHEDAMYGTKLETIRKIHEQGLIAILDVEPQALKVLRTAEFAPFVVFIAAPTITPGLNEDESLQRLQKESDVLQRTYAHYFDLTIINNEIDETIRHLEEAVELVCTAPQWVPVSWVY

SEQ ID NO: 12 - protein, 13 aa, Artificial
  Acheron Motif I
KDAFLLKHVRRNK

SEQ ID NO: 13 - protein, 7 aa, Artificial
  Acheron Motif II
XRXLXPG

SEQ ID NO: 14 - protein, 6 aa, Artificial
  Acheron Motif III
CAXVEX

SEQ ID NO: 15 - protein, 107 aa, homo sapiens
RTVVAVRMPIERPSVENVSRLFAGCGEIALVRVLRPGNPVPADVRQFLNKNPSLVNCVCALVEFTESEAAREALRLQSPDEEGMRVYELNGVPREPKRKAPVRRTPQ

SEQ ID NO: 16 - protein, 4 aa, Homo Sapiens
AGRR

SEQ ID NO: 17 - protein, 7 aa, Homo Sapiens
PKKKPAK

SEQ ID NO: 18 - protein, 8 aa, Homo Sapiens
LLVYDLYL

SEQ ID NO: 19 - DNA, 21 bp, artificial
  PCR primer
gtgcccgcggctcggctcctc

SEQ ID NO: 20 - DNA, 20 bp, artificial
  PCR primer
tccccggcgccccgagtctc

SEQ ID NO: 21 - DNA, 21 bp, artificial
  PCR primer
cggtacctcagccccggctgg

SEQ ID NO: 22 - protein, 867 aa, Drosophila melanogaster
MSSPRDKAEQVYEVKAPHAGAEGHTFLVSPVDPTPILAPILTAVPVMKAPDSPASMDKTKEMEELDDEQPSCSSQAVVPLSSSLPCKLLRMHARRSAQALLHQMESMDSQLGSSNGSTSEDTSPSAPPMSPTDHQLVDVGMGSSIGDSDESSEEGDELKPLAVDNHIIAEVDLTERQTTPPAVLSEANLEKFRKHQMDNIYLHPNFTLDASPPPAVAPANSPVLEAKRTHRSFLTMKKEKEVEPPQEKEQEVPVHQEQDTEPQGPNEIDTLDPSLIPSEELAAEITDAVEFYFSNESILKDAFLLKHVRRNKEGFVSLKLVSSFKRVRQLTREWKVVGDAVRRKSRKIELNDVGTKVRRIEPLPSFDETMPSRTIVACDLPLDKLTIEKVSDLFSPCGEIALIRILKPGMAIPVDVRQFMNKYPELQQKECALVEYLESSSARDARHLNGPFQVYEMVAPKKKTGKKAAVIQIAAPVARMVENYRYYNDANYERSRGGSFSGHETVPDLRFKLKRNNSDFQPSYYQQTGPSYHANPYQHYQPRGSIGNQNQEVGPNGFFGYGPRRYSNTSTISANTAAALGDVSPISSAVNSGGNPMVSGISSLQRRLSNCSEQNYTPEVNPSMSRRASNCSETGGVPQRRDSNCSESCPCSRRVSDFAQTTDTSYRKTSVCSNGSCPGNNQNQERRFSNGSMQFERTFSNASESSGFYRRPSNDFNIEREPIQTDQLVGGGGGGYQVWPRRYSNNFQQLSSKLAAYDNAQYIGGRRISTDSGYDRRCSFGSEGFEGSPRSRTGSFLSNYKHGGGDGYDGQPRSRTGSFLDGSPRSRSGSFAQRAAESLVRTPMGPDGSKGFGQRARKFGQTISPVN

SEQ ID NO: 23 - DNA, 2604 bp, Drosophila melanogaster
atgagcagtcccagggataaggcggagcaggtgtacgaggtgaaggctccacatgccggtgcagagggtcacacgttccttgtgagtcccgtggaccccactccgatcctagcgcccattctcaccgctgtgcctgtgatgaaagcaccggattcgcctgcttcaatggacaagaccaaggagatggaggagctggatgatgagcagccgagctgcagttcccaggcggtggtgccgttgtcctcatcgctgccctgcaagctgctccgcatgcatgccagacgctcggcccaggcgctgctccaccaaatggaatcgatggactctcagctgggcagcagcaatggcagcacctcggaggatacttcaccctctgctccgcccatgtcgcccactgaccaccaactggtggatgtgggcatgggttcctccattggggactcggatgaatcgtcggaagagggtgatgagctgaagcccctggccgtggacaatcacatcatagccgaggtggacctaaccgagcgccagactacgccaccggccgtcttgagtgaggcgaatttggagaagttccgtaagcaccagatggataacatctatctacatcctaacttcactctggatgcttctccacctccggcggtcgcaccagccaactcacccgttctggaggccaagagaacccatcgttcgttcctcaccatgaagaaggaaaaggaggtggagccgccgcaagagaaggagcaggaagtgccggtacaccaggaacaggatactgagccccaaggacccaatgagatagacacactagatccctcactcattcccagcgaggaactggcagccgaaatcaccgatgccgtggagttctacttctccaatgagagcatcctgaaggatgcattcctgctgaaacatgtgcgacgcaacaaggagggtttcgtcagccttaaactggtgtccagcttcaagagggtgcgccaactgaccagggagtggaaggtggttggagatgcagtgcgtcgcaagtcccgcaagatcgagctgaatgatgtgggcaccaaggtgcgcagaatcgaaccgctgcccagtttcgatgaaaccatgccctctcgcacgatcgttgcctgtgatctgccactggacaagctaaccatcgaaaaggtatcggatctattctcgccatgcggagagattgccctcatacgcattctcaagcccggaatggccattcccgtggatgtgcgtcagttcatgaacaagtatccagaactgcagcaaaaggagtgtgctctagtggagtacttagaatcctcttccgccagggatgcccgtcatctgaatggcccctttcaggtctacgagatggtggcgcccaagaaaaagaccggcaagaaggcagccgtcatccagatcgccgctccggtggcccggatggtggagaattatcgctactacaacgatgccaactacgagcggagtcgaggtggcagcttctccggccacgaaactgtaccggatctgcgcttcaaactcaagcgcaacaactcggacttccagcccagttactaccaacaaacggggcccagctaccatgccaatccgtatcagcattatcagccccgtgggagcattggcaaccagaaccaggaggtgggtcccaatggttttttcggctatggtcccaggcgatacagcaacacatcgacaatttcggccaacacagcggccgccttgggcgatgtttcgcctatatcttctgctgtcaattcaggcggtaatccgatggtgtctggtataagcagtctgcagcgccgtctgtccaactgctccgagcagaactacactccagaagtcaatccttcgatgtcacgaagggccagcaattgctcggaaacgggaggagttccccaacgtcgcgattcgaactgttcggagagttgtccatgctcccgaagggtctcggactttgctcagacgacagacactagttacagaaagacctcggtgtgttcgaatggcagctgtccgggcaataatcagaaccaggagcgtcgcttctccaacggatccatgcagttcgagcgaaccttctcgaatgccagcgagagcagtggcttctatcgtcgcccgtcgaacgatttcaatatcgagcgggagcccatccaaaccgatcagctggtgggtggaggaggcggtggctaccaggtgtggccacgtcgttactccaacaacttccagcagctgagcagcaagctggcggcatatgataatgctcagtatatcggaggaagacgtatatccacggactcgggatacgatcgtcgctgctcgtttggatcggaaggcttcgaaggatctccacgatctcgcaccggtagcttcctgagcaactacaagcacggcggcggcgatggctacgatggccagccaagatcccgaaccggtagcttcctcgacggatctcctcggtcgcgttccggttcgtttgcccagcgagctgccgagagcctggtgcgcactccaatgggtccagatggtagcaagggattcggccagagggccaggaaattcggacagaccatatcgcccgtaaattag